SEQUENCE LISTING (41 sequences)

SEQ ID NO: 1 - protein, 11 aa, Artificial Sequence
  Description of Artificial Sequence Synthetic peptide
GGGGDPGGGGR

SEQ ID NO: 2 - protein, 11 aa, Artificial Sequence
  Description of Artificial Sequence Synthetic peptide
AGSLDPAGSLR

SEQ ID NO: 3 - protein, 13 aa, Artificial Sequence
  Description of Artificial Sequence Synthetic peptide
AGSMLDPAGSMLR

SEQ ID NO: 4 - protein, 11 aa, Artificial Sequence
  Description of Artificial Sequence Synthetic peptide
AGSLADPGSLR

SEQ ID NO: 5 - protein, 11 aa, Artificial Sequence
  Description of Artificial Sequence Synthetic peptide
ALSLADPGSGR

SEQ ID NO: 6 - protein, 11 aa, Artificial Sequence
  Description of Artificial Sequence Synthetic peptide
ALSLGDPASGR

SEQ ID NO: 7 - protein, 11 aa, Artificial Sequence
  Description of Artificial Sequence Synthetic peptide
AGSDPLAGSLR

SEQ ID NO: 8 - protein, 11 aa, Artificial Sequence
  Description of Artificial Sequence Synthetic peptide
ADPGSLAGSLR

SEQ ID NO: 9 - protein, 357 aa, Homo sapiens
MSAIQAAWPSGTECIAKYNFHGTAEQDLPFCKGDVLTIVAVTKDPNWYKAKNKVGREGIIPANYVQKREGVKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASTPRTRASCQSSGQPLRP

SEQ ID NO: 10 - protein, 536 aa, Homo sapiens
MGSNKSKPKDASQRRRSLEPAENVHGAGGGAFPASQTPSKPASADGHRGPSAAFAPAAAEPKLFGGFNSSDTVTSPQRAGPLAGGVTTFVALYDYESRTETDLSFKKGERLQIVNNTEGDWWLAHSLSTGQTGYIPSNYVAPSDSIQAEEWYFGKITRRESERLLLNAENPRGTFLVRESETTKGAYCLSVSDFDNAKGLNVKHYKIRKLDSGGFYITSRTQFNSLQQLVAYYSKHADGLCHRLTTVCPTSKPQTQGLAKDAWEIPRESLRLEVKLGQGCFGEVWMGTWNGTTRVAIKTLKPGTMSPEAFLQEAQVMKKLRHEKLVQLYAVVSEEPIYIVTEYMSKGSLLDFLKGETGKYLRLPQLVDMAAQIASGMAYVERMNYVHRDLRAANILVGENLVCKVADFGLARLIEDNEYTARQGAKFPIKWTAPEAALYGRFTIKSDVWSFGILLTELTTKGRVPYPGMVNREVLDQVERGYRMPCPPECPESLHDLMCQCWRKEPEERPTFEYLQAFLEDYFTSTEPQYQPGENL

SEQ ID NO: 11 - protein, 13 aa, Artificial Sequence
  Description of Artificial Sequence Synthetic peptide
CGAGSDPLAGSLR

SEQ ID NO: 12 - protein, 10 aa, Artificial Sequence
  Description of Artificial Sequence Synthetic peptide
GSWFSGMCAR

SEQ ID NO: 13 - protein, 12 aa, Artificial Sequence
  Description of Artificial Sequence Synthetic peptide
YFMTSGCDPGGR

SEQ ID NO: 14 - protein, 12 aa, Artificial Sequence
  Description of Artificial Sequence Synthetic peptide
YFMTSGDPCGGR

SEQ ID NO: 15 - protein, 12 aa, Artificial Sequence
  Description of Artificial Sequence Synthetic peptide
YFMTSDPGCGGR

SEQ ID NO: 16 - protein, 12 aa, Artificial Sequence
  Description of Artificial Sequence Synthetic peptide
YFMTDPSGCGGR

SEQ ID NO: 17 - protein, 12 aa, Artificial Sequence
  Description of Artificial Sequence Synthetic peptide
YFMDPTSGCGGR

SEQ ID NO: 18 - protein, 19 aa, Artificial Sequence
  Description of Artificial Sequence Synthetic peptide
AGSLAGSLDPAGSLAGSLR

SEQ ID NO: 19 - DNA, 18 bp, Artificial Sequence
  Description of Artificial Sequence Synthetic oligonucleotide
gattagccacgtcgccgt

SEQ ID NO: 20 - DNA, 18 bp, Artificial Sequence
  Description of Artificial Sequence Synthetic oligonucleotide
gcatatagctagctctcg

SEQ ID NO: 21 - DNA, 22 bp, Artificial Sequence
  Description of Artificial Sequence Synthetic oligonucleotide
gacgacggcgacgtggctgcgc

SEQ ID NO: 22 - DNA, 18 bp, Artificial Sequence
  Description of Artificial Sequence Synthetic oligonucleotide
acggcgacgtggctaatc

SEQ ID NO: 23 - DNA, 11 bp, Artificial Sequence
  Description of Artificial Sequence Synthetic oligonucleotide
cgtcatcgtag

SEQ ID NO: 24 - protein, 15 aa, Artificial Sequence
  Description of Artificial Sequence Synthetic peptide
  MOD_RES (3)..(7): variable amino acid
  MOD_RES (10)..(14): variable amino acid
CFXXXXXDPXXXXXR

SEQ ID NO: 25 - protein, 35 aa, Artificial Sequence
  Description of Artificial Sequence Synthetic peptide
  MOD_RES (1)..(9): variable amino acid
  MOD_RES (12)..(21): variable amino acid
  MOD_RES (24)..(30): variable amino acid
  MOD_RES (32)..(35): variable amino acid
XXXXXXXXXDPXXXXXXXXXXDPXXXXXXXRXXXX

SEQ ID NO: 26 - protein, 34 aa, Artificial Sequence
  Description of Artificial Sequence Synthetic peptide
  MOD_RES (1)..(9): variable amino acid
  MOD_RES (12)..(21): variable amino acid
  MOD_RES (23)..(29): variable amino acid
  MOD_RES (31)..(34): variable amino acid
XXXXXXXXXDPXXXXXXXXXXFXXXXXXXRXXXX

SEQ ID NO: 27 - protein, 11 aa, Artificial Sequence
  Description of Artificial Sequence Synthetic peptide
AGSLAGSLDPR

SEQ ID NO: 28 - protein, 14 aa, Artificial Sequence
  Description of Artificial Sequence Synthetic peptide
CGWAGSDPLAGSLR

SEQ ID NO: 29 - protein, 14 aa, Artificial Sequence
  Description of Artificial Sequence Synthetic peptide
CGWAGSLDPAGSLR

SEQ ID NO: 30 - protein, 14 aa, Artificial Sequence
  Description of Artificial Sequence Synthetic peptide
CGWAGSLADPGSLR

SEQ ID NO: 31 - protein, 14 aa, Artificial Sequence
  Description of Artificial Sequence Synthetic peptide
CGWAGSLAGDPSLR

SEQ ID NO: 32 - protein, 14 aa, Artificial Sequence
  Description of Artificial Sequence Synthetic peptide
CGWAGSLAGSDPLR

SEQ ID NO: 33 - protein, 14 aa, Artificial Sequence
  Description of Artificial Sequence Synthetic peptide
RLSGADPLSGAWGC

SEQ ID NO: 34 - protein, 14 aa, Artificial Sequence
  Description of Artificial Sequence Synthetic peptide
GGGGGGGGDPGGGG

SEQ ID NO: 35 - protein, 14 aa, Artificial Sequence
  Description of Artificial Sequence Synthetic peptide
GGGGGGGDPGGGGG

SEQ ID NO: 36 - protein, 14 aa, Artificial Sequence
  Description of Artificial Sequence Synthetic peptide
GGGGGGDPGGGGGG

SEQ ID NO: 37 - protein, 14 aa, Artificial Sequence
  Description of Artificial Sequence Synthetic peptide
GGGGGDPGGGGGGG

SEQ ID NO: 38 - protein, 14 aa, Artificial Sequence
  Description of Artificial Sequence Synthetic peptide
GGGGDPGGGGGGGG

SEQ ID NO: 39 - protein, 14 aa, Artificial Sequence
  Description of Artificial Sequence Synthetic peptide
GGGGGGDPGGGGGG

SEQ ID NO: 40 - DNA, 18 bp, Artificial Sequence
  Description of Artificial Sequence Synthetic oligonucleotide
cgagagctagctatatgc

SEQ ID NO: 41 - protein, 15 aa, Artificial Sequence
  Description of Artificial Sequence Synthetic peptide
CCGGGGDPGGGGRIK

(19) United States
(12) Patent Application Publication
(10) Pub. No.: US 2008/0026480 A1
(43) Pub. Date: Jan. 31, 2008

(54) Detection systems for mass labels

(75) Inventor: Cesar E. Guerra, Guilford, CT (US)

Correspondence Address: WILMER, HALE, PERKIN & ELMER, LLP, 60 STATE STREET, BOSTON, MA, 02109, US

(73) Assignee: PerkinElmer LAS, Inc.

(21) Appl. No.: 11/706,500

(22) Filed: Feb. 14, 2007

Related U.S. Application Data
(60) Provisional application No. 60/773,512, filed on Feb. 14, 2006.

(51) Int. Cl. G01N 33/00; C07K 16/44

(52) U.S. Cl. 436/86; 530/387.1

(57) ABSTRACT

Disclosed are compositions and methods for sensitive detection of one or multiple analytes. The methods utilize special label components, referred to as reporter signals, that can be associated with, incorporated into, or otherwise linked to the analytes. The reporter signals can be altered such that the altered forms of different reporter signals can be distinguished from each other. Sets of reporter signals can be used where two or more of the reporter signals in a set have one or more common properties that allow the reporter signals having the common property to be distinguished and/or separated from other molecules lacking the common property. The reporter signal signals can be bound by the same specific binding molecule. Reporter signals can also be in conjunction with analytes, where no significant physical association between the reporter signals and analytes occurs; or alone, where no analyte is present.

### CROSS-REFERENCE TO RELATED APPLICATION

This application claims the benefit under 35 U.S.C. § 119(e) of U.S. Provisional Patent Application No. 60/773,512, filed on Feb. 14, 2006, entitled Detection Systems for Mass Labels, which is incorporated herein by reference in its entirety.

### FIELD OF THE INVENTION

This invention is generally in the field of detection of analytes and biomolecules, and more specifically in the field of multiplex detection and analysis of analytes and biomolecules.

### BACKGROUND OF THE INVENTION

Detection of molecules is an important operation in the biological and medical sciences. Such detection often requires the use of specialized label molecules, amplification of a signal, or both, because many molecules of interest are present in low quantities and do not, by themselves, produce detectable signals. Many labels, labeling systems, and signal amplification techniques have been developed. For example, nucleic acid molecules and sequences have been amplified and/or detected using polymerase chain reaction (PCR), ligase chain reaction (LCR), self-sustained sequence replication (3SR), nucleic acid sequence based amplification (NASBA), strand displacement amplification (SDA), and amplification with Qβ replicase (Birkenmeyer and Mushahwar, J. Virological Methods, 35:117-126 (1991); Landegren, Trends Genetics 9:199-202 (1993)). Proteins have been detected using antibody-based detection systems such as sandwich assays (Mailini and Maysef, “A sandwich method for enzyme immunoassay. I. Application to rat and human alpha-fetoprotein” J. Immunol. Methods 8:223-234 (1975)) and enzyme-linked immunosorbent assays (Engvall and Perlmann, “Enzyme-linked immunosorbent assay (ELISA). Quantitative assay of immunoglobulin” Immunochemistry 8:871-874 (1971)), and two-dimensional (2-D) gel electrophoresis (Patton, Biotechniques 28: 944-957 (2000)). Although these techniques are useful, most have significant drawbacks and limitations. For example, radioactive labels are dangerous and difficult to handle, fluorescent labels have limited capacity for multiplex detection because of limitations on distinguishable labels, and amplification methods can be subject to spurious signal amplification. There is a need for improved detection labels and detection techniques that can detect minute quantities of specific molecules and that can be highly multiplexed.

Analysis of protein expression and presence, such as proteome profiling or proteomics, requires sensitive detection of multiple proteins. Current methods in proteome profiling suggests that there is a shortage of tools necessary for such detection (Haynes and Yates, Proteome profiling-pitfalls and progress. Yeast 17(2):81-87 (2000)). While the techniques of chromatography and capillary electrophoresis are amenable to proteomic studies and have seen significant development efforts (see for example, Krull et al., Specific applications of capillary electrochromatography to biopolymers, including proteins, nucleic acids, peptide mapping, antibodies, and so forth. J Chromatogr A, 887:137-63 (2000), Hage, Affinity chromatography: a review of clinical applications. Clin Chem, 45(5):593-615 (1999), Hage et al., Chromatographic Immunoassays., Anal Chem, 73(07): 198 A-205 A, (2001), Krull et al., Labeling reactions applicable to chromatography and electrophoresis of minute amounts of proteins. J Chromatogr B Biomed Sci Appl, 699:173-208 (1997)), the workhorse of the industry remains two dimensional electrophoresis where the two dimensions are isoelectric focusing and molecular size. Haynes and Yates point out the significant shortcomings of the technique but discuss the utility of the method in light of such shortcomings. Hayes and Yates also discuss the techniques of Isotope Coded Affinity Tags (ICAT), LC-LC-MS/MS, and stable isotope labeling techniques (Shevchenko et al., Rapid ‘de novo’peptide sequencing by a combination of nanoelectrospray, isotopic labeling and a quadrupole/time-of-flight mass spectrometer. Rapid Commun Mass Spectrom 11(9):1015-1024 (1997); Oda et al., Accurate quantitation of protein expression and site-specific phosphorylation. Proc Natl Acad Sci USA 96(12):6591-6596 (1999)).

Aebersold et al. (WO 00/11208) have described labels of the composition PRG-L-A, where PRG is a protein reactive group, L is a linker (that may contain isotopically distinguishable composition), and A is an affinity moiety. Aebersold et al. describes a method where the protein reactive group is used to attach the label to a protein, an affinity capture molecule is used to capture the affinity moiety, the remaining proteins are discarded, then the affinity moiety is released and the labeled proteins are detected by mass spectrometry. The method of Aebersold et al. does not involve fragmentation or other modification of the labels or proteins.

The technique of ICAT, where cysteine residues are labeled with heavy or light tags that each contain affinity moieties, in control and tester samples, has received significant interest and holds potential for protein profiling (Gygi et al., Quantitative analysis of complex protein mixtures using isotope-coded affinity tags. Nat. Biotechnol. 17(10):994-999 (1999), Griffin et al., Quantitative proteomic analysis using a MALDI quadrupole time-of-flight mass spectrometer, Anal. Chem., 73:978-986 (2001)). Gygi et al. and Griffin et al. have demonstrated relative profiling of two protein samples, where the two samples are distinguished utilizing linkers containing either eight normal hydrogen or eight heavy hydrogen (deuterium) atoms. The relative concentrations of labeled proteins are determined by ratio of peaks that are separated by the corresponding 8 amu difference in the linker molecules. Current implementations have been limited to two labels. This technique does not involve fragmentation or other modification of the labels or proteins.

Mass spectrometry has been used to detect phosphorylated proteins (DeGnore and Qin, Fragmentation of phosphopeptides in an ion trap mass spectrometer. J. Am. Soc. Mass Spectrom. 9:1175-1188 (1998); Qin and Chait, Identification and characterization of posttranslational modifications of proteins by MALDI ion trap mass spectrometry. Anal Chem, 69:4002-9 (1997); Annan et al., A multidimensional electrospray MS-based approach to phosphopeptide mapping. Anal. Chem. 73:393-404 (2001)). The methods make use of a signature mass to indicate the presence of a phosphate group, for example m/z=63 and/or m/z=79 corresponding to PO₂⁻ and PO₃⁻ ions in negative ion mode, or the neutral loss of 98 Daltons from the parent ion indicates the loss of H₃PO₄ from the phosphorylated peptide, indicate phosphorylated Ser, Tyr, Thr. Once phosphorylated amino acids are identified, the peptide containing the modification is sequenced by standard MS/MS techniques. There is a need for a high reliability, highly multiplexed readout system for proteomics.

The status of any living organism may be defined, at any given time in its lifetime, by the complex constellation of proteins that constitute its “proteome.” While the complete status of the proteome could be defined by listing all proteins present (including modified variants) as well as their intracellular locations and concentrations, such a task is beyond the capabilities of any current single analytical method. However, attempts have been made to define the status of a cell or tissue by identifying and measuring the relative concentrations of a small subset of proteins. For example, Conrads et al., Analytical Chemistry, 72:3349-3354 (2000), have described the use of “Accurate Mass Tags” (AMT) for proteome-wide protein identification. Conrads et al. show, for a simple organism, that a mass spectrometer of sufficient mass accuracy and resolution can be used to detect certain tryptic digest fragments from proteins. Once identified, the AMTs may be directly detected in samples by tryptic digest of the proteins, and high accuracy, high resolution mass spectrometry.

While the concept of Accurate Mass Tags is useful for protein discovery, as well as for generating peptide patterns in conventional biological experiments, it does not solve the problem of sensitivity that is at the heart of a truly useful diagnostic multi-protein assessment. A useful assessment consisting of AMTs will require samples containing a minimum of 2000 to 10,000 cells in order to permit reliable readout. This is so because many important cellular proteins are present at levels of only 500 to 5000 molecules per cell. If a clinically relevant protein is present in 500 copies per cell, and a precious clinical sample from a cancer patient contains only 1000 cells, the total number of proteins is 500,000, an amount that lies below the limit of detection by conventional mass spectrometry. Thus, the types of measurements proposed by Conrads et al. for the study of proteomes after identification of AMTs are not suitable for addressing important clinical problems such as the diagnosis of cancer.

### BRIEF SUMMARY OF THE INVENTION

In a first aspect, the invention provides a specific binding molecule (e.g., an antibody) that is specific for a plurality of reporter signals in a set of reporter signals, wherein the set of reporter signals comprises a plurality of reporter signals wherein the reporter signals have a common property, wherein the common property allows the reporter signals to be distinguished and/or separated from molecules lacking the common property and wherein the reporter signals can be altered, wherein the altered forms of each reporter signal can be distinguished from every other altered form of reporter signal.

In some embodiments of all of the aspects of the invention, the reporter signals are peptides, oligonucleotides, carbohydrates, polymers, oligopeptides, or peptide nucleic acids. In some embodiments, each reporter signal of the set is associated with, or coupled to, a different specific binding molecule.

In some embodiments of all of the aspects of the invention, the reporter signals are associated with, or coupled to, decoding tags, wherein each reporter signal is associated with, or coupled to, a different decoding tag. In some embodiments, the reporter signals comprise peptides. In some embodiment, the peptides have the same mass-to-charge ratio, the same amino acid composition, or the same amino acid sequence. In some embodiments, each peptide contains a different distribution of heavy isotopes or a different distribution of substituent groups. In some embodiments, the peptides each have a different amino acid sequence.

In some embodiments of all of the aspects of the invention, each the reporter signals has a labile or scissile bond in a different location than the other reporter signals in the set. In some embodiments, each the reporter signals has a labile or scissile bond in the same location as the other reporter signals in the set. In some embodiments, the labile or scissile bond is a covalent bond aspartic acid residue and proline residue.

In another aspect, the invention provides a method comprising (a) separating, detecting, sorting, or immobilizing a set of reporter signals by binding a plurality of the reporter signals in the set to a specific binding molecule, (b) separating the set of reporter signals, where each reporter signal has a common property, from molecules lacking the common property, (c) altering the reporter signals, and (d) detecting and distinguishing the altered forms the reporter signals from each other. In some embodiments, each of the reporter signals is attached (or coupled) to an analyte, such as a protein.

In some embodiments of all of the aspects of the invention, the reporter signals are attached or coupled (e.g., covalently or non-covalently) to analytes, such as proteins, peptides, carbohydrates, peptidoglycan, lipids, and glycoproteins. In some embodiments, the common property allows the reporter signal-coupled analytes to be distinguished or separated from molecules lacking the common property.

In some embodiments of all of the aspects of the invention, the common property is mass-to-charge ratio, wherein the reporter signals are altered by altering their mass, wherein the altered forms of the reporter signals can be distinguished via differences in the mass-to-charge ratio of the altered forms of reporter signals. In some embodiments, the reporter signals (or the masses thereof) are altered by fragmentation. In certain embodiments, alteration of the reporter signals also alters their charge.

In some embodiments of all of the aspects of the invention, the reporter signals are altered by cleavage or fragmentation at a photocleavable amino acid. In certain embodiments, the reporter signals are fragmented at an aspartic acid-proline bond, a methionine, or a phosphorylated amino acid.

In particular embodiments of all of the aspects of the invention, the set of reporter signals comprises two or more, three or more, four or more, five or more, six or more, seven or more, eight or more, nine or more, ten or more, twenty or more, thirty or more, forty or more, fifty or more, sixty or more, seventy or more, eighty or more, ninety or more, or one hundred or more different reporter signals.

In some embodiments of all of the aspects of the invention, the reporter signals are peptides, oligonucleotides, carbohydrates, polymers, oligopeptides, or peptide nucleic acids. In some embodiments, the reporter signals are associated with, or coupled to, specific binding molecules, wherein each reporter signal is associated with, or coupled to, a different specific binding molecule. In certain embodiments, the reporter signals are associated with, or coupled to, decoding tags, wherein each reporter signal is associated with, or coupled to, a different decoding tag.

In particular embodiments, the reporter signals comprise peptides, wherein the peptides have the same mass-to-charge ratio. In some embodiments, the peptides (i.e., the reporter signal peptides) have the same amino acid composition or have the same amino acid sequence. In some embodiments, each peptide contains a different distribution of heavy isotopes or contains a different distribution of substituent groups. In certain embodiments, each peptide has a different amino acid sequence.

In various embodiments of all of the aspects of the invention, the reporter signals are coupled to the proteins or peptides. In particular embodiments, the common property is not an affinity tag. In some embodiments, one or more affinity tags are associated with the reporter signals.

In a further aspect, the invention provides a method comprising first associating the reporter signals with one or more analytes, wherein each reporter signal is associated with, or coupled to, a different specific binding molecule, wherein each specific binding molecule can interact specifically with a different one of the analytes, wherein the reporter signals are associated with the analytes via interaction of the specific binding molecules with the analytes, and then (a) separating, detecting, sorting, or immobilizing a set of reporter signals by binding a plurality of the reporter signals in the set to a specific binding molecule, (b) separating the set of reporter signals, where each reporter signal has a common property, from molecules lacking the common property, (c) altering the reporter signals, and (d) detecting and distinguishing the altered forms the reporter signals from each other. In some embodiments, the analytes are proteins or peptides (e.g., proteins or peptides from each of one or more samples). In some embodiments, different sets of reporter signals are associated with different samples

In some embodiments of all of the aspects of the invention, the reporter signals are associated with a single sample. In some embodiments, the sample is produced by a separation procedure, wherein the separation procedure comprises liquid chromatography, gel electrophoresis, two-dimensional chromatography, two-dimensional gel electrophoresis, isoelectric focusing, thin layer chromatography, centrifugation, filtration, ion chromatography, immunoaffinity chromatography, membrane separation, or a combination of these.

In various embodiments of all of the aspects of the invention, steps (a) through (d) are repeated one or more times using a different set of reporter signals each time. In some embodiments, the different sets of reporter signals each comprise the same reporter signals. In certain embodiments, the different samples are obtained from the same protein sample, are obtained at different times, are obtained from the same type of organism, are obtained from the same type of tissue, are obtained from the same organism, are obtained at different times, are obtained from different organisms, are obtained from different types of tissues, are obtained from different strains or different species of organisms, or are obtained from different cellular compartments.

In various embodiments of all of the aspects of the invention, the methods further comprise identifying or preparing proteins or peptides corresponding to proteins or peptides present in one sample but not present in another sample. In some embodiments, the methods of the invention further comprise determining the relative amount of proteins or peptides in the different samples.

In some embodiments of all of the aspects of the invention, the sets of reporter signals each contain a single reporter signal. In certain embodiments, not all of the reporter signals in the set are distinguished or separated from molecules lacking the common property, not all of the reporter signals are altered, and/or not all of the altered forms of the reporter signals are detected at the same time. In various embodiments, all of the reporter signals in the set are distinguished or separated from molecules lacking the common property, all of the reporter signals are altered, and all of the altered forms of the reporter signals are detected at different times.

In some embodiments of all of the aspects of the invention, steps (a) through (d) of the methods are performed separately for each reporter signal. In some embodiments, the altered forms of the labeled proteins detected collectively constitute a catalog of proteins. In some embodiments, steps (c) and (d) are performed simultaneously.

In particular embodiments of all of the aspects of the invention, the altered forms of the target protein fragments are detecting using mass spectrometry. In certain embodiments, steps (b) through (d) are performed with a tandem mass spectrometer. In some embodiments, the tandem mass spectrometer comprises a first stage and a last stage, wherein step (b) is performed using the first stage of the tandem mass spectrometer to select ions in a narrow mass-to-charge range, wherein step (c) is performed by collision with a gas, and wherein step (d) is performed using the final stage of the tandem mass spectrometer. In certain embodiments, the first stage of the tandem mass spectrometer is a quadrupole mass filter. In certain embodiments, the final stage of the tandem mass spectrometer is a time of flight analyzer. In some embodiments, the mass-to-charge range is varied to cover the mass-to-charge ratio of each of the target protein fragments.

In another aspect, the invention provides a kit comprising (a) a set of reporter molecules, wherein each reporter molecule comprises a reporter signal, wherein the reporter signals have a common property, wherein the common property allows the reporter signals to be distinguished or separated from molecules lacking the common property, wherein the reporter signals can be altered, wherein the altered forms of each reporter signal can be distinguished from every other altered form of reporter signal, wherein each different reporter molecule comprises a different decoding tag and a different reporter signal, and (b) a specific binding molecule that is specific for a plurality of the reporter signals. In some embodiments, the kit further comprises a decoding tag. In some embodiments, the kit further comprises a set of coding molecules, wherein each coding molecule comprises a specific binding molecule and a coding tag, wherein each specific binding molecule can interact specifically with a different analyte, wherein each coding tag can interact specifically with a different decoding tag. In particular embodiments, the common property is the same mass-to-charge ratio.

In another aspect, the invention provides a kit comprising a set of reporter molecules and a specific binding molecule that is specific for a plurality of the reporter signals, wherein each reporter molecule comprises a reporter signal and a coupling tag, wherein the reporter signals have a common property, wherein the common property allows the reporter signals to be distinguished or separated from molecules lacking the common property, wherein the reporter signals can be altered, wherein the altered forms of each reporter signal can be distinguished from every other altered form of reporter signal, and wherein each different reporter molecule comprises a different coupling tag and a different reporter signal.

In another aspect, the invention provides a set of labeled proteins wherein each labeled protein comprises a protein or peptide and a reporter signal attached to the protein or peptide, wherein the labeled proteins have a common property, wherein the common property allows the labeled proteins comprising the same protein or peptide to be distinguished or separated from molecules lacking the common property, wherein the reporter signals can be altered, wherein the altered forms of each reporter signal can be distinguished from every other altered form of reporter signal, and wherein alteration of the reporter signals alters the labeled proteins, wherein altered forms of each labeled protein can be distinguished from every other altered form of labeled protein.

In a further aspect, the invention provides a labeled protein wherein the labeled protein comprises a protein or peptide and a reporter signal attached to the protein or peptide, wherein the labeled protein has a common property, wherein the common property allows the labeled protein to be distinguished or separated from molecules lacking the common property, wherein a plurality of the reporter signals can be bound by the same specific binding molecule, wherein the reporter signal can be altered, and wherein alteration of the reporter signals alters the labeled protein, wherein altered form of the labeled protein can be distinguished from the unaltered form of labeled protein.

In some embodiments of all of the aspects of the invention, the steps of the methods are repeated one or more times using a different set of reporter signals each time. In some embodiments of the methods of the invention, prior to step (a), the different sets of reporter signals are attached to proteins or peptides in different samples. In some embodiments, the different sets of reporter signals each comprise the same reporter signals.

In certain embodiments, the sets of reporter signals each contain a single reporter signal. In some embodiments, the pattern of the presence, amount, presence and amount, or absence of labeled proteins in one of the samples constitutes a catalog of proteins in the sample. In particular embodiments, the pattern of the presence, amount, presence and amount, or absence of labeled proteins in a second one of the samples constitutes a catalog of proteins in the second sample, wherein the catalog of proteins in the first sample is a first catalog and the catalog of proteins in the second sample is a second catalog. In some embodiments, the methods of the invention further comprising comparing the first catalog and the second catalog.

In another aspect, the invention provides a method comprising (a) separating, detecting, sorting, or immobilizing a set of reporter signals by binding a plurality of the reporter signals to a specific binding molecule, (b) altering the reporter signals, and (c) detecting and distinguishing the altered forms of the reporter signals from each other. In some embodiments, each of the reporter signals is attached to an analyte.

In yet another aspect, the invention provides a method of detecting a protein or peptide, the method comprising (a) separating, detecting, sorting, or immobilizing a labeled protein by binding the labeled protein to a specific binding molecule, (b) altering the labeled protein, wherein the labeled protein comprises a protein or peptide and a reporter signal attached to the protein or peptide, wherein the labeled protein is altered by altering the reporter signal, and (c) detecting and distinguishing the altered form of the labeled protein from the unaltered form of labeled protein. In some embodiments, the method further comprises detecting the unaltered form of labeled protein.

In a further aspect, the invention provides a method of detecting a protein comprising detecting a labeled protein, wherein the labeled protein comprises a protein or peptide and a reporter signal attached to the protein or peptide, wherein the labeled protein is altered by altering the reporter signal, wherein a plurality of the reporter signal peptides can be bound by the same specific binding molecule, detecting an altered form of the labeled protein, wherein the labeled protein is altered by altering the reporter signal, and identifying the protein based on the characteristics of the labeled protein and altered form of the labeled protein. In certain embodiments, the labeled protein and altered form of the labeled protein are detected by detecting the mass-to-charge ratio of the labeled protein and the mass-to-charge ratio of the altered form of the labeled protein or the mass-to-charge ratio of the altered form of the reporter signal.

In various embodiments of all of the aspects of the invention, the one or more labeled proteins are derived from a single sample. In various embodiments, a single labeled protein is distinguished or separated from other molecules. In certain embodiments, a plurality of labeled proteins are distinguished or separated from other molecules. In certain embodiments, the detected altered forms of the labeled proteins constitute a catalog of proteins in the sample. In certain embodiments, one or more labeled proteins are derived from each of a plurality of samples. In some embodiments, a single labeled protein derived from each of the samples is distinguished or separated from other molecules. In certain embodiments, a plurality of labeled proteins derived from each of the samples are distinguished or separated from other molecules. In certain embodiments, the detected altered forms of the labeled proteins derived from each sample constitute a catalog of proteins in the sample.

In another aspect, the invention provides a catalog of proteins and peptides comprising proteins and peptides in one or more samples detected by (a) separating, detecting, sorting, or immobilizing one or more labeled proteins by binding the labeled proteins to a specific binding molecule, (b) separating one or more of the labeled proteins from other molecules, wherein the labeled proteins are derived from the one or more samples, wherein each labeled protein comprises a protein or peptide and a reporter signal attached to the protein or peptide, (c) altering the reporter signals, thereby altering the labeled proteins, and (d) detecting and distinguishing the altered forms the labeled proteins from each other.

In yet another aspect, the invention provides a set of nucleic acid molecules wherein each nucleic acid molecule comprises a nucleotide segment encoding an amino acid segment comprising a reporter signal peptide and a protein or peptide of interest, wherein the reporter signal peptides have a common property, wherein the common property allows the reporter signal peptides to be distinguished or separated from molecules lacking the common property, wherein a plurality of the reporter signal peptides can be bound by the same specific binding molecule, and wherein the reporter signal peptides can be altered, wherein the alteration of the reporter signal peptides alters the reporter signal peptides or alters the amino acid segments, wherein the altered form of each reporter signal peptide or each altered amino acid segment can be distinguished from the altered forms of the other reporter signal peptides or other amino acid segments.

In various embodiments of all of the aspects of the invention, the proteins or peptides of interest of each amino acid segment is different or is the same. In some embodiments, the proteins or peptides of interest of at least two amino acid segments are different or are the same. In some embodiments, the proteins or peptides of interest are related, are produced in the same cascade, are expressed under the same conditions, are associated with the same disease, are associated with the same cell type or same tissue type, or are in the same enzymatic pathway.

In some embodiments of all of the aspects of the invention, the nucleotide segment encodes a plurality of amino acid segments each comprising a reporter signal peptide and a protein or peptide of interest. In some embodiments, the protein or peptide of interest of the amino acid segments in each of the nucleotide segments are different. In some embodiments, the protein or peptide of interest of at least two of the amino acid segments in each of the nucleotide segments are different. In certain embodiments, the set consists of a single nucleic acid molecule, wherein the nucleic acid molecule comprises a plurality of nucleotide segments each encoding an amino acid segment. In further embodiments, the amino acid segment comprises a cleavage site (e.g., a self-cleaving segment) near or at the junction between the reporter signal peptide and the protein or peptide of interest. In some embodiments, the cleavage site is a trypsin cleavage site. In some embodiments, the cleavage site is cleaved.

In another aspect, the invention provides a set of nucleic acid molecules wherein each nucleic acid molecule comprises a nucleotide segment encoding an amino acid segment comprising a reporter signal peptide and a protein or peptide of interest, wherein the amino acid segments each comprise an amino acid subsegment, wherein each amino acid subsegment comprises a portion of the protein or peptide of interest and all or a portion of the reporter signal peptide, wherein a plurality of the reporter signal peptides can be bound by the same specific binding molecule, wherein the amino acid subsegments have a common property, wherein the common property allows the amino acid subsegments to be distinguished or separated from molecules lacking the common property, and wherein the reporter signal peptides can be altered, wherein the altered form of each reporter signal peptide can be distinguished from the altered forms of the other reporter signal peptides.

In another aspect, the invention provides a set of nucleic acid molecules wherein each nucleic acid molecule comprises a nucleotide segment encoding an amino acid segment comprising a reporter signal peptide and a protein or peptide of interest, wherein the amino acid segments each comprise an amino acid subsegment, wherein each amino acid subsegment comprises a portion of the protein or peptide of interest and all or a portion of the reporter signal peptide, wherein the amino acid subsegments have a common property, wherein the common property allows the amino acid subsegments to be distinguished or separated from molecules lacking the common property, wherein a plurality of the reporter signal peptides can be bound by the same specific binding molecule, wherein the reporter signal peptides can be altered, wherein alteration of the reporter signal peptides alters the amino acid subsegments, wherein the altered form of each amino acid subsegment can be distinguished from the altered forms of the other amino acid subsegments.

In yet another aspect, the invention provides a set of amino acid segments wherein each amino acid segment comprises a reporter signal peptide and a protein or peptide of interest, wherein the reporter signal peptides have a common property, wherein the common property allows the reporter signal peptides to be distinguished or separated from molecules lacking the common property, wherein a plurality of the reporter signal peptides can be bound by the same specific binding molecule, wherein the reporter signal peptides can be altered, wherein the altered form of each reporter signal peptide can be distinguished from the altered forms of the other reporter signal peptides.

In various embodiments, the amino acid segment is a protein or peptide. In some embodiments, the set consists of a single amino acid segment, wherein the amino acid segment comprises a plurality of reporter signal peptides.

In another aspect, the invention provides a cell an organism comprising a set of nucleic acid molecules wherein each nucleic acid molecule comprises a nucleotide segment encoding an amino acid segment comprising a reporter signal peptide and a protein or peptide of interest, wherein the reporter signal peptides have a common property, wherein the common property allows the reporter signal peptides to be distinguished or separated from molecules lacking the common property, wherein a plurality of the reporter signal peptides can be bound by the same specific binding molecule, and wherein the reporter signal peptides can be altered, wherein the altered form of each reporter signal peptide can be distinguished from the altered forms of the other reporter signal peptides.

In another aspect, the invention provides a set of cells or organisms, wherein each cell or each organism comprises a nucleic acid molecule wherein each nucleic acid molecule comprises a nucleotide segment encoding an amino acid segment comprising a reporter signal peptide and a protein or peptide of interest, wherein the reporter signal peptides have a common property, wherein the common property allows the reporter signal peptides to be distinguished or separated from molecules lacking the common property, wherein a plurality of the reporter signal peptides can be bound by the same specific binding molecule, and wherein the reporter signal peptides can be altered, wherein the altered form of each reporter signal peptide can be distinguished from the altered forms of the other reporter signal peptides.

In some embodiments of all of the aspects of the invention, each cell or each organism further comprises additional nucleic acid molecules. In some embodiments, the set consists of a single cell or a single organism, wherein the cell or organism comprises a plurality of nucleic acid molecules. In some embodiments, the set consists of a single cell or a single organism, wherein the cell or organism comprises a set of nucleic acid molecules, wherein the set of nucleic acid molecules consists of a single nucleic acid molecule, wherein the nucleic acid molecule encodes a plurality of nucleic acid segments.

In another aspect, the invention provides a method of detecting expression, the method comprising detecting a target altered reporter signal peptide derived from one or more expression samples, wherein the one or more expression samples collectively comprise a set of nucleic acid molecules, wherein each nucleic acid molecule comprises a nucleotide segment encoding an amino acid segment comprising a reporter signal peptide and a protein or peptide of interest, wherein the reporter signal peptides have a common property, wherein the common property allows the reporter signal peptides to be distinguished or separated from molecules lacking the common property, wherein a plurality of the reporter signal peptides can be bound by the same specific binding molecule, wherein the reporter signal peptides can be altered, wherein either (a) the altered form of each reporter signal peptide can be distinguished from the altered forms of the other reporter signal peptides, wherein the target altered reporter signal peptide is one of the altered reporter signal peptides, or (b) alteration of the reporter signal peptides alters the amino acid segments, wherein the altered form of each amino acid segment can be distinguished from the altered forms of the other amino acid segments, wherein the target altered amino acid segment is one of the altered amino acid segments, and wherein detection of the target altered reporter signal peptide indicates expression of the amino acid segment (or the nucleotide segment encoding the amino acid segment) that comprises the reporter signal peptide from which the target altered reporter signal peptide or the target amino acid segment is derived.

In some embodiments of all of the aspects of the invention, the method further comprises determining the amount of the target altered reporter signal peptide detected, wherein the amount of the target altered reporter signal peptide indicates the amount present in the one or more expression samples of the amino acid segment that comprises the reporter signal peptide from which the target altered reporter signal peptide is derived. In certain embodiments, the amount of the amino acid segment present is proportional to the amount of the target altered reporter signal peptide detected.

In some embodiments of all of the aspects of the invention, the method further comprises detecting a plurality of the altered reporter signal peptides, wherein detection of each altered reporter signal peptide indicates expression of the amino acid segment that comprises the reporter signal peptide from which that altered reporter signal peptide is derived. In certain embodiments, the method further comprises determining the amount of the altered reporter signal peptides detected, wherein the amount of each altered reporter signal peptide indicates the amount present in the one or more expression samples of the amino acid segment that comprises the reporter signal peptide from which that altered reporter signal peptide is derived. In some embodiments, the amount of the amino acid segment present is proportional to the amount of the altered reporter signal peptide detected. In particular embodiments, the presence, absence, amount, or presence and amount of the altered forms of the reporter signal peptides indicates the presence, absence, amount, or presence and amount in the expression sample of the reporter signal peptides from which the altered forms of the reporter signal peptides are derived, wherein the presence, absence, amount, or presence and amount of the reporter signal peptides in the expression sample constitutes a protein signature of the expression sample. In some embodiments, the altered forms of the reporter signal peptides are detecting using mass spectrometry (e.g., using a tandem mass spectrometer). In some embodiments, the mass spectrometer includes a quadrupole set for single-ion filtering, a collision cell, and a time-of-flight spectrometer.

In certain embodiments of all of the aspects of the invention, the invention further provides comparing the protein signature to one or more other protein signatures. In some embodiments, the detected altered reporter signal peptides are derived from a plurality of expression samples. In some embodiments, the methods of the invention further comprise identifying differences between the protein signatures produced from the expression samples and the control expression sample. In some embodiments, the differences are differences in the presence, amount, presence and amount, or absence of reporter signal peptides in the expression samples and the control expression sample.

In further embodiments of all of the aspects of the invention, the plurality of expression samples comprises a control expression sample and a tester expression sample, wherein the tester expression sample, or the source of the tester expression sample, is treated so as to destroy, disrupt or eliminate one or more of the amino acid segments in the tester expression sample and wherein the reporter signal peptides corresponding to the destroyed, disrupted, or eliminated amino acid segments will be produced from the control expression sample but not the tester expression sample. In some embodiments, the tester expression sample is treated so as to destroy, disrupt or eliminate one or more of the amino acid segments in the tester expression sample. In some embodiments, treatment of the tester expression sample (or its source) is accomplished by exposing cells from which the tester sample will be derived with a compound, composition, or condition that will reduce or eliminate expression of one or more of the nucleotide segments.

In some embodiments of all of the aspects of the invention, one or more of the amino acid segments in the tester sample are eliminated by separating the one or more of the amino acid segments from the tester expression sample. In some embodiments, the one or more of the amino acid segments are separated by affinity separation. In some embodiments, the methods of the invention further comprise identifying differences in the reporter signal peptides in the control expression sample and tester expression sample. In some embodiments, the methods of the invention further comprise identifying differences between the reporter signal peptides in the expression samples. In certain embodiments, at least two of the expression samples, or the sources of the at least two expression samples, are subjected to different conditions (such as exposure to different compounds, or exposure to compound and no exposure to compound). In some embodiments, the sources of the expression samples are cells. In some embodiments, differences in the protein signatures of the at least two expression samples indicate the effect of the different conditions.

In various embodiments of all of the aspects of the invention, the methods further comprise producing a second protein signature from a second expression sample and comparing the first protein signature and second protein signature, wherein differences in the first and second protein signatures indicate differences in source or condition of the source of the first and second expression samples. In some embodiments, the methods further comprise producing a second protein signature from a second expression sample and comparing the first protein signature and second protein signature, wherein differences in the first and second protein signatures indicate differences in protein modification of the first and second expression samples. In some embodiments, the second expression sample is a sample from the same type of cells as the first expression sample except that the cells from which the first expression sample is derived are modification-deficient relative to the cells from which the second expression sample is derived. In some embodiments, the second expression sample is a sample from a different type of cells than the first expression sample, and wherein the cells from which the first expression sample is derived are modification-deficient relative to the cells from which the second expression sample is derived. In some embodiments, the expression sample is derived from one or more cells.

In certain embodiments of all of the aspects of the invention, the protein signature indicates the physiological state of the cells or indicates the effect of a treatment of the cells. In some embodiments, the cells are derived from an organism, wherein the cells are treated by treating the organism. In some embodiments, the organism (e.g., a human) is treated by administering a compound to the organism.

In certain embodiments of all of the aspects of the invention, altered reporter signal peptides are detected in a first and a second expression sample. In some embodiments, the second expression sample is a sample obtained from the same type of organism, the same type of tissue, the same organism, a different organism, a different type of tissue, a different species of organism, a different strain of organism, or a different cellular compartment as the first expression sample. In some embodiments, the second expression sample is obtained at a different time than the first expression sample.

In various embodiments of all of the aspects of the invention, the methods further comprise altering the reporter signal peptides, separating the reporter signal peptides from the expression samples, cleaving the reporter signal peptides from the proteins or peptides of interest, cleaving the amino acid segments into a reporter signal peptide portion and a protein portion, and/or mixing two or more of the expression samples together, wherein the mixed amino acid segments were derived from two or more different expression samples. In some embodiments, the reporter signal peptides are distinguished or separated from the expression samples or from the proteins or peptides of interest based on the common property.

In some embodiments of all of the aspects of the invention, expression of the amino acid segment that comprises the reporter signal peptide from which the target altered reporter signal peptide is derived identifies the expression sample from which the target altered reporter signal peptide is derived. In certain embodiments, the expression samples are derived from one or more cells, wherein expression of the amino acid segment that comprises the reporter signal peptide from which the target altered reporter signal peptide is derived identifies the cell from which the identified expression sample is derived. In certain embodiments, the expression samples are derived from one or more organisms (e.g., human), tissues, cells, or cell lines, wherein expression of the amino acid segment that comprises the reporter signal peptide from which the target altered reporter signal peptide is derived identifies the organism, tissue, cell, or cell line from which the identified expression sample is derived.

In some embodiments of all of the aspects of the invention, the expression sequences of at least two nucleic acid molecules are different or are the same. In some embodiments, the expression of the amino acid segment or the nucleic acid molecule is induced. In some embodiments, the nucleic acids molecules are produced by replicating nucleic acids in one or more nucleic acid samples.

In various embodiments of all of the aspects of the invention, each amino acid segment further comprises an epitope tag. In some embodiments, the epitope tag of each amino acid segment is different or the same. In some embodiments, the epitope tag of at least two amino acid segments is different or the same. In particular embodiments, the amino acid segments are distinguished or separated from the one or more expression samples via the epitope tags. In some embodiments, the reporter signal peptide of each amino acid segment is different or is the same.

In certain embodiments of all of the aspects of the invention, the nucleic acid molecules are in cells, cell lines, or organisms. For example, each nucleic acid molecule may be in a different cell (or cell line or organism) or may be in the same cell (or cell line or organism). In certain embodiments, each nucleic acid molecule further comprises expression sequences, wherein the expression sequences are operably linked to the nucleotide segment such that the amino acid segment can be expressed. In some embodiments, the expression sequences comprise translation expression sequences and/or transcription expression sequences. In some embodiments, the amino acid segment is expressed in vitro, in vivo, or in cell culture. In some embodiments, the expression sequences of each nucleic acid molecule are different, are differently regulated, are the same, or are similarly regulated. In some embodiments, a plurality of the expression sequences are expression sequences of, or derived from, genes expressed as part of the same expression cascade.

In some embodiments of all of the aspects of the invention, each nucleic acid molecule further comprises replication sequences, wherein the replication sequences allow replication of the nucleic acid molecules. In some embodiments, the nucleic acid molecules can be replicated in vitro, in vivo, or in cell culture. In some embodiments, the nucleic acids molecules are produced by replicating nucleic acids in one or more nucleic acid samples. In some embodiments, the nucleic acids are replicated using pairs of primers, wherein each of the first primers in the primer pairs used to produce the nucleic acid molecules comprises a nucleotide sequence encoding the reporter signal peptide. In some embodiments, each first primer further comprises expression sequences. In particular embodiments, the nucleotide sequence of each first primer also encodes an epitope tag.

In certain embodiments of all of the aspects of the invention, the reporter signal peptide of at least two amino acid segments are different or the same. In some embodiments, the nucleic acid molecules are in cells of an organism. The nucleic acid molecules may be in substantially all of the cells of the organism, or may be in some of the cells of the organism. The amino acid segments may be expressed in substantially all of the cells of the organism or may be expressed in some of the cells of the organism.

In further embodiments of all of the aspects of the invention, each nucleic acid molecule further comprises integration sequences, wherein the integration sequences allow integration of the nucleic acid molecules into other nucleic acids. In particular embodiments, the nucleic acid molecules are integrated into a chromosome of the cell, cell line, or organism. For example, the nucleic acid molecules may be integrated into the chromosome at a predetermined location. In some embodiments, the chromosome is an artificial chromosome. In some embodiments, the nucleic acid molecules are, or are integrated into, a plasmid.

In some embodiments of all of the aspects of the invention, the expression samples are produced from the cells, the cell lines, or the organisms. In some embodiments, each expression sample is produced from cells from a cell sample or organism, wherein each expression sample is produced from a different cell sample or organism. In some embodiments, each cell sample is subjected to a different condition, is brought into contact with a different test compound, is cultured under different conditions, is derived from a different organism, or is derived from a different tissue. In some embodiments, each cell sample is taken from the same source at different times. In particular embodiments, the expression samples are produced by lysing the cells.

In some embodiments of all of the aspects of the invention, the reporter signal peptide is distinguished or separated from the peptide or protein of interest. In some embodiments, a plurality of different altered reporter signal peptides are detected, wherein detection of each altered reporter signal peptide indicates expression of the amino acid segment that comprises the reporter signal peptide from which that altered reporter signal peptide is derived. In some embodiments, different expression samples comprise different nucleic acid molecules, wherein detection of each altered reporter signal peptide indicates expression in the expression sample that comprises the nucleic acid molecule that comprises the nucleotide segment encoding the amino acid segment that comprises the reporter signal peptide from which that altered reporter signal peptide is derived. In some embodiments, there are a plurality of different expression samples, wherein each different expression sample comprises different nucleic acid molecules, wherein detection of an altered reporter signal peptide indicates expression in the expression sample that comprises the nucleic acid molecule that comprises the nucleotide segment encoding the amino acid segment that comprises the reporter signal peptide from which the detected altered reporter signal peptide is derived.

In another aspect, the invention features a method of detecting expression comprising detecting an altered amino acid subsegment derived from one or more expression samples, wherein the one or more expression samples collectively comprise a set of nucleic acid molecules, wherein each nucleic acid molecule comprises a nucleotide segment encoding an amino acid segment comprising a reporter signal peptide and a protein or peptide of interest, wherein the amino acid segments each comprise an amino acid subsegment, wherein each amino acid subsegment comprises a portion of the protein or peptide of interest and all or a portion of the reporter signal peptide, wherein the amino acid subsegments have a common property, wherein the common property allows the amino acid subsegments to be distinguished or separated from molecules lacking the common property, wherein a plurality of the reporter signal peptides can be bound by the same specific binding molecule, wherein the reporter signal peptides can be altered, wherein alteration of the reporter signal peptides alters the amino acid subsegments, wherein the altered form of each amino acid subsegment can be distinguished from the altered forms of the other amino acid subsegments, wherein the target altered amino acid subsegment is one of the altered amino acid subsegments, wherein detection of the target altered amino acid subsegment indicates expression of the amino acid segment from which the target altered amino acid subsegment is derived.

In another aspect, the invention provides a method of detecting cells or cell samples, the method comprising detecting a target altered reporter signal peptide derived from one or more cells or cell samples, wherein the one or more cells or cell samples collectively comprise a set of nucleic acid molecules, wherein each nucleic acid molecule comprises a nucleotide segment encoding an amino acid segment comprising a reporter signal peptide and a protein or peptide of interest, wherein the reporter signal peptides have a common property, wherein the common property allows the reporter signal peptides to be distinguished or separated from molecules lacking the common property, wherein a plurality of the reporter signal peptides can be bound by the same specific binding molecule, wherein the reporter signal peptides can be altered, wherein either (a) the altered form of each reporter signal peptide can be distinguished from the altered forms of the other reporter signal peptides, wherein the target altered reporter signal peptide is one of the altered reporter signal peptides or (b) alteration of the reporter signal peptides alters the amino acid segments, wherein the altered form of each amino acid segment can be distinguished from the altered forms of the other amino acid segments, wherein the target altered amino acid segment is one of the altered amino acid segments, and wherein detection of the target altered reporter signal peptide or target altered amino acid segment indicates the presence of the cell or cell sample from which the target altered reporter signal peptide or target altered amino acid segment is derived.

In various embodiments of all of the aspects of the invention, each cell or cell sample is engineered to contain at least one of the nucleic acid molecules, wherein the reporter signal peptide of the amino acid segment encoded by the nucleotide segment of the nucleic acid molecule in each cell or cell sample is different. In some embodiments, each cell or cell sample having a trait of interest comprises the same reporter signal peptide. In certain embodiments, the trait of interest is a heterologous gene. In some embodiments, the heterologous gene comprises the nucleic acid molecule or encodes the amino acid segment.

In some embodiments of all of the aspects of the invention, a plurality of different altered reporter signal peptides are detected, wherein detection of each altered reporter signal peptide indicates the presence of the cell or the cell sample from which that altered reporter signal peptide is derived. In some embodiments, different cells or cell samples comprise different nucleic acid molecules, wherein detection of each altered reporter signal peptide indicates the presence of the cell or the cell sample that comprises the nucleic acid molecule that comprises the nucleotide segment encoding the amino acid segment that comprises the reporter signal peptide from which that altered reporter signal peptide is derived. In some embodiments, there are a plurality of different cells or cell samples, wherein each different cell or cell sample comprises different nucleic acid molecules, wherein detection of an altered reporter signal peptide indicates the presence of the cell or cell sample that comprises the nucleic acid molecule that comprises the nucleotide segment encoding the amino acid segment that comprises the reporter signal peptide from which the detected altered reporter signal peptide is derived.

In another aspect, the invention provides a method of detecting cells comprising detecting an altered amino acid subsegment derived from one or more cells, wherein the one or more cells collectively comprise a set of nucleic acid molecules, wherein each nucleic acid molecule comprises a nucleotide segment encoding an amino acid segment comprising a reporter signal peptide and a protein or peptide of interest, wherein the amino acid segments each comprise an amino acid subsegment, wherein each amino acid subsegment comprises a portion of the protein or peptide of interest and all or a portion of the reporter signal peptide, wherein the amino acid subsegments have a common property, wherein the common property allows the amino acid subsegments to be distinguished or separated from molecules lacking the common property, wherein a plurality of the reporter signal peptides can be bound by the same specific binding molecule, wherein the reporter signal peptides can be altered, wherein alteration of the reporter signal peptides alters the amino acid subsegments, wherein the altered form of each amino acid subsegment can be distinguished from the altered forms of the other amino acid subsegments, wherein the target altered amino acid subsegment is one of the altered amino acid subsegments, wherein detection of the target altered amino acid subsegment indicates the presence of the cell from which the target altered amino acid subsegment is derived.

In yet another aspect, the invention provides a method of detecting organisms, comprising detecting a target altered reporter signal peptide derived from one or more organisms, wherein the one or more organisms collectively comprise a set of nucleic acid molecules, wherein each nucleic acid molecule comprises a nucleotide segment encoding an amino acid segment comprising a reporter signal peptide and a protein or peptide of interest, wherein the reporter signal peptides have a common property, wherein the common property allows the reporter signal peptides to be distinguished or separated from molecules lacking the common property, wherein a plurality of the reporter signal peptides can be bound by the same specific binding molecule, wherein the reporter signal peptides can be altered, wherein either (a) the altered form of each reporter signal peptide can be distinguished from the altered forms of the other reporter signal peptides, wherein the target altered reporter signal peptide is one of the altered reporter signal peptides, or (b) alteration of the reporter signal peptides alters the amino acid segments, wherein the altered form of each amino acid segment can be distinguished from the altered forms of the other amino acid segments, wherein the target altered amino acid segment is one of the altered amino acid segments, and wherein detection of the target altered reporter signal peptide or the target altered amino acid segment indicates the presence of the organism from which the target altered reporter signal peptide or the target altered amino acid segment is derived.

In some embodiments of all of the aspects of the invention, each organism is engineered to contain at least one of the nucleic acid molecules, wherein the reporter signal peptide of the amino acid segment encoded by the nucleotide segment of the nucleic acid molecule in each organism is different. In some embodiments, each organism having a trait of interest, such as a transgene, comprises the same reporter signal peptide. In some embodiments, the transgene comprises the nucleic acid molecule or encodes the amino acid segment.

In some embodiments of all of the aspects of the invention, a plurality of different altered reporter signal peptides are detected, wherein detection of each altered reporter signal peptide indicates the presence of the organism from which that altered reporter signal peptide is derived. In some embodiments, different organisms comprise different nucleic acid molecules, wherein detection of each altered reporter signal peptide indicates the presence of the organism that comprises the nucleic acid molecule that comprises the nucleotide segment encoding the amino acid segment that comprises the reporter signal peptide from which that altered reporter signal peptide is derived. In some embodiments, there are a plurality of different organisms, wherein each different organism comprises different nucleic acid molecules, wherein detection of an altered reporter signal peptide indicates the presence of the organism that comprises the nucleic acid molecule that comprises the nucleotide segment encoding the amino acid segment that comprises the reporter signal peptide from which the detected altered reporter signal peptide is derived.

In yet another aspect, the invention provides a method of detecting organisms, comprising detecting an altered amino acid subsegment derived from one or more organisms, wherein the one or more organisms collectively comprise a set of nucleic acid molecules, wherein each nucleic acid molecule comprises a nucleotide segment encoding an amino acid segment comprising a reporter signal peptide and a protein or peptide of interest, wherein the amino acid segments each comprise an amino acid subsegment, wherein each amino acid subsegment comprises a portion of the protein or peptide of interest and all or a portion of the reporter signal peptide, wherein the amino acid subsegments have a common property, wherein the common property allows the amino acid subsegments to be distinguished or separated from molecules lacking the common property, wherein a plurality of the reporter signal peptides can be bound by the same specific binding molecule, wherein the reporter signal peptides can be altered, wherein alteration of the reporter signal peptides alters the amino acid subsegments, wherein the altered form of each amino acid subsegment can be distinguished from the altered forms of the other amino acid subsegments, wherein the target altered amino acid subsegment is one of the altered amino acid subsegments, wherein detection of the target altered amino acid subsegment indicates the presence of the organism from which the target altered amino acid subsegment is derived.

In yet another aspect, the invention provides a method comprising (a) associating one of a plurality of reporter signals with one or more analytes in each of a plurality of samples, wherein each reporter signal has a common property, wherein the common property allows each reporter signal to be separated from molecules lacking the common property, (b) separating, detecting, sorting, or immobilizing one or more of the reporter signals by binding the reporter signals to a specific binding molecule, (c) separating the analytes contained in each sample, (d) altering the reporter signals, and (e) detecting the altered forms the reporter signals. In some embodiments, the method further comprises, following step (a) and prior to step (b), combining two or more of the samples.

In some embodiments of all of the aspects of the invention, analytes in each sample are associated with only one reporter signal, wherein the reporter signal associated with analytes in each sample is different. In some embodiments, the analytes are separated by contact with a capture array.

The disclosed method has advantageous properties which can be used as a detection system in a number of fields, including antibody or protein microarrays, DNA microarrays, expression profiling, comparative genomics, immunology, diagnostic assays, and quality control.

It is an object of the present invention to provide a method for the multiplexed determination of presence and/or amount of analytes.

It is another object of the present invention to provide a method of detecting, binding, separating and/or sorting labels and/or labeled analytes using antibodies or other specific binding molecules that can bind the labels.

It is an object of the present invention to provide labeled proteins such that the presence, amount, or presence and amount of the proteins can be determined.

It is another object of the present invention to provide a method for labeling proteins so as to allow the multiplexed determination of presence, amount, or presence and amount of proteins.

It is another object of the present invention to provide a method for the multiplexed determination of presence, amount, or presence and amount of proteins.

It is an object of the present invention to provide a method for detecting a mass tag signature.

It is an object of the present invention to provide a method for detecting a protein signature.

It is another object of the present invention to provide an assessment of the identity and purity of the peptides comprising a protein signature.

It is another object of the present invention to provide a method for detecting phosphopeptides, or other posttranslational protein modifications, among the peptides comprising a protein signature.

It is another object of the present invention to provide kits for generating mass tag signatures.

It is another object of the present invention to provide kits for generating protein signatures.

### BRIEF DESCRIPTION OF THE DRAWINGS

FIGS. 1A and 1B are graphs of mass-to-charge ratio (m/z) versus signal intensity. FIG. 1A shows the results where there is no fragmentation of the reporter signal. A single peak represents the parent ion. FIG. 1B shows the results where the reporter signal is fragmented. The parent ion along with two fragmentation ions are detected.

FIGS. 2A and 2B are graphs of mass-to-charge ratio (m/z) versus detected counts. FIG. 2A shows the results where no fragmentation of reporter signals A and B occurs. FIG. 2B shows the results where all of the reporter signals are fragmented (A fragments to A1 and A2, B fragments to B1 and B2).

FIG. 3 is an example of an ESI-TOF mass spectrum of an example of a reporter signal peptide (LAT3838 in this case). Most of the complexity of the spectrum comes from fragmentation of the reporter signal peptide in the source.

FIG. 4 is an example a spectrum of a selected reporter signal peptide (LAT3838 in this case) following fragmentation. The parent reporter signal was selected at a filter setting of m/z=1044 and altered by fragmented by collision with argon gas at about 20 eV collision energy. The daughter reporter signal peptide fragment at m/z=600 corresponds to the expected PAGSLR⁺ fragment (amino acids 6-11 of SEQ ID NO:2).

FIG. 5 is an example of a spectrum of the fragmentation products of five reporter signal peptides (LAT3838 and LAT3843 through LAT3856). The peaks corresponding to the reporter signal peptide fragments of each are labeled.

FIG. 6 is an example of a spectrum showing the effect of the loss of a phosphate group from reporter signal peptide fragments.

FIG. 7 is an example of a spectrum showing differentiation of reporter signal peptide fragments based on the use of stable isotopes in the reporter signal peptides.

FIG. 8 is an example of a spectrum of a complex mixture of molecules that includes five reporter signal peptides (m/z=1389.6).

FIGS. 9A and 9B are an example of a spectrum of a set of reporter signal peptides following selection based on mass-to-charge ratio (m/z around 1390) and illustrating a nearly non-existent background everywhere except at m/z around 1390. This is the same sample (prior to selection) from which the complex spectrum of FIG. 8 was obtained. FIG. 9B is an expanded view of the peak. Selection was not perfect in this example as the finite resolution of the filter allowed three peaks to pass.

FIG. 10 is an example of a spectrum of the fragmentation products of the selected reporter signal peptides of FIG. 9 (originally in the complex sample of FIG. 8). Five prominent peaks of approximately the same magnitude appear at the expected m/z of the reporter signal peptide fragments of each of the five reporter signal peptides. Unfragmented reporter signal peptides remain in the rightmost peak (m/z near 1390).

FIG. 11 is a graph of mass spectrometry peaks of fragmented mass labels of Table 8 before separation using an antibody to the mass labels. The low mass signal peaks are the five highest peaks between 401 and 632 amu and the high mass signal peaks are the five highest peaks between 1360 and 1589 amu.

FIG. 12 is a graph of mass spectrometry peaks of fragmented mass labels of Table 8 after separation using an antibody to the mass labels. Only two of the mass labels were separated by the antibody. The low mass signal peaks are the two highest peaks between 401 and 632 amu and the high mass signal peaks are the two highest peaks between 1360 and 1589 amu.

FIGS. 13A and 13B are graphs of mass spectrometry peaks of fragmented mass labels of Table 7 before separation using an antibody to the mass labels. In FIG. 13A, the low mass signal peaks are the peaks around 451 amu and the high mass signal peaks are the peaks around 1483 amu. FIG. 13B is an expansion of the high mass signal area showing the seven high mass signal peaks at 1474, 1477, 1480, 1483, 1486, 1489, AND 1492 amu.

FIGS. 14A and 14B are graphs of mass spectrometry peaks of fragmented mass labels of Table 7 after separation using an antibody to the mass labels. All seven of the mass labels were separated by the antibody. In FIG. 14A, the low mass signal peaks are the peaks around 451 amu and the high mass signal peaks are the peaks around 1483 amu.

FIG. 14B is an expansion of the high mass signal area showing the seven high mass signal peaks at 1474, 1477, 1480, 1483, 1486, 1489, AND 1492 amu.

### DETAILED DESCRIPTION OF THE INVENTION

All of the references cited herein are hereby each incorporated in their entirety.

Disclosed are compositions and methods for sensitive detection of one or multiple analytes. In general, the methods involve the use of special label components, referred to as reporter signals, that can be associated with, incorporated into, or otherwise linked to the analytes. Reporter signals can also be used merely in conjunction with analytes, with no significant association between the analytes and reporter signals. The reporter signals can be detected, bound, separated and/or sorted using antibodies or other specific binding molecules that can bind the reporter signals. Compositions where reporter signals are associated with, incorporated into, or otherwise linked to the analytes are referred to as reporter signal/analyte conjugates. Such conjugates include reporter signals associated with analytes, such as a reporter signal probe hybridized to a nucleic acid sequence; reporter signals covalently coupled to analytes, such as reporter signals linked to proteins via a linking group; and reporter signals incorporated into analytes, such as fusions between a protein of interest and a peptide reporter signal.

In some embodiments, the reporter signals can be altered such that the altered forms of different reporter signals can be distinguished from each other. Reporter signal/analyte conjugates can be altered, generally through alteration of the reporter signal portion of the conjugate, such that the altered forms of different reporter signals, altered forms of different reporter signal/analyte conjugates, or both, can be distinguished from each other. Where the reporter signal or reporter signal/analyte conjugate is altered by fragmentation, any, some, or all of the fragments can be distinguished from each other, depending on the embodiment. For example, where reporter signals fragmented into two parts, either or both parts of the reporter signals can be distinguished. Where reporter signal/analyte conjugates are fragmented into two parts (with the break point in the reporter signal portion), either the reporter signal fragment, the reporter signal/analyte fragment, or both can be distinguished. In some embodiments, only one part of a fragmented reporter signal will be detected and so only this part of the reported signals need be distinguished. The altered reporter signals can be detected, bound, separated and/or sorted using antibodies or other specific binding molecules that can bind the reporter signals.

In some embodiments, sets of reporter signals can be used where two or more of the reporter signals in a set have one or more common properties that allow the reporter signals having the common property to be distinguished and/or separated from other molecules lacking the common property. In other embodiments, sets of reporter signal/analyte conjugates can be used where two or more of the reporter signal/analyte conjugates in a set have one or more common properties that allow the reporter signal/analyte conjugates having the common property to be distinguished and/or separated form other molecules lacking the common property. In still other embodiments, analytes can be fragmented (prior to or following conjugation) to produce reporter signal/analyte fragment conjugates (which can be referred to as fragment conjugates). In such cases, sets of fragment conjugates can be used where two or more of the fragment conjugates in a set have one or more common properties that allow the fragment conjugates having the common property to be distinguished and/or separated form other molecules lacking the common property. It should be understood that fragmented analytes can be considered analytes in their own right. In this light, reference to fragmented analytes is made for convenience and clarity in describing certain embodiments and to allow reference to both the base analyte and the fragmented analyte.

As indicated above, reporter signals conjugated with analytes can be altered while in the conjugate and distinguished. Conjugated reporter signals can also be dissociated or separated, in whole or in part, from the conjugated analytes prior to their alteration. Where the reporter signals are dissociated (in whole or in part) from the analytes, the method can be performed such that the fact of association between the analyte and reporter signal is part of the information obtained when the reporter signal is detected. In other words, the fact that the reporter signal may be dissociated from the analyte for detection does not obscure the information that the detected reporter signal was associated with the analyte.

The reporter signals can be separated from the analytes using antibodies or other specific binding molecules that can bind the reporter signals. This can be, for example, an additional step or manipulation; as, or as part of, the separation step in various forms of the disclosed methods; or as, or as part of, the detection step in various forms of the disclosed method. Thus, for example, the common property can be used for the separation step or operation while binding of reporter signals to a specific binding molecule can be used to separate reporter signals (and molecules to which they are attached or bound) to be separated or sorted prior to use of the common property to separate components having the common property form other components. Alternatively, the common property can be the capability of a specific binding molecule to bind reporter signal(s). By using, for example, a specific binding molecule that can bind the reporter signals in a set of reporter signals (or all of the reporter signals being used in a given assay), the reporter signals can be separated from other components and materials that may be present. This can allow, for example, much cleaner detection and/or analysis of reporter signals.

Reporter signals can also be in conjunction with analytes (such as in mixtures of reporter signals and analytes), where no significant physical association between the reporter signals and analytes occurs; or alone, where no analyte is present. In such cases, where reporter signals are not or are no longer associated with analytes, sets of reporter signals can be used where two or more of the reporter signals in a set have one or more common properties that allow the reporter signals having the common property to be distinguished and/or separated from other molecules lacking the common property.

Detection of the reporter signals indicates the presence of the corresponding analytes. The reporter signals can have two key features. First, the reporter signals can be used in sets where all the reporter signals in the set have similar properties. The similar properties allow the reporter signals to be distinguished and/or separated from other molecules lacking one or more of the properties. In some embodiments, the reporter signals in a set have the same mass-to-charge ratio (m/z). That is, the reporter signals in a set are isobaric. This allows the reporter signals to be separated precisely from other molecules based on mass-to-charge ratio. The result of the filtering is a huge increase in the signal to noise ratio (S/N) for the system, allowing more sensitive and accurate detection. Alternatively, or in addition, reporter signals can be used in sets such that the resulting labeled analytes will have similar properties allowing the labeled analytes to be distinguished and/or separated from other molecules lacking one or more of the properties. The reporter signals (and/or the analytes attached thereto) can be detected, bound, separated and/or sorted using antibodies or other specific binding molecules that can bind the reporter signals (and/or the attached analytes) based on these and/or other similar properties.

Second, all the reporter signals in a set can be fragmented, decomposed, reacted, derivatized, or otherwise modified to distinguish the different reporter signals in the set.

The reporter signals of the invention include those described in Chait et al., U.S. Pat. No. 6,824,981 (hereby incorporated by reference). The reporter signals may be detected using mass spectrometry which allows sensitive distinctions between molecules based on their mass-to-charge ratios. The disclosed reporter signals can be used as general labels in myriad labeling and/or detection techniques. A set of isobaric reporter signals can be used for multiplex labeling and/or detection of many analytes since the reporter signal fragments can be designed to have a large range of masses, with each mass individually distinguishable upon detection.

Current technologies are limited in their ability to multiplex labels. In contrast, the disclosed method allows the readout of many samples simultaneously and high internal accuracy in comparison to a sequential readout system.

A. Reporter Molecule Labeling

In one form of the disclosed method, referred to as reporter molecule labeling (RML), reporter signals are associated with analytes to be detected and/or quantitated. In some embodiments, the reporter signals can be dissociated from the analytes prior to detection. The reporter signals may be associated with the analytes via interaction of specific binding molecules with the analytes. The reporter signals are either directly or indirectly associated with the specific binding molecules such that interaction of the specific binding molecules with the analytes allows the reporter signals to be associated with the analytes. For example, a reporter signal can be associated with a specific binding molecule that interacts with the analyte of interest. The specific binding molecule in the reporter molecule interacts with the analyte thus associating the reporter signal with the analyte. The method can be performed such that the fact of association between the analyte and reporter signal is part of the information obtained when the reporter signal is detected. In other words, the fact that the reporter signal may be dissociated from the analyte for detection does not obscure the information that the detected reporter signal was associated with the analyte.

The disclosed method increases the sensitivity and accuracy of detection of an analyte of interest. Non-limiting forms of the disclosed method make use of multistage detection systems to increase the resolution of the detection of molecules having very similar properties. The method involves at least two stages. The first stage is filtration or selection that allows passage or selection of reporter signals (that is, a subset of the molecules present), based upon intrinsic properties of the reporter signals (and their attached analytes), and discrimination against all other molecules. The subsequent stage(s) further separate(s) and/or detect(s) the reporter signals which were filtered in the first stage. A key facet of this method is that a multiplexed set of reporter signals (and/or their attached analytes) will be selected by the filter and subsequently cleaved, decomposed, reacted, or otherwise modified to realize the identities and/or quantities of the reporter signals and/or fragmented labeled (i.e., attached) analytes in further stages. There is a correspondence between the specific binding molecule or reporter signal and the detected daughter fragment(s).

B. Reporter Signal Labeling

In another form of the disclosed method, referred to as reporter signal labeling, reporter signals are used for sensitive detection of one or multiple analytes. In the method, analytes labeled with reporter signals are analyzed using the reporter signals to distinguish the labeled analytes (where the analytes are labeled with the reporter signals). Analytes (e.g., proteins) are detected by detecting a reporter signal, labeled analyte, or both; or by distinguishing different reporter signals, different labeled analytes, or both. Detection of the reporter signals indicates the presence of the corresponding analyte(s) (where the analytes are labeled with (i.e., attached to) the reporter signals. Thus, reporter signal labeling is a general technique for labeling, detection, and quantitation of analytes.

The detected analyte(s) can then be analyzed using known techniques. The labels provide a unique analyte/label composition that can specifically identify the analyte(s). This is accomplished through the use of the reporter signals as the labels. The labeled analyte(s) can be fragmented (e.g., by digestion with an enzyme, such as a protease or a lipidase, depending on the analyte) prior to analysis. An analyte sample to be analyzed can also be subjected to fractionation or separation to reduce the complexity of the samples. For example, the labeled proteins or reporter signal fusions can be separated and/or sorted using antibodies or other specific binding molecules that can bind the reporter signals. Fragmentation and fractionation can also be used together in the same assay. Such fragmentation and fractionation can simplify and extend the analysis of the analytes. The analyte/label composition (including those where the analyte has been fragmented) can be detected, bound, separated and/or sorted using antibodies or other specific binding molecules that can bind the reporter signals.

The disclosed method can be used in many modes. For example, the disclosed method can be used to detect a specific analyte (in a specific sample or in multiple samples) or multiple analytes (in a single sample or multiple samples). In each case, the analyte(s) to be detected can be separated either from other, unlabeled analytes or from other molecules lacking a property of the labeled analyte(s) to be detected. For example, analytes in a sample can be generally labeled with reporter signals and some analytes can be separated on the basis of some property of the analytes. For example, the separated analytes could have a certain mass-to-charge ratio (separation based on mass-to-charge ratio will select both labeled and unlabeled analytes having the selected mass-to-charge ratio). As another example, all of the labeled analytes can be distinguished and/or separated from unlabeled molecules based on a feature of the reporter signal such as an affinity tag. Where different affinity tags are used, some labeled analytes can be distinguished and/or separated from others. Reporter signal labeling allows profiling of analytes and cataloging of analytes. As another example, labeled analytes can be detected, bound, separated and/or sorted using antibodies or other specific binding molecules that can bind the reporter signals.

In one mode of the disclosed method, multiple analytes in multiple samples are labeled where all of the analytes in a given sample are labeled with the same reporter signal. That is, the reporter signal is used as a general label of the analytes in a sample. Each sample, however, uses a different reporter signal. This allows samples as a whole to be compared with each other. By additionally separating or distinguishing different analytes in the samples, one can easily analyze many analytes in many samples in a single assay. For example, proteins in multiple samples can be labeled with reporter signals as described above, and the samples mixed together. If some or all of the various labeled proteins are separated by, for example, association of the proteins with antibodies on an array, the presence and amount of a given protein in each of the samples can be determined by identifying the reporter signals present at each array element. If the protein corresponding to a given array element was present in a particular sample, then some of the protein associated with that array element will be labeled with the reporter signal used to label that particular sample. Detection of that reporter signal will indicate this. This same relationship holds true for all of the other samples. Further, the amount of reporter signal detected can indicate the amount of a given protein in a given sample, and the simultaneous quantitation of protein in multiple samples can provide a particularly accurate comparison of the levels of the proteins in the various samples.

Reporter signals can be coupled or directly associated with an analyte. For example, a reporter signal can be coupled to an analyte via reactive groups, or a reporter molecule (composed of a specific binding molecule and a reporter signal) can be associated with an analyte. The reporter signals can be attached to analytes in any manner. For example, reporter signals can be covalently coupled to proteins through a sulfur-sulfur bond between a cysteine on the protein and a cysteine on the reporter signal. Many other chemistries and techniques for coupling compounds to analytes are known and can be used to couple reporter signals to analytes. For example, coupling can be made using thiols, epoxides, nitrites for thiols, NHS esters, isothiocyanates for amines, and alcohols for carboxylic acids. Reporter signals can be attached to analytes either directly or indirectly, for example, via a linker.

Reporter signals, or constructs containing reporters signals, also can be attached or coupled to analytes by ligation. Methods for ligation of nucleic acids are well known (see, for example, Sambrook et al. Molecular Cloning: A Laboratory Manual, second edition, 1989, Cold Spring Harbor Laboratory Press, New York.), and efficient protein ligation is known (see, for example, Dawson et al., “Synthesis of proteins by native chemical ligation” Science 266, 776-9 (1994); Hackeng et al., “Chemical synthesis and spontaneous folding of a multidomain protein: anticoagulant microprotein S” Proc Natl Acad Sci USA 97:14074-8 (2000); Dawson et al., “Synthesis of Native Proteins by Chemical Ligation” Ann. Rev. Biochem. 69:923-960 (2000); U.S. Pat. No. 6,184,344; PCT Publication WO 98/28434).

Alternatively, a reporter signal can be associated with an analyte indirectly. In this mode, a “coding” molecule containing a specific binding molecule and a coding tag can be associated with the analyte (via the specific binding molecule). Alternatively, a coding tag can be coupled or directly associated with the analyte. Then a reporter signal associated with a decoding tag (such a combination is another form of reporter molecule) is associated with the coding molecule through an interaction between the coding tag and the decoding tag. An example of this interaction is hybridization where the coding and decoding tags are complementary nucleic acid sequences. The result is an indirect association of the reporter signal with the analyte. This mode has the advantage that all of the interactions of the reporter signals with the coding molecule can be made chemically and physically similar by using the same types of coding tags and decoding tags for all of the coding molecules and reporter molecules in a set.

Reporter signals can be fragmented, decomposed, reacted, derivatized, or otherwise modified, for example, in a characteristic way. This allows an analyte to which the reporter signal is attached to be identified by the correlated detection of the labeled analyte and/or one or more of the products of the labeled analyte following fragmentation, decomposition, reaction, derivatization, or other modification of the reporter signal (the labeled analyte is the analyte/reporter signal combination). The analyte can also be identified by the correlated detection of the reporter signal fusion and one or more of the products of the reporter signal fusion following fragmentation, decomposition, reaction, derivatization, or other modification of the reporter signal peptide. The alteration of the reporter signal will alter the labeled analyte in a characteristic and detectable way. Together, the detection of a characteristic labeled analyte and a characteristic product of the labeled analyte or a characteristic product of (that is, altered form on a reporter signal fusion can uniquely identify the analyte (although the altered form alone can be detected, if desired). In this way, using the disclosed method and materials, one or more analytes (or the expression thereof) can be detected, either alone or together (for example, in a multiplex assay). Further, one or more analytes (or expression thereof) in one or more samples can be detected in a multiplex manner.

In some embodiments, the reporter signals are fragmented to yield fragments of similar charge but different mass. This allows each labeled analyte (and/or each reporter signal) in a set to be distinguished by the different mass-to-charge ratios of the fragments of the reporter signals. This is possible since, although the unfragmented reporter signals in a set are isobaric, the fragments of the different reporter signals are not. For example, the fragments of the different reporter signals can be designed to have different mass-to-charge ratios. In the disclosed method, this allows each analyte/reporter signal combination to be distinguished by the mass-to-charge ratios of the analyte/reporter signals after fragmentation of the reporter signal. Thus, a key feature of the disclosed reporter signals is that the reporter signals have a similarity of properties while the modified reporter signals are distinguishable.

Analytes can be detected using the disclosed reporter signals in a variety of ways. For example, the analyte and attached reporter signal can be detected together, one or more fragments of the analyte and the attached reporter signal(s) can be detected together, the fragments of the reporter signal can be detected, or a combination. One non-limiting form of detection involves detection of the reporter signal and/or the analyte/reporter signal both before and after fragmentation of the reporter signal.

A non-limiting form of the disclosed method involves correlated detection of the reporter signals both before and after fragmentation of the reporter signal. This allows labeled analytes to be detected and identified via the change in labeled analyte. That is, the nature of the reporter signal detected (non-fragmented versus fragmented) identifies the analyte as labeled. Where the analytes and reporter signals are detected by mass-to-charge ratio, the change in mass-to-charge ratio between fragmented and non-fragmented samples provides the basis for comparison. Such mass-to-charge ratio detection is accomplished, for example, with mass spectrometry.

Although reference is made above and elsewhere herein to detection of a “protein” or “proteins,” the disclosed method and compositions encompass proteins, peptides, and fragments of proteins or peptides. Thus, reference to a protein herein is intended to refer to proteins, peptides, and fragments of proteins or peptides unless the context clearly indicates otherwise. As used herein “labeled protein” refers to a protein, peptide, or fragment of a protein or peptide to which a reporter signal is attached unless the context clearly indicates otherwise. The labeled protein(s) can be fragmented, such as by protease digestion, prior to analysis. A protein sample to be analyzed can also be subjected to fractionation or separation to reduce the complexity of the samples. For example, the labeled proteins can be separated and/or sorted using antibodies or other specific binding molecules that can bind the reporter signals. Fragmentation and fractionation can also be used together in the same assay. Such fragmentation and fractionation can simplify and extend the analysis of the proteins.

Reporter signals can be attached to proteins either directly or indirectly, for example, via a linker. Reporter signals also can be attached to proteins by ligation (for example, protein ligation of a reporter signal peptide to a protein).

It is possible to form labeled proteins where the reporter signal is specifically attached to phosphopeptides. Chemistry for specific derivatization of phosphoserine or phosphotyrosine residues has been described (Zhou et al. A systematic approach to the problem of protein phosphorylation, Nat. Biotech. 19:375-378 (2001), Oda et al., Enrichment analysis of phosphorylated proteins as a tool for probing the phosphoproteome, Nat. Biotech. 19:379-382 (2001)). Tryptic peptides treated according to either of these two protocols will display reactive sulfhydryls at sites of protein phosphorylation. These sites may be reacted with reporter signals to generate a labeled protein. Non-phosphorylated peptides will not be derivatized.

Not all labeled protein fragments that can be made in the disclosed method from a protein sample will be unique. Because some proteins have common motifs that may be identical in different proteins, some protein fragments or peptides produced from a sample will be identical although they were derived from different proteins. For example, some families of related proteins have such common motifs or common amino acid sequences. Thus, in some embodiments of the disclosed method, detection of a characteristic labeled protein may be the result of detection of a common portion of related proteins. Such a result can be an advantage when detection of the family of proteins is desired. Alternatively, such collective detection of related proteins can be avoided by focusing on detection of unique fragments (that is, non-identical portions) of the proteins in the family. For convenience, as used herein, detection of a common portion of multiple related proteins is intended to be encompassed by reference to detection of a unique protein, labeled protein, or other component, unless the context clearly indicates otherwise.

A powerful form of the disclosed method is use of proteins labeled with reporter signals to assay multiple samples (for example, time series assays or other comparative analyses). Knowledge of the temporal response of a biological system following perturbation is a very powerful process in the pursuit of understanding the system. To follow the temporal response a sample of the system is obtained (for example, cells from a cell culture, mice initially synchronized and sacrificed) at determined times following the perturbation. Knowledge of spatial protein profiles (for example, relative position within a tissue section) is a very powerful process in the pursuit of understanding the biological system.

In the disclosed method a series of samples can each be labeled with a different reporter signal from a set of reporter signals. Non-limiting reporter signals for this purpose would be those using differentially distributed mass. In particular, stable isotopes may be used to ensure that members of the set of reporter signals would behave chemically identically and yet would be distinguishable. Exemplary sets of labels could be as shown in Tables 1, 7 and 8. The asterisk represents a heavy form of the residue. In Table 1, one heavy isotope, such as ¹³C or ¹⁵N, is present. In Table 7, ¹³C and ¹⁵N are present in the heavy residues. The labels of Table 1 could be used, for example, where each of five time points could be labeled with one of the five indicated labels and the mixture of the samples could be read out simultaneously. In Table 1, the unfragmented labels are SEQ ID NO:1 and the fragmented labels are amino acids 7-12 of SEQ ID NO:1. In Table 7, the unfragmented labels are SEQ ID NOs:34-38 (top to bottom) and the fragmented labels are amino acids 10-14 of SEQ ID NO:34, 9-14 of SEQ ID NO:35, 8-14 of SEQ ID NO:36, 7-14 of SEQ ID NO:37, and 6-14 of SEQ ID NO:38. In Table 8, the unfragmented labels are SEQ ID NO:39 and the fragmented labels are amino acids 8-14 of SEQ ID NO:39.

TABLE 1

| | Mass | Fragment | Fragment mass |
|---|---|---|---|
| Sequence | (amu) | Sequence | (amu) |
| CG*G*G*G*DPGGGGR | 949 | PGGGGR | 499 |
| CG*G*G*GDPGGGG*R | 949 | PGGGG*R | 500 |
| CG*G*GGDPGGG*G*R | 949 | PGGG*G*R | 501 |
| CG*GGGDPGG*G*G*R | 949 | PGG*G*G*R | 502 |
| CGGGGDPG*G*G*G*R | 949 | PG*G*G*G*R | 503 |

TABLE 7

| | | | Fragment |
|---|---|---|---|
| | Mass | Fragment | mass |
| Sequence | (amu) | Sequence | (amu) |
| G*G*G*G*G*G*DPGGGGGG | 933 | PGGGGGG | 458 |
| G*G*G*G*G*GDPG*GGGGG | 933 | PG*GGGGG | 461 |
| G*G*G*G*GGDPG*G*GGGG | 933 | PG*G*GGGG | 464 |
| G*G*G*GGGDPG*G*G*GGG | 933 | PG*G*G*GGG | 467 |
| G*G*GGGGDPG*G*G*G*GG | 933 | PG*G*G*G*GG | 470 |
| G*GGGGGDPG*G*G*G*G*G | 933 | PG*G*G*G*G*G | 473 |
| GGGGGGDPG*G*G*G*G*G* | 933 | PG*G*G*G*G*G* | 476 |

TABLE 8

| | Mass | Fragment | Fragment mass |
|---|---|---|---|
| Sequence | (amu) | Sequence | (amu) |
| GGGGGGGGDPGGGG | 915 | PGGGG | 344 |
| GGGGGGGDPGGGGG | 915 | PGGGGG | 401 |
| GGGGGGDPGGGGGG | 915 | PGGGGGG | 458 |
| GGGGGDPGGGGGGG | 915 | PGGGGGGG | 515 |
| GGGGDPGGGGGGGG | 915 | PGGGGGGGG | 572 |

In some embodiments, the reporter signals, analytes attached thereto, and reporter signal fusions are detected using mass spectrometry which allows sensitive distinctions between molecules based on their mass-to-charge ratios. The disclosed reporter signals can be used as general labels in myriad labeling and/or detection techniques. A set of isobaric reporter signals or reporter signal fusions can be used for multiplex labeling and/or detection of many analytes and/or detection of the expression of many genes, proteins, vectors, expression constructs, cells, cell lines, and organisms since the reporter signal fragments can be designed to have a large range of masses, with each mass individually distinguishable upon detection. Where the same analyte, gene, protein, vectors, expression construct, cell, cell line, or organism (or the same type of gene, protein, vector, expression construct, cell, cell line, or organism) is labeled with a set of isobaric reporter signals (by, for example, labeling the same gene, protein, vector, expression construct, cell, cell line, or organism in different samples), the set of reporter signals, reporter signal fusions, or labeled analytes that results from use of an isobaric set of reporter signals will also be isobaric. Fragmentation of the reporter signals will split the set of labeled proteins into individually detectable labeled proteins of characteristically different mass.

The method allows detection of proteins, peptides and protein fragments where detection provides some information on the sequence or other structure of the protein or peptide detected. For example, the mass or mass-to-charge ratio, the amino acid composition, or amino acid sequence can be determined. The set of proteins, peptides and/or protein fragments detected in a sample using particular reporter signals will produce characteristic sets of protein and peptide information. The method allows a complex sample of proteins to be cataloged quickly and easily in a reproducible manner. The disclosed method also should produce two “signals” for each protein, peptide, or peptide fragment in the sample: the original labeled protein and the altered form of the labeled protein. This can allow comparisons and validation of a set of detected proteins and peptides.

Reporter signal protein labeling allows profiling of proteins, de novo discovery of proteins, and cataloging of proteins. The method has advantageous properties which can be used as a detection and analysis system for protein analysis, proteome analysis, proteomic, protein expression profiling, de novo protein discovery, functional genomics, and protein detection.

C. Reporter Signal Calibration

In another form of the method, referred to as reporter signal calibration (RSC), a form of reporter signals referred to as reporter signal calibrators are mixed with analytes or analyte fragments, the reporter signal calibrators and the analytes or analyte fragments are altered, and the altered forms of the reporter signal calibrators and altered forms of the analytes or analyte fragments are detected. Reporter signal calibrators are useful as standards for assessing the amount of analytes present. That is, one can add a known amount of a reporter signal calibrator in order to assess the amount of analyte present comparing the amount of altered analyte or analyte fragment detected with the amount of altered reporter signal calibrator detected and calibrating these amounts with the known amount of reporter signal calibrator added (and thus the predicted amount of altered reporter signal calibrator).

The disclosed reporter signal calibration method generates, with high sensitivity, unique protein signatures related to the relative abundance of different proteins in tissue, microorganisms, or any other biological sample. The disclosed method allows one, for example, to define the status of a cell or tissue by identifying and measuring the relative concentrations of a small but highly informative subset of proteins. Such a measurement is known as a protein signature. Protein signatures are useful, for example, in the diagnosis, grading, and staging of cancer, in drug screening, and in toxicity testing.

In some embodiments, each analyte or analyte fragment can share one or more common properties with at least one reporter signal calibrator such that the reporter signal calibrators and analytes or analyte fragments having the common property can be distinguished and/or separated from other molecules lacking the common property.

In some embodiments, reporter signal calibrators and analytes and analyte fragments can be altered such that the altered form of an analyte or analyte fragment can be distinguished from the altered form of the reporter signal calibrator with which the analyte or analyte fragment shares a common property. In some embodiments, the altered forms of different reporter signal calibrators can be distinguished from each other. In some embodiments, the altered forms of different analytes or analyte fragments can be distinguished from each other.

In some embodiments of reporter signal calibration, the analyte or analyte fragment is not altered and so the altered reporter signal calibrators and the analytes or analyte fragments are detected. In this case, the analyte or analyte fragment can be distinguished from the altered form of the reporter signal calibrator with which the analyte or analyte fragment shares a common property.

In some embodiments the analyte or analyte fragment may be a reporter signal or a fragment of a reporter signal. In this case, the reporter signal calibrators serve as calibrators for the amount of reporter signal detected.

Reporter signal calibration is used, for example, in connection with proteins and peptides (as the analytes). This form of reporter signal calibration is referred to as reporter signal protein calibration. Reporter signal protein calibration is useful, for example, for producing protein signatures of protein samples. As used herein, a protein signature is the presence, absence, amount, or presence and amount of a set of proteins or protein surrogates.

In some embodiments of reporter signal protein calibration, the presence of labile, scissile, or cleavable bonds in the proteins to be detected can be exploited. Peptides, proteins, or protein fragments (collectively referred to, for convenience, as protein fragments in the remaining description) containing such bonds can be altered by fragmentation at the bond. In this way, reporter signal calibrators having a common property (such as mass-to-charge ratio) with the protein fragments can be used and the altered forms of the reporter signal calibrators and the altered (that is, fragmented) forms of the protein fragments can be detected and distinguished. In this regard, although the protein fragments share a common property with their matching reporter signal calibrators, the altered forms of the reporter signal calibrators and altered forms of protein fragments can be distinguished (because, for example, the altered forms have different properties, such as different mass-to-charge ratios).

As an example of reporter signal protein calibration, a protein sample of interest can be digested with a serine protease (e.g., trypsin). The digest generates a complex mixture of protein fragments. Among these protein fragments, there will exist a subset (approximately one protein fragment among every 400) that contains the dipeptide Asp-Pro. This dipeptide sequence is uniquely sensitive to fragmentation during mass spectrometry, and thus produces a high yield of ions in fragmentation mode. Since the human proteome consists of at least 2,000,000 distinct tryptic peptides, the number of protein fragments containing the Asp-Pro sequence is of the order of 5,000. Since some of these may exist as phosphopeptides or other modified forms, the number may be even higher. This number is sufficiently high to permit the selection of a subset (perhaps 50 to 100 or so) of fragmentable protein fragments that is suitable for generating a highly informative protein signature. Peptides that contain the Asp-Pro dipeptide sequence, peptides that contain amino acids that are modified by phosphorylation inside the cell, or peptides that contain an internal methionine are useful in reporter signal calibration. Alternatively, tryptic peptides terminating in arginine may be modified by reaction with acetylacetone (pentane-2,4-dione) to increase the frequency of fragment ions (Dikler et al., J Mass Spectrom 32:1337-49 (1997)). Selection of the subsets of protein fragments can be performed using bioinformatics in order to maximize the information content of the protein signatures.

For this form of reporter signal calibration, the protein digest can be mixed with a specially designed set of reporter signal calibrators, and then can be analyzed using tandem mass spectrometry. In the case of a tandem in space instrument (for example, Q-Tof™ from Micromass), using first quadrupole settings for single-ion filtering (defined by the molecular mass of each unique fragment, which can be obtained from sequence data), followed by a collision stage for ion fragmentation, and finally TOF spectrometry of the peptide fragments (generated by cleavage at fragile bonds, such as Asp-Pro, bonds involving a phosphorylated amino acid, or bonds adjacent to an oxidized amino-acid such as methionine sulfoxide, Smith et al., Free Radic Res. 26:103-11 (1997)) that arise from the original single-ion. In the second stage, signal to noise of the TOF measurement is much larger than in a conventional MS experiment. In general, one reporter signal calibrator can be used for each protein fragment in the sample that will be used to make up the protein signature (such protein fragments are referred to as signature protein fragments), and each is designed to fragment in an easily detectable pattern of masses, distinct from the fragment masses of the unfragmented signature protein fragments. The quadrupole filtering settings are then varied in sequence over a range of values (fifty, for example), corresponding to the masses of each of the protein fragments previously chosen to comprise the protein signature (that is, the signature protein fragments). At each filtered mass setting, there will be two types of signals detectable by TOF after fragmentation, one set derived from the tryptic peptide (that is, the original protein fragment), and another set corresponding to the reporter signal calibrator. The reporter signal calibrator permits one to calculate relative abundance for each of the signature protein fragments. These relative abundance ratios, determined for a given sample, constitute the protein signature. The detection limit of the tandem mass spectrometer in MS/MS mode, is remarkably good, perhaps of the order of 500 molecules of peptide. This level of detection is approximately 1,000 times better than that for MALDI-TOF mass spectrometry, and should permit the generation of protein signatures from single cells.

As can be seen, for this form of reporter signal calibration, the availability of the sequence of the entire human genome, as well as the genomes of many other organisms, can facilitate the identification of protein fragments that are unique in the context of all known proteins. That is, the sequence information can be used to identify peptides that will be generated in a protein signature and guide selection of reporter signal calibrators.

D. Rearranging Reporter Signals

Another embodiment of the disclosed method and compositions, referred to as rearranging reporter signals, enables one to detect the occurrence of specific gene rearrangement events, their protein products, and specific cell populations bearing those receptors. Rearranging reporter signals will also allow one to follow the progression or development of certain receptors and cells or populations of cells by monitoring the presence and/or absence of a reporter signal. Design considerations for rearranged reporter signals are analogous to those required for reporter signal fusions as described elsewhere herein.

Most embodiments of the disclosed method involve intact reporter signals that are associated with analytes in various ways. Rearranging reporter signals make use of processes, such as biological processes, to form reporter signals by specific rearrangement of the reporter signal pieces or rearrangement of nucleic acid segments encoding only portions of reporter signals. One form of rearranging reporter signals utilizes endogenous biological systems, such as the variable-diversity-joining (V-D-J) gene rearrangement machinery present in the mammalian immune system. In this system, short stretches of germline DNA (the V, D & J gene fragments) that are not contiguous, are brought together (recombined) prior to serving as a template for transcription. Gene rearrangement occurs in white blood cells such as T and B lymphocytes and is a key mechanism for generating diversity of T cell and B cell antigen receptors. Theoretically, billions of different receptors can be generated. This level of complexity makes it difficult to detect the presence of rare rearrangement events, or receptors. PCR based assays and flow cytometry approaches are now used to study receptor diversity. However, PCR approaches are laborious and do not provide any information on the status of expressed protein. Flow cytometry approaches have limited multiplexing capabilities due to emission spectra overlap of the fluorescent probes used.

If one desired to test for 50-100 T cell or B cell receptors, one would need to make use of a similar number of antibodies to those receptors, something that in practice is not done. Therefore, there is a real need for methods that would allow highly sensitive and specific detection of specific receptors in a highly complex pool of receptors. The ability to highly multiplex this approach would enable currently unattainable experimental approaches. The disclosed reporter signal technology allows large scale multiplexing of signals for detection.

As an example of rearranging reporter signals, transgenic mice can be generated in which nucleic acid sequences encoding reporter signals have been engineered into the mouse germline. Methods for doing this are well known in the art and include using standard molecular biology methods to engineer rearranging reporter signal into, for example, yeast or bacterial artificial chromosomes (YACs or BACs) and then using these constructs to generate transgenic mice.

As an example of the use of immunoglobulin rearrangement for rearranging reporter signals, part of a reporter signal could be encoded on the D region and another part of the reporter signal could be encoded on the J region. Upon a rearrangement event that joined the D and J regions encoding these “partial” reporter signals, a coding sequence for a “complete” reporter signal would be generated. Following transcription and translation, the reporter signal would be encoded within the protein product. The reporter signal could then be detected as described elsewhere herein. In the absence of a rearrangement event that joins the engineered D and J region, no reporter signal would be detected. By including sequences encoding parts of a variety of reporter signals with different D and J regions, a variety of different reporter signals can be generated by rearrangement, a different, and diagnostic, reporter signal for each of the different possible rearrangements. This system also could be extended to include, for example, reporter signals split among three or more gene regions (for example, V-D-J, V-D-D-J, etc) with the result that multiple rearrangement events would produce the reporter signal. In this mode, the combinations of rearrangements of the reporter signal parts can give rise to a large number of different reporter signals, each characterized by the specific reporter signal parts rearranged to form the reporter signal.

Transgenic mice carrying rearranging reporter signals would enable one to address questions that would otherwise be very difficult or impossible to address. For instance, one could dissect what specific T and B cell receptors (out of the thousands or millions possible) respond to specific stimuli or what cell types are present at certain stages of development.

E. Reporter Signal Fusions

Reporter signal fusions are reporter signal peptides joined with a protein or peptide of interest in a single amino acid segment (that is, a fusion protein). Such fusions of proteins and peptides of interest with reporter signal peptides can be expressed as a fusion protein or peptide from a nucleic acid molecule encoding the amino acid segment that constitutes the fusion. A reporter signal fusion nucleic acid molecule or reporter signal nucleic acid segment refers to a nucleic acid molecule or nucleic acid sequence, respectively, that encodes a reporter signal fusion. The reporter signal peptides, a form of reporter signal, allow for sensitive monitoring and detection of the proteins and peptides to which they are fused, and of expression of the genes, vectors, expression constructs, and nucleic acids that encode them. In particular, the reporter signal fusions allow sensitive and multiplex detection of expression of particular proteins and peptides of interest, and/or of the genes, vectors, and expression constructs encoding the proteins and peptides of interest. The disclosed reporter signal fusions can also be used for any purpose including as a source of reporter signals for other forms of the disclosed method and compositions.

As used herein “reporter signal fusion” refers to a protein, peptide, or fragment of a protein or peptide to which a reporter signal peptide is fused (that is, joined by peptide bond(s) in the same polypeptide chain) unless the context clearly indicates otherwise. The reporter signal peptide and the protein of interest involved in a reporter signal fusion need not be directly fused. That is, other amino acids, amino acid sequences, and/or peptide elements can intervene. For example, an epitope tag, if present, can be located between the protein of interest and the reporter signal peptide in a reporter signal fusion. The reporter signal peptide(s) can be fused to a protein in any arrangement, such as at the N-terminal end of the protein, at the C-terminal end of the protein, in or at domain junctions, or at any other appropriate location in the protein. In some forms of the method, it is desirable that the protein remain functional. In such cases, terminal fusions or inter-domain fusions are useful. Those of skill in the art of protein fusions generally know how to design fusions where the protein of interest remains functional. In other embodiments, it is not necessary that the protein remain functional in which case the reporter signal peptide and protein can have any desired structural organization.

Although reference is made above and elsewhere herein to detection of, and fusion with, a “protein” or “proteins,” the disclosed method and compositions encompass proteins, peptides, and fragments of proteins or peptides. Thus, reference to a protein herein is intended to refer to proteins, peptides, and fragments of proteins or peptides unless the context clearly indicates otherwise. As used herein “reporter signal fusion” refers to a protein, peptide, or fragment of a protein or peptide to which a reporter signal peptide is fused (that is, joined by peptide bond(s) in the same polypeptide chain) unless the context clearly indicates otherwise. The reporter signal fusion(s) can be fragmented, such as by protease digestion, prior to analysis. An expression sample to be analyzed can also be subjected to fractionation or separation to reduce the complexity of the samples. Fragmentation and fractionation can also be used together in the same assay. Such fragmentation and fractionation can simplify and extend the analysis of the expression.

The disclosed reporter signal fusions also are useful for creating cells, cell lines, and organisms that have particular protein(s), gene(s), vector(s), and/or expression sequence(s) labeled (that is, associated with or involved in) reporter signal fusions. For example, a set of nucleic acid constructs, each encoding a reporter signal fusion with a different reporter signal peptide, can be used to uniquely label a set of cells, cell lines, and/or organisms. Processing of a sample from any of the labeled sources can result in a unique altered form of the reporter signal peptide (or the amino acid segment or an amino acid subsegment) for each of the possible sources that can be distinguished from the other altered forms. Detection of a particular altered form identifies the source from which it came. As a more specific example, a nucleic acid construct encoding a reporter signal fusion can be introduced into a genetically modified plant line (for example, a Roundup resistant corn line) and the plant line can then be identified by detecting the reporter signal fusion. Non-limiting reporter signal peptides for use in reporter signal fusions used in or associated with different genes, proteins, vectors, constructs, cells, cell lines, or organisms would be those using differentially distributed mass. In particular, alternative amino acid sequences using the same amino acid composition is emplyed.

The disclosed method can also be used to assess the state and/or expression of particular pathways, regulatory cascades, and other suites of genes, proteins, vectors, and/or expressions sequences. The disclosed reporter signal fusions also can be used to “label” particular pathways, regulatory cascades, and other suites of genes, proteins, vectors, and/or expressions sequences. Such labeling can be within the same cell, cell line, or organism, or across a set of cells, cell lines, or organisms. For example, nucleic acid segments encoding reporter signal fusions can be associated with endogenous expression sequences of interest, endogenous genes of interest, exogenous expression sequences of interest, exogenous genes of interest, or a combination. The exogenous constructs then are introduced into the cells or organisms of interest. The association with endogenous expression sequences or genes can be accomplished, for example, by introducing a nucleic acid molecule (encoding the reporter signal fusion) for insertion at the site of the expression sequences or gene of interest, or by creating a vector or other nucleic acid construct (containing both the endogenous expression sequences or gene and a nucleic acid segment encoding the reporter signal fusion) in vitro and introducing the construct into the cells or organisms of interest. Many other uses and modes of use are possible, a number of which are described in the illustrations below. The disclosed reporter signal fusions can be used, for example, in any context and for any purpose that green fluorescent protein and green fluorescent protein fusions are used. However, the disclosed reporter signal proteins have more uses and are more useful than green fluorescent protein at least because of the ability to multiplex more highly the disclosed reporter signal fusions.

Nucleic acid sequences encoding reporter signal peptides can be engineered into particular exons of a gene. This would be the normal situation when the gene encoding the protein to be fused contains introns, although sequence encoding a reporter signal peptide can be split between different exons to be spliced together. Placement of nucleic acid sequences encoding reporter signal peptides into particular exons is useful for monitoring and analyzing alternative splicing of RNA. The appearance of a reporter signal peptide in the final protein indicates that the exon encoding the reporter signal peptide was spliced into the mRNA.

The reporter signal peptides can be used for sensitive detection of one or multiple proteins (that is, the proteins to which the reporter signal peptides are fused). In the method, proteins fused with reporter signal peptides are analyzed using the reporter signal peptides to distinguish the reporter signal fusions. Detection of the reporter signal peptides indicates the presence of the corresponding protein(s). The detected protein(s) can then be analyzed using known techniques. The reporter signal fusions provide a unique protein/label composition that can specifically identify the protein(s). This is accomplished through the use of the specialized reporter signal peptides as the labels.

The reporter signal fusions can be produced by expression from nucleic acid molecules encoding the fusions. Thus, the disclosed fusions generally can be designed by designing nucleic acid segments that encode amino acid segments where the amino acid segments comprise a reporter signal peptide and a protein or peptide of interest. A given nucleic acid molecule can comprise one or more nucleic acid segments. A given nucleic acid segment can encode one or more amino acid segments. A given amino acid segment can include one or more reporter signal peptides and one or more proteins or peptides of interest. The disclosed amino acid segments consist of a single, contiguous polypeptide chain. Thus, although multiple amino acid segments can be part of the same contiguous polypeptide chain, all of the components (that is, the reporter signal peptide(s) and protein(s) and peptide(s) of interest) of a given amino acid segment are part of the same contiguous polypeptide chain.

A protein to which the reporter signal peptide is fused to be identified by detection of one or more of the products of the reporter signal fusion following fragmentation, decomposition, reaction, derivatization, or other modification of the reporter signal peptide. Nucleic acid molecules and nucleic acid segments (i.e., part of a nucleic acid molecule) encoding reporter signal fusions can be used in sets where the reporter signal peptides in the reporter signal fusions encoded by a set of nucleic acid molecules can have one or more common properties that allow the reporter signal peptides to be separated or distinguished from molecules lacking the common property. Similarly, nucleic acid molecules encoding amino acid segments can be used in sets where the reporter signal peptides in the amino acid segments encoded by a set of nucleic acid molecules can have one or more common properties that allow the reporter signal peptides to be separated or distinguished from molecules lacking the common property. Nucleic acid molecules encoding amino acid segments can be used in sets where the amino acid segments encoded by a set of nucleic acid molecules can have one or more common properties that allow the amino acid segments to be separated or distinguished from molecules lacking the common property. Other relationships between members of the sets of nucleic acid molecules, nucleic acid segments, amino acid segments, reporter signal peptides, and proteins of interest are contemplated.

Reporter signal fusions can include other components besides a protein of interest and a reporter signal peptide. For example, reporter signal fusions can include epitope tags or flag peptides (see, for example, Brizzard et al. (1994) Immunoaffinity purification of FLAG epitope-tagged bacterial alkaline phosphatase using a novel monoclonal antibody and peptide elution. Biotechniques 16:730-735). Epitope tags and flag peptides can serve as tags by which reporter signal fusions can be manipulated. The use of epitope tags and flag peptides generally is known and can be adapted for use in the disclosed reporter signal fusions.

Cells, cell lines, organisms, and expression of genes and proteins can be detected using the disclosed reporter signal fusions in a variety of ways.

A non-limiting form of the disclosed method involves correlated detection of the reporter signal peptides both before and after fragmentation of the reporter signal peptide. This allows genes, proteins, vectors, and expression constructs “labeled” with a reporter signal peptide to be detected and identified via the change in the reporter signal fusion and/or reporter signal peptide. That is, the nature of the reporter signal fusion or reporter signal peptide detected (non-fragmented versus fragmented) identifies the gene, protein, vector, or nucleic acid construct from which it was derived. Where the reporter signal fusions and reporter signal peptides are detected by mass-to-charge ratio, the change in mass-to-charge ratio between fragmented and non-fragmented samples provides the basis for comparison. Such mass-to-charge ratio detection is accomplished, for example, with mass spectrometry.

As an example, a fusion between a protein of interest and a reporter signal peptide can be expressed. The reporter signal fusion can be subjected to tryptic digest followed by mass spectrometry of the resulting materials. (Note that this example can also apply to an analyte in a sample labeled with a reporter signal) A peak corresponding to the tryptic fragment (or corresponding to the analyte/reporter label) containing the reporter signal peptide will be detected. Fragmentation of the reporter signal peptide in the mass spectrometer (e.g., in a collision cell) would result in a shift in the peak corresponding to the loss of a portion of the attached reporter signal peptide, the appearance of a peak corresponding to the lost fragment, or a combination of both events. Significantly, the shift observed will depend on which reporter signal peptide is fused to the protein since different reporter signal peptides will, by design, produce fragments with different mass-to-charge ratios. The combination event of detection of the parent mass-to-charge (with no collision gas) and the mass-to-charge corresponding to the loss of the fragment from the reporter signal peptide (with collision gas) indicates a reporter signal fusion (thus indicating expression of the reporter signal fusion and of the gene, vector, or construct encoding it), or indicates the labeled analyte. The identity of the analyte can be determined by standard mass spectrometry techniques, such as compositional analysis.

A powerful form of the disclosed method is use of reporter signals (e.g., attached to analytes) and/or reporter signal fusions to assay multiple samples (for example, time series assays or other comparative analyses). Knowledge of the temporal response of a biological system following perturbation is a very powerful process in the pursuit of understanding the system. To follow the temporal response a sample of the system is obtained (for example, cells from a cell culture, mice initially synchronized and sacrificed) at determined times following the perturbation. Knowledge of spatial protein profiles (for example, relative position within a tissue section) is a very powerful process in the pursuit of understanding the biological system.

Nucleic acid sequences and segments encoding reporter signals and/or reporter signal fusions can be expressed in any suitable manner. For example, the disclosed nucleic acid sequences and nucleic acid segments can be expressed in vitro, in cells, and/or in cells in organism. Many techniques and systems for expression of nucleic acid sequences and proteins are known and can be used with the disclosed reporter signal fusions. For example, many expression sequences, vector systems, transformation and transfection techniques, and transgenic organism production methods are known and can be used with the disclosed reporter signal peptide method and compositions. Systems are known for integration of nucleic acid constructs into chromosomes of cells and organisms (see, for example, Groth et al. (2000) A phage integrase directs efficient site-specific integration in human cells. Proc Natl Acad Sci USA 97:5995-6000; Hong et al. (2001) Development of two bacterial artificial chromosome shuttle vectors for a recombination-based cloning and regulated expression of large genes in mammalian cells. Analytical Biochemistry 291:142-148) which can be used with the disclosed nucleic acid molecules and segments encoding reporter signal fusions or to form nucleic acid segment encoding reporter signal fusions.

For example, kits for the in vitro transcription/translation of DNA constructs containing promoters and nucleic acid sequence to be transcribed and translated are known (for example, PROTEINscript-PRO™ from Ambion, Inc. Austin Tex.; Wilkinson (1999) “Cell-Free And Happy: In Vitro Translation And Transcription/Translation Systems”, The Scientist 13[13]:15, Jun. 21, 1999). Such constructs can be used in the genomic DNA of an organism, in a plasmid or other vector that may be transfected into an organism, or in in vitro systems. For example, constructs containing a promoter sequence and a nucleic acid sequence that, following transcription and translation, results in production of green fluorescence protein or luciferase as a reporter/marker in in vivo systems are known (for example, Sawin and Nurse, “Identification of fission yeast nuclear markers using random polypeptide fusions with green fluorescent protein.” Proc Natl Acad Sci USA 93(26): 15146-51 (1996); Chatterjee et al., “In vivo analysis of nuclear protein traffic in mammalian cells.” Exp Cell Res 236(1):346-50 (1997); Patterson et al., “Quantitative imaging of TATA-binding protein in living yeast cells.” Yeast 14(9):813-25 (1998); Dhandayuthapani et al., “Green fluorescent protein as a marker for gene expression and cell biology of mycobacterial interactions with macrophages.” Mol Microbiol 17(5):901-12 (1995); Kremer et al., “Green fluorescent protein as a new expression marker in mycobacteria.” Mol Microbiol 17(5):913-22 (1995); Reiländer et al., “Functional expression of the Aequorea victoria green fluorescent protein in insect cells using the baculovirus expression system.” Biochem Biophys Res Commun 219(1): 14-20 (1996); Mankertz et al., “Expression from the human occludin promoter is affected by tumor necrosis factor alpha and interferon gamma” J Cell Sci, 113:2085-90 (2000); White et al., “Real-time analysis of the transcriptional regulation of HIV and hCMV promoters in single mammalian cells” J Cell Sci, 108:441-55 (1995)). Green fluorescence protein, or variants, have been shown to be stably incorporated and not interfere with the organism—generally GFP is larger relative to the disclosed reporter signal peptides (GFP from Aequorea Victoria is 238 amino acids in size; NCBI GI:606384), and thus the generally smaller reporter signal peptides are less likely to disrupt an expression system to which they are added.

Techniques are known for modifying promoter regions such that the endogenous promoter is replaced with a promoter-reporter construct, for example, where the reporter is green fluorescent protein (Patterson et al., “Quantitative imaging of TATA-binding protein in living yeast cells.” Yeast 14(9): 813-25 (1998)) or luciferase. Transcription factor concentrations are followed by monitoring the GFP or luciferase. These techniques can be used with the disclosed reporter signal fusions and reporter signal fusion constructs. Techniques are also known for targeted knock-in of nucleic acid sequences into a gene of interest, typically under control of the endogenous promoter. Such techniques, which can be used with the disclosed method and compositions, have been used to introduce reporter/markers of the transcription and translation of the gene where the nucleic acid was inserted. The same techniques can be used to place the disclosed reporter signal fusions under control of endogenous expression sequences. Alternately, non-targeted knock-ins (techniques for which are also known; Hobbs et al. “Development of a bicistronic vector driven by the human polypeptide chain elongation factor 1 alpha promoter for creation of stable mammalian cell lines that express very high levels of recombinant proteins” Biochem Biophys Res Commun, 252:368-72 (1998); Kershnar et al., “Immunoaffinity purification and functional characterization of human transcription factor 11H and RNA polymerase II from clonal cell lines that conditionally express epitope-tagged subunits of the multiprotein complexes” J Biol Chem, 273:34444-53 (1998); Wu and Chiang, “Establishment of stable cell lines expressing potentially toxic proteins by tetracycline-regulated and epitope-tagging methods” Biotechniques 21:718-22, 724-5 (1996)) can be used to follow the level or activity of transcription factors—reporter signal peptide fusions associated with the inserted nucleic acid code can directly indicate the transcription/translation activity.

The disclosed reporter signal fusions also can be used in the detection and analysis of protein interactions with other proteins and molecules. For example, interaction traps for protein-protein interactions include the well known yeast two-hybrid (Fields and Song, “A novel genetic system to detect protein-protein interactions” Nature 340:245-6 (1989); Uetz et al., “A comprehensive analysis of protein-protein interactions in Saccharomyces cerevisiae” Nature 403:623-7 (2000)) and related systems (Ausubel et al., Current Protocols in Molecular Biology, John Wiley & Sons, Inc., 2001; Van Criekinge and Beyaert, “Yeast two-hybrid: state of the art” Biological Procedures Online, 2(1), 1999). Incorporation of nucleic acid sequence encoding a peptide reporter signal can be introduced into these systems, for example at a terminus of the ordinarily used LacZ selection region (LacZ selection is described in, for example, Sambrook et al., Molecular Cloning: A Laboratory Manual, second edition, 1989, Cold Spring Harbor Laboratory Press, New York). A set of such incorporated sequences (for example, in a set of such plasmids, where each plasmid has a reporter signal coding sequence and the LacZ functionality), allows the unambiguous detection of many interactions simultaneously rather (as many different interactions as reporter signals used).

In another mode of reporter signal fusions, a nucleic acid sequence encoding a reporter signal could be added to sequence encoding the constant (C) region of T cell and B cell receptors. The reporter signal would appear in T or B cell receptors when that C region is spliced to a J region following transcription.

In another mode of reporter signal fusions, referred to as reporter signal presentation, the presentation of specific antigenic peptides by major histocompatibility (MHC) and non-major histocompatibility molecules can be detected and analyzed. It is well known that protein antigens are processed by antigen presenting cells and that small peptides, typically 8-12 amino acids are presented by Class I and Class II MHC molecules for recognition by T cells. The study of specific T cell/peptide-MHC complexes is technically challenging due various labeling requirements (either radioactive or fluorescence) and the common reliance on antibody reagents that recognize specific receptors and/or peptide-MHC complexes.

There is a need to be able to further expand our knowledge of antigen processing and antigen presentation. Reporter signals that have been engineered into specific protein antigens could provide novel insight into this process and enable new experimental approaches. For instance, consider two viral or bacterial proteins, protein A and protein B, that differ by only a few amino acids. It would be useful to know if they are processed and presented to immune cells (for example, T cells) with the same efficiency. By engineering reporter signals into protein A and engineered protein B to antigen presenting cells, one could test for the presence of the different reporter signals presented on and thus determine if the proteins are efficiently processed and presented. The presence of reporter signal A (present in protein A) but not reporter signal B (present in protein B), indicates that protein A is processed and that protein B is not. The lack of antigen processing of protein B may then be an explanation of why a virus or bacteria escapes immune surveillance by the immune system. Antigenic peptides are characterized by conserved anchor residues near both the amino and carboxy ends, with more heterogeneity tolerated in the middle. This middle heterogeneity is thus a useful site for addition of a reporter signal peptide.

A given reporter signal fusion can include one or more reporter signal peptides and one or more proteins or peptides of interest. In addition, reporter signal fusions can include one or more amino acids, amino acid sequences, and/or peptide elements. The disclosed reporter signal fusions comprise a single, contiguous polypeptide chain. Thus, although multiple amino acid segments can be part of the same contiguous polypeptide chain, all of the components (that is, the reporter signal peptide(s) and protein(s) and peptide(s) of interest) of a given amino acid segment are part of the same contiguous polypeptide chain.

Reporter signal fusions can be produced by expression from nucleic acid molecules encoding the fusions. Thus, the disclosed fusions generally can be designed by designing nucleic acid segments that encode amino acid segments where the amino acid segments comprise a reporter signal peptide and a protein or peptide of interest. A given nucleic acid molecule can comprise one or more nucleic acid segments. A given nucleic acid segment can encode one or more amino acid segments. A given amino acid segment can include one or more reporter signal peptides and one or more proteins or peptides of interest. The disclosed amino acid segments consist of a single, contiguous polypeptide chain. Thus, although multiple amino acid segments can be part of the same contiguous polypeptide chain, all of the components (that is, the reporter signal peptide(s) and protein(s) and peptide(s) of interest) of a given amino acid segment are part of the same contiguous polypeptide chain.

As used herein, an expression sample is a sample that contains, or might contain, one or more reporter signal fusions expressed from a nucleic acid molecule. An expression sample to be analyzed can be subjected to fractionation or separation to reduce the complexity of the samples. Fragmentation and fractionation can also be used together in the same assay. Such fragmentation and fractionation can simplify and extend the analysis of the expression.

Reporter signal fusions can include other components besides a protein of interest and a reporter signal peptide. For example, reporter signal fusions can include epitope tags or flag peptides (see, for example, Groth et al. (2000) A phage integrase directs efficient site-specific integration in human cells. Proc Natl Acad Sci USA 97:5995-6000). Epitope tags and flag peptides can serve as tags by which reporter signal fusions can be separated, distinguished, associated, and/or bound. The use of epitope tags and flag peptides generally is known and can be adapted for use in the disclosed reporter signal fusions.

Alteration of reporter signals peptides in reporter signal fusions can produce a variety of altered compositions. Any or all of these altered forms can be detected. For example, the altered form of the reporter signal peptide can be detected, the altered form of the amino acid segment (which contains the reporter signal peptide) can be detected, the altered form of a subsegment of the amino acid segment can be detected, or a combination of these can be detected. Where the reporter signal peptide is altered by fragmentation, the result generally will be a fragment of the reporter signal peptide and an altered form of the amino acid segment containing the protein or peptide of interest and a portion of the reporter signal peptide (that is, the portion not in the reporter signal peptide fragment).

The protein or peptide of interest also can be fragmented. The result would be a subsegment of the amino acid segment. The amino acid subsegment would contain the reporter signal peptide and a portion of the protein or peptide of interest. When the reporter signal peptide in an amino acid subsegment is altered (which can occur before, during, or after fragmentation of the amino acid segment), the result is an altered form of the amino acid subsegment (and an altered form of the reporter signal peptide). This altered form of amino acid subsegment can be detected. Where the reporter signal peptide is altered by fragmentation, the result generally will be a fragment of the reporter signal peptide and an altered form of (that is, fragment of) the amino acid subsegment. In this case, the altered form of the amino acid subsegment, which is also referred to herein as a reporter signal fusion fragment, will contain a portion of the protein or peptide of interest and a portion of the reporter signal peptide (that is, the portion not in the reporter signal peptide fragment).

Sets of reporter signals, reporter signal fusions (also referred to as amino acid segments), reporter signal fusion fragments (also referred to as subsegments of the reporter signal fusions or amino acid subsegments), reporter signal peptides, nucleic acid segments encoding reporter signal fusions, or nucleic acid molecules comprising nucleic acid segments encoding reporter signal fusions can have any number of reporter signals, reporter signal fusions, reporter signal fusion fragments, reporter signal peptides, nucleic acid segments encoding reporter signal fusions, or nucleic acid molecules comprising nucleic acid segments encoding reporter signal fusions. For example, sets of reporter signals, reporter signal fusions, reporter signal fusion fragments, reporter signal peptides, nucleic acid segments encoding reporter signal fusions, or nucleic acid molecules comprising nucleic acid segments encoding reporter signal fusions can have one, two or more, three or more, four or more, five or more, six or more, seven or more, eight or more, nine or more, ten or more, twenty or more, thirty or more, forty or more, fifty or more, sixty or more, seventy or more, eighty or more, ninety or more, one hundred or more, two hundred or more, three hundred or more, four hundred or more, or five hundred or more different reporter signals, reporter signal fusions, reporter signal fusion fragments, reporter signal peptides, nucleic acid segments encoding reporter signal fusions, or nucleic acid molecules comprising nucleic acid segments encoding reporter signal fusions. Although specific numbers of reporter signals, reporter signal fusions, reporter signal fusion fragments, reporter signal peptides, nucleic acid segments encoding reporter signal fusions, and nucleic acid molecules comprising nucleic acid segments encoding reporter signal fusions, and specific endpoints for ranges of the number of reporter signals, reporter signal fusions, reporter signal fusion fragments, reporter signal peptides, nucleic acid segments encoding reporter signal fusions, and nucleic acid molecules comprising nucleic acid segments encoding reporter signal fusions, are recited, each and every specific number of reporter signals, reporter signal fusions, reporter signal fusion fragments, reporter signal peptides, nucleic acid segments encoding reporter signal fusions, and nucleic acid molecules comprising nucleic acid segments encoding reporter signal fusions, and each and every specific endpoint of ranges of numbers of reporter signals, reporter signal fusions, reporter signal fusion fragments, reporter signal peptides, nucleic acid segments encoding reporter signals, reporter signal fusions, and nucleic acid molecules comprising nucleic acid segments encoding reporter signals, reporter signal fusions, are specifically contemplated, although not explicitly listed, and each and every specific number of reporter signal fusions, reporter signal fusion fragments, reporter signal peptides, nucleic acid segments encoding reporter signal fusions, and nucleic acid molecules comprising nucleic acid segments encoding reporter signals, reporter signal fusions, and each and every specific endpoint of ranges of numbers of reporter signal fusions, reporter signal fusion fragments, reporter signal peptides, nucleic acid segments encoding reporter signal fusions, and nucleic acid molecules comprising nucleic acid segments encoding reporter signals, reporter signal fusions, are hereby specifically described.

Reporter signal fusions can be used to monitor and analyze alternative RNA splicing. A central problem in translating the information in the genome to protein expression is an understanding of mRNA alternative processing, and the generation of protein isoforms via alternative exon utilization (Black, “Protein diversity from alternative splicing: a challenge for bioinformatics and post-genome biology” Cell 103:367-70 (2000)). Many examples of the use of alternative pre-mRNA splicing to generate protein isoform diversity exist, such as in the control of erythroid differentiation (see, for example, Hou and Conboy, “Regulation of alternative pre-mRNA splicing during erythroid differentiation” Curr Opin Hematol 8:74-9 (2001)). Often the detection of complex, alternatively spliced protein isoforms is a difficult task, since exons may be as small as 6 amino acids in protein of over 2000 amino acids (see, for example, Cianci et al., “Brain and muscle express a unique alternative transcript of all spectrin” Biochem 38:15721-15730 (1999)).

Exon utilization and processing information can be obtained by insertion of a nucleic acid sequence encoding a reporter signal into the exon sequence of interest (thus forming a nucleic acid segment that encodes a reporter signal fusion). The insertions can be made, for example, into genomic DNA, appropriate mini-gene constructs, or non-endogenous pre-mRNA introduced into the cell. Use of a set of reporter signals allows the multiplexed readout of all exons of a translated protein at one time. The use of mini-gene constructs or constructs incorporating short exogenous open-reading frame DNA sequences into exons, and the incorporation of foreign DNA in association with functional intron splice elements are developed technologies that can be used for incorporation of reporter signals (see, for example, Gee et al., “Alternative splicing of protein 4.1R exon 16: ordered excision of flanking introns ensures proper splice site choice” Blood 95:692-9 (2000); Kikumori et al., “Promiscuity of pre-mRNA spliceosome-mediated trans splicing: a problem for gene therapy?” Hum Gene Ther 12:1429-41 (2001); Malik et al., “Effects of a second intron on recombinant MFG retroviral vector” Arch Virol 146:601-9 (2001); Virts and Raschke, “The role of intron sequences in high level expression from CD45 cDNA constructs” J Biol Chem 276:19913-20 (2001)). Detection of the reporter signals, the amounts of the reporter signals, and the knowledge of which reporter signal correlates with which exon, provides information about exon usage and alternative splicing.

Thus, reporter signal fusions allow sensitive and multiplex detection of expression of particular proteins and peptides of interest, and/or of the genes, vectors, and expression constructs encoding the proteins and peptides of interest. The disclosed reporter signal fusions can also be used for any purpose including as a source of reporter signals for other forms of the disclosed method and compositions.

F. Reporter Signal/Analyte Conjugates

Compositions where reporter signals are associated with, incorporated into, or otherwise linked to the analytes are referred to as reporter signal/analyte conjugates. Such conjugates include reporter signals associated with analytes, such as a reporter signal probe hybridized to a nucleic acid sequence; reporter signals covalently coupled to analytes, such as reporter signals linked to proteins via a linking group; and reporter signals incorporated into analytes, such as fusions between a protein of interest and a peptide reporter signal.

Reporter signal/analyte conjugates can be altered, generally through alteration of the reporter signal portion of the conjugate, such that the altered forms of different reporter signals, altered forms of different reporter signal/analyte conjugates, or both, can be distinguished from each other. Where the reporter signal or reporter signal/analyte conjugate is altered by fragmentation, any, some, or all of the fragments can be distinguished from each other, depending on the embodiment. For example, where reporter signal/analyte conjugates are fragmented into two parts (with the break point in the reporter signal portion), either the reporter signal fragment, the reporter signal/analyte fragment, or both can be distinguished.

Sets of reporter signal/analyte conjugates can be used where two or more of the reporter signal/analyte conjugates in a set have one or more common properties that allow the reporter signal/analyte conjugates having the common property to be distinguished and/or separated from other molecules lacking the common property. In still other embodiments, analytes can be fragmented (prior to or following conjugation) to produce reporter signal/analyte fragment conjugates (which can be referred to as fragment conjugates). In such cases, sets of fragment conjugates can be used where two or more of the fragment conjugates in a set have one or more common properties that allow the fragment conjugates having the common property to be distinguished and/or separated from other molecules lacking the common property. It should be understood that fragmented analytes can be considered analytes in their own right. In this light, reference to fragmented analytes is made for convenience and clarity in describing certain embodiments and to allow reference to both the base analyte and the fragmented analyte.

As indicated above, reporter signals conjugated with analytes can be altered while in the conjugate and distinguished. Conjugated reporter signals can also be dissociated or separated, in whole or in part, from the conjugated analytes prior to their alteration. Where the reporter signals are dissociated (in whole or in part) from the analytes, the method can be performed such that the fact of association between the analyte and reporter signal is part of the information obtained when the reporter signal is detected. In other words, the fact that the reporter signal may be dissociated from the analyte for detection does not obscure the information that the detected reporter signal was associated with the analyte.

As used herein, reporter signal conjugate refers both to reporter signal/analyte conjugates and to other components of the disclosed method such as reporter molecules.

As with reporter signals generally, reporter signal fusions, reporter signal/analyte conjugates and reporter signal/analyte fragment conjugates can be used in sets where the reporter signal fusions, reporter signal/analyte conjugates or fragment conjugates in a set can have one or more common properties that allow the reporter signal fusions, reporter signal/analyte conjugates or fragment conjugates to be separated or distinguished from molecules lacking the common property. In the case of reporter signal fusions, amino acid segments and amino acid subsegments can be used in sets where the amino acid segments and amino acid subsegments in a set can have one or more common properties that allow the amino acid segments and amino acid subsegments, respectively, to be separated or distinguished from molecules lacking the common property. In general, the component(s) of the reporter signal fusions having common properties can depend on the component(s) to be detected and/or the mode of the method being used.

A variety of different properties can be used as the common physical property used to separate the reporter signal, reporter signal fusions, reporter signal/analyte conjugates or fragment conjugates from other molecules lacking the common property. For example, physical properties useful as common properties include mass-to-charge ratio, mass, charge, isoelectric point, hydrophobicity, chromatography characteristics, and density. In some embodiments, the physical property shared by the reporter signal, reporter signal fusions, reporter signal/analyte conjugates or fragment conjugates in a set (and used to distinguish or separate the reporter signal, reporter signal fusions, reporter signal/analyte conjugates or fragment conjugates from other molecules) is an overall property of the reporter signal, reporter signal fusions, reporter signal/analyte conjugates or fragment conjugates (for example, overall mass, overall charge, isoelectric point, overall hydrophobicity, etc.) rather than the mere presence of a feature or moiety (for example, an affinity tag, such as biotin). Such properties are referred to herein as “overall” properties (and thus, reporter signal, reporter signal fusions, reporter signal/analyte conjugates or fragment conjugates in a set would be referred to as sharing a “common overall property”). It should be understood that reporter signal, reporter signal fusions, reporter signal/analyte conjugates or fragment conjugates can have features and moieties, such as affinity tags, and that such features and moieties can contribute to the common overall property (by contributing mass, for example). However, such limited and isolated features and moieties would not serve as the sole basis of the common overall property.

In some embodiments, the common property of reporter signal fusions, reporter signal fusion fragments, or reporter signal peptides is not an affinity tag. Nevertheless, even in such a case, reporter signal fusions, reporter signal fusion fragments, or reporter signal peptides that otherwise have a common property may also include an affinity tag—and in fact may all share the same affinity tag—so long as another common property is present that can be (and, in some embodiments of the disclosed method, is) used to separate reporter signal fusions, reporter signal fusion fragments, or reporter signal peptides sharing the common property from other molecules lacking the common property. With this in mind, if chromatography or other separation techniques are used to separate reporter signal fusions, reporter signal fusion fragments, or reporter signal peptides based on the common property, then in these embodiments the affinity tag may be based on an overall physical property of the reporter signal fusions, reporter signal fusion fragments, or reporter signal peptides and not on the presence of, for example, a feature or moiety such as an affinity tag. As used herein, a common property is a property shared by a set of components (such as reporter signal fusions, reporter signal fusion fragments, or reporter signal peptides). That is, the components have the property “in common.” It should be understood that reporter signal fusions, reporter signal fusion fragments, or reporter signal peptides in a set may have numerous properties in common. However, as used herein, the common properties of reporter signal fusions, reporter signal fusion fragments, or reporter signal peptides referred to are only those used in the disclosed method to distinguish and/or separate the reporter signal fusions, reporter signal fusion fragments, or reporter signal peptides sharing the common property from molecules that lack the common property.

G. Lipid Reporter Signals

The disclosed method and compositions also can be used to monitor lipid composition, distribution, and processing. Lipids are hydrophobic biomolecules that have high solubility in organic solvents. They have a variety of biological roles that make them valuable targets for monitoring. As a nutritional source, lipids (together with carbohydrates) constitute an important source of cellular energy and metabolic intermediates needed for cell signaling and other processes. Lipids processed for energy conversion typically pass through a variety of enzymatic pathways, generating many intermediates. A summary of these cycles is available in most modern biochemistry texts (see, for example, Stryer, 1995). Monitoring the processing of acyl chain intermediates as they are metabolized is an important tool in lipid and cell biological research, as well as for the clinical detection of biochemical diseases such as medium-chain acyl-CoA dehydrogenase deficiencies (see, for example, Zschocke et al., “Molecular and functional characterization of mild MCAD deficiency.”, Hum Genet. 108:404-8 (2001)). Incorporating reporter signals into, or associating reporter signals with, lipids can improve methods of detecting lipids (such as Andresen et al., “Medium-chain acyl-CoA dehydrogenase (MCAD) mutations identified by MS/MS-based prospective screening of newborns differ from those observed in patients with clinical symptoms: identification and characterization of a new, prevalent mutation that results in mild MCAD deficiency” Am J Hum Genet. 68:1408-18. (2001)) by allowing, for example, more rapid and multiplex detection of processed acyl chain intermediates.

In another role, lipids function as the most fundamental and defining component of all biological membranes. The three major types of membrane lipids are phospholipids, glycolipids, and cholesterol. The most abundant of these are the phospholipids, derived either from glycerol or sphingosine. Those based on glycerol typically contain two esterified long-chain fatty acids (14 to 24 carbons) and a phosphorylated alcohol or sugar. Phospholipids based on sphingosine contain a single fatty acid. Collectively these lipids contribute to the structure and fluidity of biological membranes. Cyclic changes in their processing, particularly of acidic glycophosolipids such as phosphatidyl inositol 4,5 phosphate, also regulate a wide variety of cellular processes (see, for example, Cantrell, “Phosphoinositide 3-kinase signaling pathways” J Cell Sci 114:1439-45 (2001); Payrastre et al., “Phosphoinositides: key players in cell signaling, in time and space” Cell Signal 13:377-87 (2001)). Thus, by incorporating reporter signals into, or associating reporter signals with, the acyl chains of such molecules, the subsequent incorporation of such reporter molecules into either in vitro assays such as those used for enzyme determinations or in vivo assays, allows one to rapidly follow the segregation of these lipids into distinct cellular compartments (for example, golgi versus plasma membrane (see, for example, Godi et al., “ARF mediates recruitment of PtdIns-4-OH kinase-beta and stimulates synthesis of PtdIns(4,5)P2 on the Golgi complex” Nat Cell Biol 1:280-7 (1999)), and their processing via metabolic and signaling pathways such as those cited above.

It is known that exogenous lipid labels can be incorporated readily into biological systems, and the disclosed reporter signals also can be incorporated into such systems. For example, spin-labeled acyl fatty acids and phospholipids have been incorporated into the membranes of phospholipid vesicles and cells (see, for example, Kornberg and McConnell, “Inside-outside transitions of phospholipids in vesicle membranes” Biochemistry 10:1111-20 (1971); Kornberg and McConnell, “Lateral diffusion of phospholipids in a vesicle membrane” Proc Natl Acad Sci USA 68:2564-8 (1971); Arora et al., “Selectivity of lipid-protein interactions with trypsinized Na, K-ATPase studied by spin-label EPR” Biochim Biophys Acta 1371:163-7 (1998); Alonso et al., “Lipid chain dynamics in stratum corneum studied by spin label electron paramagnetic resonance” Chem Phys Lipids 104:101-11 (2000)).

Triglycerides, or the acyl chain of sphinoglipids or glycolipids, and cholesterol, may be synthesized to include a reporter signal. An example of such a reporter signal would be a lipid made from an aliphatic chain with a carboxylic acid with a photocleavable bond. Examples of photocleavable bonds are described by Glatthar and Geise, Org. Lett, 2:2315-2317 (2000); Guillier et al., Chem. Rev. 100:2091-2157 (2000); Wierenga, U.S. Pat. No. 4,086,254; and elsewhere here. A set of reporter signals may be prepared by locating the cleavable bond at different locations within an aliphatic chain (thus resulting in fragments of different mass when the bond is cleaved). The aliphatic chain with a photocleavable bond constitutes the reporter signal. Such synthetic reporter molecules can be incorporated into synthetic triglycerides by, for example, a dehydration reaction. Once formed, a set of these synthetic triglycerides can be introduced into biological systems of interest, such as those described above. Reporter signals can be recovered from the biological system of interest for detection and quantitation by, for example, extraction of the lipid into chloroform and release of reporter signals from the trigyceride using a lipase or hydrolysis reaction.

H. Separation and Capture of Reporter Signals

Binding of a specific binding molecule to reporter signal(s) can take various forms, and/or be characterized in various ways. For example, the specific binding molecule can bind reporter signals in a set of reporter signals, reporter signals in two or more sets of reporter signals, reporter signal calibrator(s), reporter signal calibrators in a set of reporter signal calibrators, target protein fragments, reporter signal peptides, reporter signals peptides in a set of reporter signal peptides, amino acid segments (such as amino acid segments that comprise a reporter signal peptide), amino acid subsegments (such as amino acid segments that comprise a reporter signal peptide), reporter signal/analyte conjugates. The specific binding molecule specific for a reporter signal can be chosen to be capable of binding the altered form of the reporter signal (and/or of the molecule to which the reporter signal is attached—for example, the altered form of a labeled protein).

In some forms of the disclosed method, reporter signals can be detected via binding to a specific binding molecule. This can be done, for example, prior to separation and detection of altered forms of the reporter signals. For example, reporter signals attached or bound to analytes can be separated by chromatography and/or electrophoresis and detected by binding to specific binding molecules and detection of the specific binding molecules (such as via a label on or associated with the specific binding molecule. For example, reporter signals can be detected using Western blotting. Following such detection, reporter signals can be collected (by elution or extraction, for example) and processed by, for example, separation based on a common property, alteration of the reporter signals, and detection of the altered forms of the reporter signals. Although for convenience the above description refers to reporter signals, it should be understood that various other components described elsewhere herein that include or comprise a reporter signal (such as labeled proteins) can be used in the same manner. Thus, for example, labeled proteins can be separated by chromatography and/or electrophoresis and detected by binding to specific binding molecules and detection of the specific binding molecules (such as via a label on or associated with the specific binding molecule. Following such detection, the labeled proteins can be collected (by elution or extraction, for example) and the labeled proteins processed by, for example, separation based on a common property, alteration of the labeled proteins, and detection of the altered form of the labeled proteins.

I. Materials

a. Reporter Signals

Reporter signals are molecules that can be, for example, fragmented, decomposed, reacted, derivatized, or otherwise modified or altered for detection. Detection of the modified reporter signals is accomplished, for example, with mass spectrometry. The disclosed reporter signals may be used in sets where members of a set have the same mass-to-charge ratio (m/z). This facilitates sensitive filtering or separation of reporter signals from other molecules based on mass-to-charge ratio. Reporter signals can have any structure that allows modification of the reporter signal and identification of the different modified reporter signals. Reporter signals are, for example, composed such that at least one preferential bond rupture can be induced in the molecule. A set of reporter signals having nominally the same molecular mass and arbitrarily chosen internal fragmentation points may be constructed such that upon fragmentation each member of the set will yield unique correlated daughter fragments. For convenience, reporter signals that are fragmented, decomposed, reacted, derivatized, or otherwise modified for detection are referred to as fragmented reporter signals.

Non-limiting reporter signals are made up of chains of subunits such as peptides, oligonucleotides, peptide nucleic acids, oligomers, carbohydrates, polymers, and other natural and synthetic polymers and any combination of these. Non-limiting chains are peptides, and are referred to herein as reporter signal peptides. Chains of subunits and subunits have a relationship similar to that of a polymers and mers. The mers are connected together to form a polymer. Likewise, subunits are connected together to form chains of subunits. Non-limiting reporter signals are made up of chains of similar or related subunits. These are termed homochains or homopolymers. For example, nucleic acids are made up of phosphonucleosides and peptides are made up of amino acids.

Reporter signals can also be made up of heterochains or heteropolymers. A heterochain is a chain or a polymer where the subunits making up the chain are different types or the mers making up the polymer are different types. For example, a heterochain could be guanosine-alanine, which is made up of one nucleoside subunit and one amino acid subunit. It is understood that any combination of types of subunits can be used within the disclosed compositions, sets, and methods. Any molecule having the required properties can be used as a reporter signal. In some embodiments, reporter signals can be fragmented in tandem mass spectrometry.

The sets of reporter signals can be made up of reporter signals that are made up of chains or polymers. The set of reporter signals can be homosets which means that the set is made up of one type of reporter signal or that the reporter signal is made up of homochains or homopolymers. The set of reporter signals can also be a heteroset which means that the set is made up of different reporter signals or of reporter signals that are made up of different types of chains or polymers. A special type of heteroset is one in which the set is made up of different homochains or homopolymers, for example one peptide chain and one nucleic acid chain. Another special type of heteroset is one where the chains themselves are heterochains or heteropolymers. Still another type of heteroset is one which is made up of both heterochains/heteropolymers and homochains/homopolymers.

One common overall property is the property of subunit isomers. This property occurs when a set of at least two reporter signals (which typically are made up of subunit chains which are in turn made up of subunits, for example, like the relationship between a polymer and the units that make up a polymer) is made up of subunit isomers, and the set could then be called subunit isomeric or isomeric for subunits. Subunits are discussed elsewhere herein, but reporter signals can be made up of any type of chain, such as peptides or nucleic acids or polymer (general) which are in turn made up of subunits for example amino acids and phosphonucleosides, and mers (general) respectively. Within each type of subunit there are typically multiple members that are all the same type of subunit, but differ. For example, within the subunit type “amino acids,” there are many members, for example, ala, tyr, and ser, or any other combination of amino acids.

When a set of reporter signals is subunit isomeric or is made up of subunit isomers this means that each individual of the set is a subunit isomer of every other individual subunit in the set. Isomer or isomeric means that the makeup of the subunits forming the subunit chain (i.e., distribution or array) is the same but the overall connectivity of the subunits, forming the chain, is different. Thus, for example, a first reporter signal could be the chain, ala-ser-lys-gln, a second reporter signal could be the chain ala-lys-ser-gln, and a third reporter signal could be the chain ala-ser-lys-pro. If a set of reporter signals was made that contained the first reporter signal and the second reporter signal, the set would be subunit isomeric because the first reporter signal and the second reporter signal have the same makeup, i.e. each has one ala, one ser, one lys, and one gln, but each chain has a different connectivity. If, however, the set of reporter signals were made which contained the first, second, and third reporter signals the set would not be isomeric because the make up of each chain would not be the same because the first and second chains do not have a pro and the third chain does not have a gln.

Another illustration is the following: a first reporter signal could be the chain, ala-guanosine-lys-adenosine, a second reporter signal could be the chain ala-adenosine-lys-guanosine, and a third reporter signal could be the chain ala-ser-lys-pro. If a set of reporter signals was made that contained the first reporter signal and the second reporter signal, the set would be subunit isomeric because the first reporter signal and the second reporter signal have the same makeup, i.e. each has one ala, one guanosine, one lys, and one adenosine, but each chain has a different connectivity. If, however, the set of reporter signals were made which contained the first, second, and third reporter signals the set would not be isomeric because the makeup of each chain would not be the same because the first and second chains do not have a pro or a ser and the third chain does not have a guanosine or adenosine. This illustration shows that the sets can be made up of, or include, heterochains and still be considered subunit isomers.

As used herein, a common property is a property shared by a set of components (such as reporter signals). That is, the components have the property “in common.” It should be understood that reporter signals in a set may have numerous properties in common. However, as used herein, the common properties of reporter signals referred to are only those used in the disclosed method to distinguish and/or separate the reporter signals sharing the common property from molecules that lack the common property.

Reporter signals in a set can be fragmented, decomposed, reacted, derivatized, or otherwise modified or altered to distinguish the different reporter signals in the set.

Differential distribution of mass in the fragments of the reporter signals can be accomplished in a number of ways. For example, reporter signals of the same nominal structure (for example, peptides having the same amino acid sequence), can be made with different distributions of heavy isotopes, such as deuterium (²H), tritium (³H) ¹⁷O, ¹⁸O, ¹³C, or ¹⁴C. In some embodiments, stable isotopes are used. All reporter signals in the set would have the same number of a given heavy isotope, but the distribution of these would differ for different reporter signals. An example of such a set of reporter signals is A*G*SLDPAGSLR, A*GSLDPAG*SLR, and AGSLDPA*G*SLR (SEQ ID NO:2), where the asterisk indicates at least one heavy isotope substituted amino acid. For a singly charged parent ion and, following fragmentation at the scissile DP bond, one predominantly charged daughter, there are three distinguishable primary daughter ions, PAGSLR⁺, PAG*SLR⁺, PA*G*SLR⁺ (amino acids 6-11 of SEQ ID NO:2).

Similarly, reporter signals of the same general structure (for example, peptides having the same amino acid sequence), can be made with different distributions of modifications or substituent groups, such as methylation, phosphorylation, sulphation, and use of seleno-methionine for methionine. All reporter signals in the set would have the same number of a given modification, but the distribution of these would differ for different reporter signals. An example of such a set of reporter signals is AGS*M*LDPAGSMLR, AGS*MLDPAGSM*LR, and AGS*MLDPAGS*M*LR (SEQ ID NO:3), where S* indicates phosphoserine rather than serine, and, M* indicates seleno-methionine rather than methionine. For a singly charged parent ion and, following fragmentation at the scissile DP bond, one predominantly charged daughter, there are three distinguishable primary daughter ions, PAGSMLR⁺, PAGSM*LR⁺, PAGS*M*LR⁺ (amino acids 7-13 of SEQ ID NO:3).

Reporter signals of the same nominal composition (for example, made up of the same amino acids), can be made with different ordering of the subunits or components of the reporter signal. All reporter signals in the set would have the same number of subunits or components, but the distribution of these would be different for different reporter signals. An example of such a set of reporter signals is AGSLADPGSLR (SEQ ID NO:4), ALSLADPGSGR (SEQ ID NO:5), ALSLGDPASGR (SEQ ID NO:6). For a singly charged parent ion and, following fragmentation at the scissile DP bond, one predominantly charged daughter, there are three distinguishable primary daughter ions, PGSLR⁺ (amino acids 7-11 of SEQ ID NO:4), PGSGR⁺ (amino acids 7-11 of SEQ ID NO:5), PASGR⁺ (amino acids 7-11 of SEQ ID NO:6).

Reporter signals having the same nominal composition (for example, made up of the same amino acids), can be made with a labile or scissile bond at a different location in the reporter signal. All reporter signals in the set would have the same number and order of subunits or components. Where the labile or scissile bond is present between particular subunits or components, the order of subunits or components in the reporter signal can be the same except for the subunits or components creating the labile or scissile bond. Reporter signal peptides used in reporter signal fusions may use this form of differential mass distribution. An example of such a set of reporter signals is AGSLADPGSLR (SEQ ID NO:4), AGSDPLAGSLR (SEQ ID NO:7), ADPGSLAGSLR (SEQ ID NO:8). For a singly charged parent ion and, following fragmentation at the scissile DP bond, one predominantly charged daughter, there are three distinguishable primary daughter ions, PGSLR⁺ (amino acids 7-11 of SEQ ID NO:4), PLAGSLR⁺ (amino acids 5-11 of SEQ ID NO:7), PGSLAGSLR⁺ (amino acids 3-11 of SEQ ID NO:8).

Each of these modes can be combined with one or more of the other modes to produce differential distribution of mass in the fragments of the reporter signals. These modes can provide reporter signals having significant similarities in structure. As a result, related reporter signals, although differing in mass distribution, can all be detected, bound, separated and/or sorted using antibodies or other specific binding molecules that can bind the reporter signals as well as by mass. For example, different distributions of heavy isotopes can be used in reporter signals where a labile or scissile bond is placed in different locations. Different mass distribution can be accomplished in other ways. For example, reporter signals can have a variety of modifications introduced at different positions. Some examples of useful modifications include acetylation, methylation, phosphorylation, seleno-methionine rather than methionine, sulphation. Similar principles can be used to distribute charge differentially in reporter signals. Differential distribution of mass and charge can be used together in sets of reporter signals.

Reporter signals can also contain combinations of scissile bonds and labile bonds. This allows more combinations of distinguishable signals or to facilitate detection. For example, labile bonds may be used to release the isobaric fragments, and the scissile bonds used to decode the proteins.

Selenium substitution can be used to alter the mass of reporter signals. Selenium can substitute for sulfur in methionine, resulting in the modified amino acid selenomethionine. Selenium is approximately forty seven mass units larger than sulfur. Mass spectrometry may be used to identify peptides or proteins incorporating selenomethionine and methionine at a particular ratio. Small proteins and peptides with known selenium/sulfur ratio are produced, for example, by chemical synthesis incorporating selenomethionine and methionine at the desired ratio. Larger proteins or peptides may be by produced from an E. coli expression system, or any other expression system that inserts selenomethionine and methionine at the desired ratio (Hendrickson et al., Selenomethionyl proteins produced for analysis by multiwavelength anomalous diffraction (MAD): a vehicle for direct determination of three-dimensional structure. Embo J, 9(5): 1665-72 (1990), Cowie and Cohen, Biosynthesis by Escherichia coli of active altered proteins containing selenium instead of sulfur. Biochimica et Biophysica Acta, 26:252-261 (1957), and Oikawa et al., Metalloselenonein, the selenium analogue of metallothionein: synthesis and characterization of its complex with copper ions. Proc Natl Acad Sci USA, 88(8):3057-9 (1991).

Some forms of reporter signals can include one or more affinity tags. Such affinity tags can allow the detection, separation, sorting, or other manipulation of the labeled proteins, reporter signals, or reporter signal fragments based on the affinity tag. Such affinity tags are separate from and in addition to (not the basis of) the common properties of a set of reporter signals that allows separation of reporter signals from other molecules. Rather, such affinity tags serve the different purpose of allowing manipulation of a sample prior to or as a part of the disclosed method, not the means to separate reporter signals based on the common property. Reporter signals can have none, one, or more than one affinity tag. Where a reporter signal has multiple affinity tags, the tags on a given reporter signal can all be the same or can be a combination of different affinity tags. Affinity tags also can be used to distribute mass and/or charge differentially on reporter tags following the principles described above and elsewhere herein. Affinity tags can be used with reporter signals in a manner similar to the use of affinity labels as described in PCT Application WO 00/11208.

Peptide-DNA conjugates (Olejnik et al., Nucleic Acids Res., 27(23):4626-31 (1999)), synthesis of PNA-DNA constructs, and special nucleotides such as the photocleavable universal nucleotides of WO 00/04036 can be used as reporter signals in the disclosed method. Useful photocleavable linkages are also described by Marriott and Ottl, Synthesis and applications of heterobifunctional photocleavable cross-linking reagents, Methods Enzymol. 291:155-75 (1998).

Photocleavable bonds and linkages are useful in (and for use with) reporter signals because it allows precise and controlled fragmentation of the reporter signals (for subsequent detection) and precise and controlled release of reporter signals from analytes (or other intermediary molecules) to which they are attached. A variety of photocleavable bonds and linkages are known and can be adapted for use in and with reporter signals. Recently, photocleavable amino acids have become commercially available. For example, an Fmoc protected photocleavable slightly modified phenylalanine (Fmoc-D,L-β Phe(2-NO₂)) is available (Catalog Number 0011-F; Innovachem, Tucson, Ariz.). The introduction of the nitro group into the phenylalanine ring causes the amino acid to fragment under exposure to UV light (at a wavelength of approximately 350 nm). The nitrogen laser emits light at approximately 337 nm and can be used for fragmentation. The wavelength used will not cause significant damage to the rest of the peptide.

Fmoc synthesis is a common technique for peptide synthesis and Fmoc-derivative photocleavable amino acids can be incorporated into peptides using this technique. Although photocleavable amino acids are usable in and with any reporter signal, they are particularly useful in peptide reporter signals.

Use of photocleavable bonds and linkages in and with reporter signals can be illustrated with the following examples. Materials on a blank plastic substrate (for example, a Compact Disk (CD)) may be directly measured from that surface using a MALDI source ion trap. For example, a thin section of tissue sample, flash frozen, could be applied to the CD surface. A reporter molecule (for example, an antibody with a reporter signal attached via a photocleavable linkage) can be applied to the tissue surface. Recognition of specific components within the tissue allows for some of the antibody/reporter signal conjugates to associate (excess conjugate is removed during subsequent wash steps). The reporter signal then can be released from the antibody by applying a UV light and detected directly using the MALDI ion trap instrument. For example, a peptide of sequence CF*XXXXXDPXXXXXR (SEQ ID NO:24) (which contains a reporter signal) can be attached to an antibody using a disulfide bond linkage method. Exposure to the UV source of a MALDI laser will cleave the peptide at the modified phenylalanine, F*, releasing the XXXXXDPXXXXXR reporter signal (amino acids 3-15 of SEQ ID NO:24). The reporter signal subsequently can be fragmented at the DP bond and the charged fragment detected as described elsewhere herein.

Another example of the use of photocleavable linkages with reporter signals involves DNA-peptide chimeras used as reporter molecules. Such reporter molecules are useful as probes to detect particular nucleic acid sequences. In a DNA-peptide chimera (or PNA-peptide chimera), the peptide portion can be or include a reporter signal. Placement of a photocleavable phenylalanine, for example, near the DNA peptide junction of the reporter molecule allows for the release of the reporter signal from the reporter molecule by UV light. The released reporter signal can be detected directly or fragmented and detected as described elsewhere herein. Similarly to the case of the antibody-peptide reporter molecule described above, the DNA-peptide chimera can be associated with a nucleic acid molecule present on the surface of a substrate such as a CD and the reporter signal released using the UV source of a MALDI laser.

A photocleavable linkage also can be incorporated into a reporter signal and used for fragmentation of the reporter signal in the disclosed methods. For example, a photocleavable amino acid (such as the photocleavable phenylalanine) can be incorporated at any desired position in a peptide reporter signal. A reporter signal such as XXXXXXF*XXXXXR containing photocleavable phenylalanine (F*) that is photocleavable. The reporter signal can then be fragmented using the appropriate wavelength of light and the charged fragment detected. When ionizing the reporter signal (from a surface, for example) for detection, a MALDI laser that does not cause significant photocleavage (for example, Er:YAG at 2.94 μm) can be used for ionization and a second laser (for example, Nitrogen at 337 nm) can be used to fragment the reporter signal. In this case XXXXXXFXXXXXR⁺ would be photocleaved to yield XXXXXR⁺. The second laser may intersect the reporter signal ion packet at any location. Modification to the vacuum system of a mass spectrometer for this purpose is straightforward.

The use of photocleavable linkages in reporter signals is particularly useful when the analyte (or other component) to which the reporter signal is attached could fragment at a scissile bond in a collision cell. For example, in reporter signal fusions, a protein fragment/reporter signal polypeptide could be generated that contained a scissile bond in both the protein fragment portion and the reporter signal portion. An example would be XXXXXXXXXDPXXX(XXXXXXXDPXXXXXXXR)XXXX (SEQ ID NO:25), where the sequence in parenthesis indicate the reporter signal portion and the DP dipeptides contain scissile bonds. Fragmenting this polypeptide in a collision cell could result in fragmentation at either or both of the DP bonds, thus complicating the fragment spectrum. Use of a photocleavable linkage (such as a photocleavable amino acid) in the reporter signal portion would allow specific photocleavage of the reporter signal during analysis. For example, an analogous polypeptide XXXXXXXXDPXXX(XXXXXXXF*XXXXXXXR)XXXX (SEQ ID NO:26) would allow specific photocleavage a the F* position of the reporter signal.

Multiple photocleavable bonds and/or linkages can be used in or with the same reporter signals or reporter signal conjugates (such as reporter molecules or reporter signal fusions) to achieve a variety of effects. For example, different photocleavable linkages that are cleaved by different wavelengths of light can be used in different parts of reporter signals or reporter signal conjugates to be cleaved at different stages of the method. Different fragmentation wavelengths allow sequential processing which enables, for example, the combinations of the release and fragmentation methods.

As an example, a peptide containing two photocleavable amino acids, Z (cleavage wavelength in the infrared) and F* (photocleavable phenylalanine, cleavage wavelength in UV) can be constructed of the form XZXXXXXXF*XXXXXXR where the amino terminus can be attached to an analyte or other molecule utilizing known chemistry. The result is a reporter signal/analyte conjugate (or, alternatively, a reporter molecule). The reporter signal can be released from the conjugate by exposing the conjugate to an appropriate wavelength of light (infrared in this example), thus cleaving the bond at Z. Once the parent ion is selected and stored in the ion trap, the reporter signal can be fragmented by exposing it to an appropriate wavelength of light (UV in this example) to produce the daughter ion (XXXXXXR⁺) which can be detected and quantitated.

Reporter signal calibrators are a special form of reporter signal characterized by their use in reporter signal calibration. Reporter signal calibrators can be any form of reporter signal, as described above and elsewhere herein, but are used as separate molecules that are not physically associated with analytes being assessed. Thus, reporter signal calibrators need not (or do not) have reactive groups for coupling to analytes and need not be (or are not) associated with specific binding molecules or other molecules or components described herein as being associated with reporter signals.

Reporter signal calibrators may share one or more common properties with one or more analytes. Reporter signal calibrators and analytes that share one or more common properties are referred to as a reporter signal calibrator/analyte set. When only one analyte and one reporter signal calibrator share the common property they also can be referred to as a reporter signal calibrator/analyte pair. Reporter signal calibrators and analytes in a reporter signal calibrator/analyte set are said to be matching. The common property allows a reporter signal calibrator and its matching analyte to be distinguished and/or separated from other molecules lacking one or more of the properties. In some embodiments, the reporter signal calibrators and analytes in a set have the same mass-to-charge ratio (m/z). That is, the matching reporter signal calibrators and analytes in a set are isobaric. This allows the reporter signal calibrators and analytes to be separated precisely from other molecules based on mass-to-charge ratio. Reporter signal calibrators can be fragmented, decomposed, reacted, derivatized, or otherwise modified or altered to distinguish the altered reporter signal calibrators from their matching analytes. The analytes can also be fragmented.

Non-limiting analytes for use with reporter signal calibrators are proteins, peptides, and/or protein fragments (collectively referred to for convenience as proteins). Reporter signal calibrators and proteins that share one or more common properties are referred to as a reporter signal calibrator/protein set. When only one protein and one reporter signal calibrator share the common property they also can be referred to as a reporter signal calibrator/protein pair. Reporter signal calibrators and proteins in a reporter signal calibrator/analyte set are said to be matching.

As described elsewhere herein, reporter signal calibrators can be used as standards for assessing the presence and amount of analytes in samples. For this purpose, a reporter signal calibrator designed for each analyte to be assessed can be mixed with the sample to be analyzed. Analytes and their matching reporter signal calibrators are then processed together to result in detection of both analytes and reporter signal calibrators (e.g., in their altered forms). The amount of reporter signal calibrator or altered reporter signal calibrator detected provides a standard (since the amount of reporter signal calibrator added can be known) against which the amount of analyte or altered analyte detected can be compared. This allows the amount of analyte present in the sample to be accurately gauged.

a. Analytes

The disclosed methods make use of analytes generally as objects of detection, measurement and/or analysis. Analytes can be any molecule or portion of a molecule that is to be detected, measured, or otherwise analyzed. An analyte need not be a physically separate molecule, but may be a part of a larger molecule. Analytes include biological molecules, organic molecules, chemicals, compositions, and any other molecule or structure to which the disclosed method can be adapted. It should be understood that different forms of the disclosed method are more suitable for some types of analytes than other forms of the method. Analytes are also referred to as target molecules.

Non-limiting analytes are biological molecules. Biological molecules include but are not limited to proteins, peptides, enzymes, amino acid modifications, protein domains, protein motifs, nucleic acid molecules, nucleic acid sequences, DNA, RNA, mRNA, cDNA, metabolites, carbohydrates, and nucleic acid-motifs. As used herein, “biological molecule” and “biomolecule” refer to any molecule or portion of a molecule or multi-molecular assembly or composition, that has a biological origin, is related to a molecule or portion of a molecule or multi-molecular assembly or composition that has a biological origin. Biomolecules can be completely artificial molecules that are related to molecules of biological origin.

a. Analyte Samples

Any sample from any source can be used with the disclosed method. In general, analyte samples should be samples that contain, or may contain, analytes. Examples of suitable analyte samples include cell samples, tissue samples, cell extracts, components or fractions purified from another sample, environmental samples, culture samples, tissue samples, bodily fluids, and biopsy samples. Numerous other sources of samples are known or can be developed and any can be used with the disclosed method. Non-limiting analyte samples for use with the disclosed method are samples of cells and tissues. Analyte samples can be complex, simple, or anywhere in between. For example, an analyte sample may include a complex mixture of biological molecules (a tissue sample, for example), an analyte sample may be a highly purified protein preparation, or a single type of molecule.

a. Protein Samples

Any sample from any source can be used with the disclosed method. In general, protein samples should be samples that contain, or may contain, protein molecules. Examples of suitable protein samples include cell samples, tissue samples, cell extracts, components or fractions purified from another sample, environmental samples, biofilm samples, culture samples, tissue samples, bodily fluids, and biopsy samples. Numerous other sources of samples are known or can be developed and any can be used with the disclosed method. Non-limiting protein samples for use with the disclosed method are samples of cells and tissues. Protein samples can be complex, simple, or anywhere in between. For example, a protein sample may include a complex mixture of proteins (a tissue sample, for example), a protein sample may be a highly purified protein preparation, or a single type of protein.

a. Reporter Molecules

Reporter molecules are molecules that combine a reporter signal with a specific binding molecule or decoding tag. In some embodiments, the reporter signal and specific binding molecule or decoding tag are covalently coupled or tethered to each other. As used herein, molecules are coupled when they are covalent joined, directly or indirectly. One form of indirect coupling is via a linker molecule. The reporter signal can be coupled to the specific binding molecule or decoding tag by any of several established coupling reactions. For example, Hendrickson et al., Nucleic Acids Res., 23(3):522-529 (1995) describes a suitable method for coupling oligonucleotides to antibodies.

One form of reporter molecule has a peptide nucleic acid as the decoding tag and a reporter signal peptide as the reporter signal. The peptide nucleic acid can associate with, for example, an oligonucleotide coding tag, thus associating the reporter signal peptide with the coding tag. As described elsewhere herein, coding tags can be used to labeled analytes and other molecules.

As used herein, a molecule is said to be tethered to another molecule when a loop of (or from) one of the molecules passes through a loop of (or from) the other molecule. The two molecules are not covalently coupled when they are tethered. Tethering can be visualized by the analogy of a closed loop of string passing through the hole in the handle of a mug. In general, tethering is designed to allow one or both of the molecules to rotate freely around the loop.

a. Specific Binding Molecules

As used herein, a “specific binding molecule” or “a binding molecule specific for . . . ” is a molecule that interacts specifically with a particular molecule or moiety. The molecule or moiety that interacts specifically with a specific binding molecule can be, for example, an analyte, a reporter signal, or a complex of an analyte complexed to a reporter signal. It is to be understood that the term analyte refers to both separate molecules and to portions of such molecules, such as an epitope of a protein, that interacts specifically with a specific binding molecule. One non-limiting specific binding molecule of the invention is an antibody. Either member of a receptor/ligand pair, synthetic polyamides (Dervan and Burli, Sequence-specific DNA recognition by polyamides. Curr Opin Chem Biol, 3(6):688-93 (1999); Wemmer and Dervan, Targeting the minor groove of DNA. Curr Opin Struct Biol, 7(3):355-61 (1997)), nucleic acid probes, and other molecules with specific binding affinities are further non-limitng examples of specific binding molecules, useful as the affinity portion of a reporter binding molecule.

A specific binding molecule that interacts specifically with a particular analyte is said to be specific for that analyte. For example, where the specific binding molecule is an antibody that associates with a particular antigen, the specific binding molecule is said to be specific for that antigen. The antigen is the analyte. A reporter molecule containing the specific binding molecule can also be referred to as being specific for a particular analyte. A specific binding molecule that interacts specifically with a particular reporter signal or set of reporter signals is said to be specific for that reporter signal or set of reporter signals. For example, where the specific binding molecule is an antibody that associates with a particular antigen, the specific binding molecule is said to be specific for that antigen. The antigen is the reporter signal. Non-limiting specific binding molecules include antibodies, ligands, binding proteins, receptor proteins, haptens, aptamers, carbohydrates, synthetic polyamides, peptide nucleic acids, or oligonucleotides. Non-limiting binding proteins are DNA binding proteins. Non-limiting DNA binding proteins are zinc finger motifs, leucine zipper motifs, helix-turn-helix motifs. These motifs can be combined in the same specific binding molecule.

Antibodies useful as specific binding molecules, can be obtained commercially or produced using well established methods. For example, Johnstone and Thorpe, Immunochemistry In Practice (Blackwell Scientific Publications, Oxford, England, 1987) on pages 30-85, describe general methods useful for producing both polyclonal and monoclonal antibodies. The entire book describes many general techniques and principles for the use of antibodies in assay systems. Thus, in some embodiments, a binding molecule of the invention is an antibody.

The term “antibody” is used in the broadest sense and specifically covers single anti-target monoclonal antibodies and anti-target antibody compositions with polyepitopic specificity (including binding and non-binding antibodies). The term “monoclonal antibody” as used herein refers to an antibody obtained from a population of substantially homogeneous antibodies, i.e., the individual antibodies comprising the population are identical except for possible naturally occurring mutations that may be present in minor-amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen.

The monoclonal antibodies herein include hybrid and recombinant antibodies produced by splicing a variable (including hypervariable) domain of an anti-target antibody with a constant domain (e.g., “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab)2, and Fv), so long as they exhibit the desired biological activity. (See, e.g., U.S. Pat. No. 4,816,567 and Mage & Lamoyi, in Monoclonal Antibody Production Techniques and Applications, pp. 79-97 (Marcel Dekker, Inc.), New York (1987)). Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler & Milstein, Nature 256:495 (1975), or may be made by recombinant DNA methods (U.S. Pat. No. 4,816,567). The “monoclonal antibodies” may also be isolated from phage libraries generated using the techniques described in McCafferty et al., Nature 348:552-554 (1990), for example.

“Humanized” forms of non-human (e.g., murine) antibodies are specific chimeric immunoglobulins, immunoglobulin chains or fragments thereof (such as Fv, Fab, Fab′, F(ab)2 or other antigen-binding subsequences of antibodies) which contain minimal sequence derived from non-human immunoglobulin. For the most part, humanized antibodies are human immunoglobulins (recipient antibody) in which residues from the complementary determining regions (CDRs) of the recipient antibody are replaced by residues from the CDRs of a non-human species (donor antibody) such as mouse, rat or rabbit having the desired specificity, affinity and capacity. In some instances, Fv framework region (FR) residues of the human immunoglobulin are replaced by corresponding non-human FR residues. Furthermore, the humanized antibody may comprise residues that are found neither in the recipient antibody nor in the imported CDR or FR sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR residues are those of a human immunoglobulin consensus sequence. The humanized antibody optimally also will comprise at least a portion of an immunoglobulin constant region (Fc), typically that of a human immunoglobulin.

Novel monoclonal antibodies or fragments thereof mean in principle all immunoglobulin classes such as IgM, IgG, IgD, IgE, IgA or their subclasses such as the IgG subclasses or mixtures thereof. IgG and its subclasses are useful, such as IgG1, IgG2, IgG2a, IgG2b, IgG3 or IgGM. The IgG subtypes IgG1/kappa and IgG2b/kapp are included as non-limiting embodiments. Fragments which may be mentioned are all truncated or modified antibody fragments with one or two antigen-complementary binding sites which show high binding and binding activity toward mammalian target, such as parts of antibodies having a binding site which corresponds to the antibody and is formed by light and heavy chains, such as Fv, Fab or F(ab′)2 fragments, or single-stranded fragments. Truncated double-stranded fragments such as Fv, Fab or F(ab′)2 are useful. These fragments can be obtained, for example, by enzymatic means by eliminating the Fc part of the antibody with enzymes such as papain or pepsin, by chemical oxidation or by genetic manipulation of the antibody genes. It is also possible and advantageous to use genetically manipulated, non-truncated fragments. The anti-target antibodies or fragments thereof can be used alone or in mixtures.

The antibodies, antibody fragments, mixtures or derivatives thereof advantageously have a binding affinity for target with a dissociation constant value within a log-range of from about 1×10-11 M (0.01 nM) to about 1×10-8 M (10 nM), e.g., about 1×10-10 M (0.1 nM) to about 3×10-9 M (3 nM).

The antibody genes for the genetic manipulations can be isolated, for example from hybridoma cells, in a manner known to the skilled worker. For this purpose, antibody-producing cells are cultured and, when the optical density of the cells is sufficient, the mRNA is isolated from the cells in a known manner by lysing the cells with guanidinium thiocyanate, acidifying with sodium acetate, extracting with phenol, chloroform/isoamyl alcohol, precipitating with isopropanol and washing with ethanol. cDNA is then synthesized from the mRNA using reverse transcriptase. The synthesized cDNA can be inserted, directly or after genetic manipulation, for example by site-directed mutagenesis, introduction of insertions, inversions, deletions or base exchanges, into suitable animal, fungal, bacterial or viral vectors and be expressed in appropriate host organisms. Preference is given to bacterial or yeast vectors such as pBR322, pUC18/19, pACYC184, lambda or yeast mu vectors for the cloning of the genes and expression in bacteria such as E. coli or in yeasts such as Saccharomyces cerevisiae.

The invention furthermore relates to cells that synthesize target antibodies. These include animal, fungal, bacterial cells or yeast cells after transformation as mentioned above. They are advantageously hybridoma cells or trioma cells, such as hybridoma cells. These hybridoma cells can be produced, for example, in a known manner from animals immunized with target and isolation of their antibody-producing B cells, selecting these cells for target-binding antibodies and subsequently fusing these cells to, for example, human or animal, for example, mouse mylemoa cells, human lymphoblastoid cells or heterohybridoma cells (see, e.g., Koehler et al., (1975) Nature 256: 496) or by infecting these cells with appropriate viruses to produce immortalized cell lines. Hybridoma cell lines produced by fusion are useful, such as mouse hybridoma cell lines. The hybridoma cell lines of the invention secrete particularly useful antibodies of the IgG type. The binding of the particularly useful mAb antibodies of the invention, bind with high affinity and neutralize the enzymatic activity of target.

The invention further includes derivates of these anti-target antibodies, which may retain their target-binding activity while altering one or more other properties related to their use as a pharmaceutical agent, e.g., serum stability or efficiency of production. Examples of such antitarget antibody derivatives include peptides, peptidomimetics derived from the antigen-binding regions of the antibodies, and antibodies, fragments or peptides bound to solid or liquid carriers such as polyethylene glycol, glass, synthetic polymers such as polyacrylamide, polystyrene, polypropylene, polyethylene or natural polymers such as cellulose, Sepharose or agarose, or conjugates with enzymes, toxins or radioactive or nonradioactive markers such as 3H, 123I, 125I, 131I, 32P, 35S, 14C, 51Cr, 36CI, 57Co, 55Fe, 59Fe, 90Y, 99 mTc (metastable isomer of Technetium 99), 75Se, or antibodies, fragments or peptides covalently bonded to fluorescent/chemiluminescent labels such as rhodamine, fluorescein, isothiocyanate, phycoerythrin, phycocyanin, fluorescamine, metal chelates, avidin, streptavidin or biotin.

The antibodies, antibody fragments, mixtures or derivatives thereof can be used in therapy or diagnosis directly or after coupling to solid or liquid carriers, enzymes, toxins, radioactive or nonradioactive labels or to fluorescent/chemiluminescent labels as described above. Target can be detected in a wide variety of body fluids—particularly synovial fluid.

The human target monoclonal antibody of the present invention may be obtained as follows. Those of skill in the art will recognize that other equivalent procedures for obtaining target antibodies are also available and are included in the invention.

First, a mammal is immunized with human target. Purified human target is commercially available from Sigma (catalog A6152), as well as other commercial vendors. Human target may be readily purified from human placental tissue. Furthermore, methods of immunoaffinity purification for obtaining highly purified target immunogen are known (see, e.g., Vladutiu et al., (1975) Prep. Biochem. 5: 147-59). The mammal used for raising anti-human target antibody is not restricted and may be a primate, a rodent such as mouse, rat or rabbit, bovine, sheep, goat or dog.

Next, antibody-producing cells such as spleen cells are removed from the immunized animal and are fused with myeloma cells. The myeloma cells are well-known in the art (e.g., p3×63-Ag8-653, NS-0, NS-1 or P3U1 cells may be used). The cell fusion operation may be carried out by a well-known conventional method.

The cells, after being subjected to the cell fusion operation, are then cultured in HAT selection medium so as to select hybridomas. Hybridomas, which produce antihuman monoclonal antibodies, are then screened. This screening may be carried out by, for example, sandwich ELISA (enzyme-linked immunosorbent assay) or the like in which the produced monoclonal antibodies are bound to the wells to which human target is immobilized. In this case, as the secondary antibody, an antibody specific to the immunoglobulin of the immunized animal, which is labeled with an enzyme such as peroxidase, alkaline phosphatase, glucose oxidase, beta-D-galactosidase or the like, may be employed. The label may be detected by reacting the labeling enzyme with its substrate and measuring the generated color. As the substrate, 3,3-diaminobenzidine, 2,2-diaminobis-o-dianisidine, 4-chloronaphthol, 4-aminoantipyrine, o-phenylenediamine or the like may be produced.

By the above-described operation, hybridomas, which produce anti-target antibodies, can be selected. The selected hybridomas are then cloned by the conventional limiting dilution method or soft agar method. If desired, the cloned hybridomas may be cultured on a large scale using a serum-containing or a serum free medium, or may be inoculated into the abdominal cavity of mice and recovered from ascites, thereby a large number of the cloned hybridomas may be obtained.

Methods for humanizing non-human antibodies are well known in the art. Generally, a humanized antibody has one or more amino acid residues introduced into it from a source, which is non-human. These non-human amino acid residues are often referred to as “import” residues, which are typically taken from an “import” variable domain. Humanization can be essentially performed following the method of Winter and co-workers (Jones et al., (1986) Nature 321: 522-525; Riechmann et al., (1988) Nature, 332: 323-327; and Verhoeyen et al., (1988) Science 239: 1534-1536), by substituting rodent CDRs or CDR sequences for the corresponding sequences of a human antibody. Accordingly, such “humanized” antibodies are chimeric antibodies, wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., (1993) J. Immunol., 151:2296; and Chothia and Lesk (1987) J. Mol. Biol., 196:901). Another method uses a particular framework derived from the consensus sequence of all human antibodies of a particular subgroup of light or heavy chains. The same framework may be used for several different humanized antibodies (Carter et al., (1992) Proc. Natl. Acad. Sci, (USA), 89: 4285; and Presta et al., (1993) J. Immunol., 151:2623).

It is further important that antibodies be humanized with retention of high affinity for the antigen and other favorable biological properties. To achieve this goal, according to a non-limiting method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding.

Human antibodies directed against target are also included in the invention. Such antibodies can be made, for example, by the hybridoma method. Human myeloma and mouse-human heteromyeloma cell lines for the production of human monoclonal antibodies have been described, for example, by Kozbor (1984) J. Immunol., 133, 3001; Brodeur, et al., Monoclonal Antibody Production Techniques and Applications, pp. 51-63 (Marcel Dekker, Inc., New York, 1987); and Boerner et al., (1991) J. Immunol., 147:86-95. Specific methods for the generation of such human antibodies using, for example, phage display, transgenic mouse technologies and/or in vitro display technologies, such as ribosome display or covalent display, have been described (see Osbourn et al. (2003) Drug Discov. Today 8: 845-51; Maynard and Georgiou (2000) Annu. Rev. Biomed. Eng. 2: 339-76; and U.S. Pat. Nos. 4,833,077; 5,811,524; 5,958,765; 6,413,771; and 6,537,809, the contents of each of which are incorporated herein in their entirety).

It is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such gem-line mutant mice will result in the production of human antibodies upon antigen challenge (see, e.g., Jakobovits et al., (1993) Proc. Natl. Acad. Sci. (USA), 90: 2551; Jakobovits et al., (1993) Nature, 362:255-258; and Bruggermann et al., (1993) Year in Immuno., 7:33).

Alternatively, phage display technology (McCafferty et al., (1990) Nature, 348: 552-553) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats (for review see, e.g., Johnson et al., (1993) Current Opinion in Structural Biology, 3:564-571). Several sources of V-gene segments can be used for phage display. For example, Clackson et al., ((1991) Nature, 352: 624-628) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., ((1991) J. Mol. Biol., 222:581-597, or Griffith et al., (1993) EMBO J., 12:725-734).

In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling” (see Marks et al., (1992) Bio/Technol., 10:779-783). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the nM range. A strategy for making very large phage antibody repertoires has been described by Waterhouse et al., ((1993) Nucl Acids Res., 21:2265-2266).

Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable capable of restoring a functional antigen-binding site, i.e., the epitope governs (imprints) the choice of partner. When the process is repeated in order to replace the remaining rodent V domain, a human antibody is obtained (see PCT WO 93/06213, published 1 Apr. 1993). Unlike traditional humanization of rodent antibodies by CDR grafting, this technique provides completely human antibodies, which have no framework or CDR residues of rodent origin.

In further embodiments, the binding molecule of the invention can be specific for a reporter signal target based on the properties of the reporter signal target. One non-limiting example of such a property is a his tag, which includes at least 4 or more or at least 6 or more histidine amino acid residues in a row. These his tags specifically interact with metal ions, such as Co²⁺ and Ni²⁺ Co²⁺ and Ni²⁺ are contained within TALON™ Resin (commercially available from, for example, BD Biosciences, San Jose, Calif.) and an Ni-NTA resin (commercially available from, for example, Qiagen, Venlo, The Netherlands), respectively.

Binding molecules also include DNA binding proteins (e.g., zinc finger motifs, leucine zipper motifs, helix-turn-helix motifs). These motifs can be combined in the same specific binding molecule. Properties of zinc fingers, zinc finger motifs, and their interactions, are described by Nardelli et al., Zinc finger-DNA recognition: analysis of base specificity by site-directed mutagenesis. Nucleic Acids Res, 20(16):4137-44 (1992), Jamieson et al., In vitro selection of zinc fingers with altered DNA-binding specificity. Biochemistry, 33(19):5689-95 (1994), Chandrasegaran and Smith, Chimeric restriction enzymes: what is next? Biol Chem, 380(7-8):841-8 (1999), and Smith et al., A detailed study of the substrate specificity of a chimeric restriction enzyme. Nucleic Acids Res, 27(2):674-81 (1999). Thus, if a reporter signal includes a zinc finger, a specific binding molecule of the invention may be a molecule that specifically binds to zinc finger motifs.

One form of specific binding molecule or capture tag is an oligonucleotide or oligonucleotide derivative. Such specific binding molecules or capture tags are designed for and used to detect specific nucleic acid sequences. Thus, the analyte or reporter signal for oligonucleotide specific binding molecules are nucleic acid sequences. The analyte or reporter signal can be a nucleotide sequence within a larger nucleic acid molecule. An oligonucleotide specific binding molecule can be any length that supports specific and stable hybridization between the reporter binding probe and the analyte or reporter signal. For this purpose, a length of 10 to 40 nucleotides is useful, with an oligonucleotide specific binding molecule 16 to 25 nucleotides long being particularly useful. In some embodiments, the oligonucleotide specific binding molecule is a peptide nucleic acid. Peptide nucleic acid forms a stable hybrid with DNA. This allows a peptide nucleic acid specific binding molecule to remain firmly adhered to the target sequence during subsequent amplification and detection operations.

This useful effect can also be obtained with oligonucleotide specific binding molecules or capture tags by making use of the triple helix chemical bonding technology described by Gasparro et al., Nucleic Acids Res., 22(14):2845-2852 (1994). Briefly, the oligonucleotide specific binding molecule is designed to form a triple helix when hybridized to a target sequence. This is accomplished generally as known, e.g., by selecting either a primarily homopurine or primarily homopyrimidine target sequence. The matching oligonucleotide sequence which constitutes the specific binding molecule will be complementary to the selected target sequence and thus be primarily homopyrimidine or primarily homopurine, respectively. The specific binding molecule (corresponding to the triple helix probe described by Gasparro et al.) contains a chemically linked psoralen derivative. Upon hybridization of the specific binding molecule to a target sequence, a triple helix forms. By exposing the triple helix to low wavelength ultraviolet radiation, the psoralen derivative mediates cross-linking of the probe to the target sequence.

Additional binding molecules of the invention include nucleotides, lectins, protein interaction domains, dyes, synthetics peptides and peptide analogs, and other biomolecules and biomimetics. Indeed any compound that is capable of forming a stable complex with the target reporter signal under assay conditions may be used as a target binding molecule. In general, there are a wide variety of target binding molecules suitable for use in embodiments of the subject invention. Many of the groups fall into one of the following interaction categories: protein-protein interaction; organic molecule or moiety-protein interaction; sugar-protein interaction; nucleic acid-protein interaction; nucleic acid-nucleic acid interaction; and chelating metal-ligand interaction. Appropriate target binding molecules belonging to any of these categories may be identified using suitable affinity selection and/or combinatorial chemistry methods known in the art.

For example, combinatorial chemistry can be used to identify a suitable peptide or organic molecule or moiety that binds to the target protein. The target protein can be immobilized on a suitable affinity matrix under conditions sufficient to bind the protein to the matrix, and is contacted with one or more candidate binding molecules (e.g., a mixture of peptides or compounds of a library) to be tested, under suitable binding conditions. Agents that can be assayed for binding to target polypeptides include but are not limited to small organic molecules, such as those that are commercially available—often as part of large combinatorial chemistry compound ‘libraries’—from companies such as Sigma-Aldrich (St. Louis, Mo.), Arqule (Woburn, Mass.), Enzymed (Iowa City, Iowa), Maybridge Chemical Co.(Trevillett, Cornwall, UK), MDS Panlabs (Bothell, Wash.), Pharmacopeia (Princeton, N.J.), and Trega (San Diego, Calif.). Particularly useful small organic molecules for screening using these assays are usually less than 10K molecular weight. Agents including natural products, inorganic chemicals, and biologically active materials such as proteins and toxins can also be assayed using these methods for the ability to bind to target polypeptides.

Next, the affinity matrix with bound target protein can be washed with a suitable wash buffer to remove unbound candidate binding molecules and non-specifically bound candidate binding molecules. Those agents which remain bound can be released by contacting the affinity matrix with the target protein bound thereto with a suitable elution buffer. Wash buffer can be formulated to permit binding of the target protein to the affinity matrix, without significantly disrupting binding of specifically bound binding molecules. In this aspect, elution buffer can be formulated to permit retention of the target protein by the affinity matrix, but can be formulated to interfere with binding of the candidate binding molecules to the target portion of the fusion protein. For example, a change in the ionic strength or pH of the elution buffer can lead to release of specifically bound agent, or the elution buffer can comprise a release component or components designed to disrupt binding of specifically bound agent to the target protein.

Immobilization can be performed prior to, simultaneous with, or after, contacting the fusion protein with candidate binding molecule, as appropriate. Various permutations of the method are possible, depending upon factors such as the candidate molecules tested, the affinity matrix-ligand pair selected, and elution buffer formulation. For example, after the wash step, target protein with binding molecule molecules bound thereto can be eluted from the affinity matrix with a suitable elution buffer (a matrix elution buffer, such as glutathione for a GST fusion). Where the target protein comprises a cleavable linker, such as a thrombin cleavage site, cleavage from the affinity ligand can release a portion of the target with the candidate agent bound thereto. Bound agent molecules can then be released from the target protein or its cleavage product by an appropriate method, such as extraction.

One or more candidate binding molecules can be tested simultaneously. Where a mixture of candidate binding molecules is tested, those found to bind by the foregoing processes can be separated (as appropriate) and identified by suitable methods (e.g., PCR, sequencing, chromatography). Large libraries of candidate binding molecules produced by combinatorial chemical synthesis or by other methods can be tested (see e.g., Ohlmeyer, et al. (1993) Proc. Natl. Acad. Sci. USA 90:10922 10926 and DeWitt, et al. (1993) Proc. Natl. Acad. Sci. USA 90: 6909-6913, relating to tagged compounds; see also U.S. Pat. Nos. 5,010,175, 5,182,366; and No. 4,833,092). Where binding molecules selected from a combinatorial library by the present method carry unique tags, identification of individual biomolecules by chromatographic methods is possible. Where binding molecules do not carry tags, chromatographic separation, followed by mass spectrometry to ascertain structure, can be used to identify binding molecules selected by the method, for example.

There are a number of different libraries used for the identification of small molecule binding molecules, including: (1) chemical libraries, (2) natural product libraries, and (3) combinatorial libraries comprised of random peptides, oligonucleotides or organic molecules. Chemical libraries consist of random chemical structures, some of which are analogs of known compounds or analogs of compounds that have been identified as “hits” or “leads” in other drug discovery screens, some of which are derived from natural products, and some of which arise from non-directed synthetic organic chemistry. Natural product libraries are collections of microorganisms, animals, plants, or marine organisms that are used to create mixtures for screening by: (1) fermentation and extraction of broths from soil, plant or marine microorganisms or (2) extraction of plants or marine organisms. Natural product libraries include polyketides, non-ribosomal peptides, and variants (non-naturally occurring) thereof (for a review (see Cane et al. (1998) Science 282: 63-68). Combinatorial libraries are composed of large numbers of peptides, oligonucleotides, or organic compounds as a mixture. These libraries are relatively easy to prepare by traditional automated synthesis methods, PCR, cloning, or proprietary synthetic methods. Of particular interest are non-peptide combinatorial libraries. Still other libraries of interest include peptide, protein, peptidomimetic, multiparallel synthetic collection, recombinatorial, and polypeptide libraries. For a review of combinatorial chemistry and libraries created therefrom (see Myers (1997) Curr. Opin. Biotechnol. 8: 701-707). Identification of target binding molecules through use of the various libraries described herein permits modification of the candidate “hit” (or “lead”) to optimize the capacity of the “hit” to bind the target.

J. Mass Spectrometers

The disclosed methods can make use of mass spectrometers for analysis of reporter signals, altered forms of reporters signals, and various analytes and analyte fragments. Mass spectrometers are generally available and such instruments and their operations are known to those of skill in the art. Fractionation systems integrated with mass spectrometers are commercially available, exemplary systems include liquid chromatography (LC) and capillary electrophoresis (CE).

The principle components of a mass spectrometer include: (a) one or more sources, (b) one or more analyzers and/or cells, and (c) one or more detectors. Types of sources include Electrospray Ionization (ESI) and Matrix Assisted Laser Desorption Ionization (MALDI). Types of analyzers and cells include quadrupole mass filter, hexapole collision cell, ion cyclotron trap, and Time-of-Flight (TOF). Types of detectors include Multichannel Plates (MCP) and ion multipliers. A non-limiting mass spectrometer for use with the disclosed method is described by Krutchinsky et al., Rapid Automatic Identification of Proteins Utilizing a Novel MALDI-Ion Trap Mass Spectrometer, Abstract of the 49^{th} ASMS Conference on Mass Spectrometry and Allied Topics (May 27-31, 2001), The Rockefeller University, New York, N.Y.

Mass spectrometers with more than one analyzer/cell are known as tandem mass spectrometers. There are two types of tandem mass spectrometers, as well as hybrids and combinations of these types: “tandem in space” spectrometers and “tandem in time” spectrometers. Tandem mass spectrometers where the ions traverse more than one analyzer/cell are known as tandem in space mass spectrometers. Tandem in space spectrometers utilize spatially ordered elements and act upon the ions in turn as the ions pass through each element. Tandem mass spectrometers where the ions remain primarily in one analyzer/cell are known as tandem in time mass spectrometers. Tandem in time spectrometers utilize temporally ordered manipulations on the ions as the ions are contained in a space. Hybrid systems and combinations of these types are known. The ability to select a particular mass-to-charge ratio of interest in a mass analyzer is typically characterized by the resolution (reported as the centroid mass-to-charge divided by the full width at half maximum of the selected ions of interest). Thus resolution is an indicator of the narrowness of the ion mass-to-charge distribution passed through the analyzer to the detector. Reference to such resolution is generally noted herein by referring to the ability of a mass spectrometer to pass only a narrow range of mass-to-charge ratios.

There are two types of tandem mass spectrometers, as well as hybrids and combinations of these types: “tandem in space” spectrometers and “tandem in time” spectrometers. Tandem in space spectrometers utilize spatially ordered elements and act upon the ions in turn as the ions pass through each element. Tandem in time spectrometers utilize temporally ordered manipulations on the ions as the ions are contained in a space.

A non-limiting form of mass spectrometer for use in the disclosed methods is a tandem mass spectrometer, such as a tandem in space tandem mass spectrometer. As an example of the use of a tandem in space class of instrument, the isobaric reporter signals can be first passed through a filtering quadrupole, the reporter signals are fragmented (e.g., in a collision cell), and the fragments are distinguished and detected in a time-of-flight (TOF) stage. In such an instrument the sample is ionized in the source (for example, in a MALDI ion source) to produce charged ions. In some embodiments, the ionization conditions are such that primarily a singly charged parent ion is produced. A first quadrupole, Q0, is operated in radio frequency (RF) mode only and acts as an ion guide for all charged particles. The second quadrupole, Q1, is operated in RF+DC mode to pass only a narrow range of mass-to-charge ratios (that includes the mass-to-charge ratio of the reporter signals). This quadrupole selects the mass-to-charge ratio of interest. Quadrupole Q2, surrounded by a collision cell, is operated in RF only mode and acts as ion guide. The collision cell surrounding Q2 can be filled to appropriate pressure with a gas to fracture the input ions by collisionally induced dissociation when fragmentation of the reporter signals is desired. The collision gas is, in some embodiments, chemically inert, but reactive gases can also be used. Non-limiting molecular systems utilize reporter signals that contain scissile bonds, labile bonds, or combinations, such that these bonds will be preferentially fractured in the Q2 collision cell.

Tandem instruments capable of MSN can be used with the disclosed method. As an example consider; a method where one selects a set of molecules using a first stage filter (MS), photocleaves these molecules to yield a set of reporter signals, selects these reporter signals using a second stage (MS/MS), alters these reporter signals by collisional fragmentation, detects by time of flight (MS3). Many other combinations are possible and the disclosed method can be adapted for use with such systems. For example, extension to more stages, or analysis of reporter signal fragments is within the skill of those in the art.

K. Capture Arrays and Sample Arrays

A capture array (also referred to herein as an array) includes a plurality of capture tags immobilized on a solid-state substrate, e.g., at identified or predetermined locations on the solid-state substrate. In this context, plurality of capture tags refers to a multiple capture tags each having a different structure. In some embodiments, each predetermined location on the array (referred to herein as an array element) has one type of capture tag (that is, all the capture tags at that location have the same structure). Each location will have multiple copies of the capture tag. The spatial separation of capture tags of different structure or different samples in the array allows separate detection and identification of analytes that become associated with the capture tags. If a decoding tag or a reporter signal is detected at a given location in an array, it indicates that the analyte corresponding to that array element (e.g., a reporter signal) was present in the target sample.

A non-limiting form of sample array is a tissue array, where there are small tissue samples on a substrate. Such tissue microarrays exist, and are used, for example, in a cohort to study breast cancer. The disclosed method can be used, for example, to probe multiple analytes in multiple samples. Sample arrays can be, for example, labeled with different reporter signals, the whole support then introduced into source region of a mass spec, and sampled by MALDI.

Solid-state substrates for use in capture and/or sample arrays can include any solid material to which capture tags or samples can be coupled, directly or indirectly. This includes materials such as acrylamide, cellulose, nitrocellulose, glass, polystyrene, polyethylene vinyl acetate, polypropylene, polymethacrylate, polyethylene, polyethylene oxide, glass, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, and polyamino acids. Solid-state substrates can have any useful form including thin films or membranes, beads, bottles, dishes, disks, compact disks, fibers, optical fibers, woven fibers, shaped polymers, particles and microparticles. A non-limiting form for a solid-state substrate is a compact disk.

It should be noted that it is not required that a given capture or sample array be a single unit or structure. The set of capture tags or samples may be distributed over any number of solid supports. For example, at one extreme, each capture tag or each sample may be immobilized in a separate reaction tube or container. Arrays may be constructed upon non permeable or permeable supports of a wide variety of support compositions such as those described above. The array spot sizes and density of spot packing vary over a tremendous range depending upon the process(es) and material(s) used.

Methods for immobilizing samples, sample components, antibodies and other proteins to substrates are well established. Immobilization can be accomplished by attachment, for example, to aminated surfaces, carboxylated surfaces or hydroxylated surfaces using standard immobilization chemistries. Examples of attachment agents are cyanogen bromide, succinimide, aldehydes, tosyl chloride, avidin-biotin, photocrosslinkable agents, epoxides and maleimides. non-limiting attachment agent is glutaraldehyde. These and other attachment agents, as well as methods for their use in attachment, are described in Protein immobilization: fundamentals and applications, Richard F. Taylor, ed. (M. Dekker, New York, 1991), Johnstone and Thorpe, Immunochemistry In Practice (Blackwell Scientific Publications, Oxford, England, 1987) pages 209-216 and 241-242, and Immobilized Affinity Ligands, Craig T. Hermanson et al., eds. (Academic Press, New York, 1992). Antibodies can be attached to a substrate by chemically cross-linking a free amino group on the antibody to reactive side groups present within the substrate. For example, antibodies may be chemically cross-linked to a substrate that contains free amino or carboxyl groups using glutaraldehyde or carbodiimides as cross-linker agents. In this method, aqueous solutions containing free antibodies are incubated with the solid-state substrate in the presence of glutaraldehyde or carbodiimide. For crosslinking with glutaraldehyde the reactants can be incubated with 2% glutaraldehyde by volume in a buffered solution such as 0.1 M sodium cacodylate at pH 7.4. Other standard immobilization chemistries are known by those of skill in the art.

Methods for immobilization of oligonucleotides to solid-state substrates are well established. Oligonucleotide capture tags can be coupled to substrates using established coupling methods. For example, suitable attachment methods are described by Pease et al., Proc. Natl. Acad. Sci. USA 91(11):5022-5026 (1994), Khrapko et al., Mol Biol (Mosk) (USSR) 25:718-730 (1991), U.S. Pat. No. 5,871,928 to Fodor et al., U.S. Pat. No. 5,654,413 to Brenner, U.S. Pat. No. 5,429,807, and U.S. Pat. No. 5,599,695 to Pease et al. A method for immobilization of 3′-amine oligonucleotides on casein-coated slides is described by Stimpson et al., Proc. Natl. Acad. Sci. USA 92:6379-6383 (1995). A non-limiting method of attaching oligonucleotides to solid-state substrates is described by Guo et al., Nucleic Acids Res. 22:5456-5465 (1994).

Planar array technology has been utilized for many years (Shalon, D., S. J. Smith, and P. O. Brown, A DNA microarray system for analyzing complex DNA samples using two-color fluorescent probe hybridization. Genome Res, 1996. 6(7): p. 639-45, Singh-Gasson, S., et al., Maskless fabrication of light-directed oligonucleotide microarrays using a digital micromirror array. Nat Biotechnol, 1999. 17(10): p. 974-8, Southern, E. M., U. Maskos, and J. K. Elder, Analyzing and comparing nucleic acid sequences by hybridization to arrays of oligonucleotides: evaluation using experimental models. Genomics, 1992. 13(4): p. 1008-17, Nizetic, D., et al., Construction, arraying, and high-density screening of large insert libraries of human chromosomes X and 21: their potential use as reference libraries. Proc Natl Acad Sci USA, 1991. 88(8): p. 3233-7, Van Oss, C. J., R. J. Good, and M. K. Chaudhury, Mechanism of DNA (Southern) and protein (Western) blotting on cellulose nitrate and other membranes. J Chromatogr, 1987. 391(1): p. 53-65, Ramsay, G., DNA chips: state-of-the art. Nat Biotechnol, 1998. 16(1): p. 40-4, Schena, M., et al., Parallel human genome analysis: microarray-based expression monitoring of 1000 genes. Proc Natl Acad Sci USA, 1996. 93(20): p. 10614-9, Lipshutz, R. J., et al., High density synthetic oligonucleotide arrays. Nat Genet, 1999. 21(1 Suppl): p. 20-4, Pease, A. C., et al., Light-generated oligonucleotide arrays for rapid DNA sequence analysis. Proc Natl Acad Sci US A, 1994. 91(11): p. 5022-6, Maier, E., et al., Application of robotic technology to automated sequence fingerprint analysis by oligonucleotide hybridisation. J Biotechnol, 1994. 35(2-3): p. 191-203, Vasiliskov, A. V., et al., Fabrication of microarray of gel-immobilized compounds on a chip by copolymerization. Biotechniques, 1999. 27(3): p. 592-4, 596-8, 600 passim, and Yershov, G., et al., DNA analysis and diagnostics on oligonucleotide microchips. Proc Natl Acad Sci USA, 1996. 93(10): p. 4913-8).

Oligonucleotide capture tags in arrays can also be designed to have similar hybrid stability. This would make hybridization of fragments to such capture tags more efficient and reduce the incidence of mismatch hybridization. The hybrid stability of oligonucleotide capture tags can be calculated using known formulas and principles of thermodynamics (see, for example, Santa Lucia et al., Biochemistry 35:3555-3562 (1996); Freier et al., Proc. Natl. Acad. Sci. USA 83:9373-9377 (1986); Breslauer et al., Proc. Natl. Acad. Sci. USA 83:3746-3750 (1986)). The hybrid stability of the oligonucleotide capture tags can be made more similar (a process that can be referred to as smoothing the hybrid stabilities) by, for example, chemically modifying the capture tags (Nguyen et al., Nucleic Acids Res. 25(15):3059-3065 (1997); Hohsisel, Nucleic Acids Res. 24(3):430-432 (1996)). Hybrid stability can also be smoothed by carrying out the hybridization under specialized conditions (Nguyen et al., Nucleic Acids Res. 27(6): 1492-1498 (1999); Wood et al., Proc. Natl. Acad. Sci. USA 82(6):1585-1588 (1985)).

Another means of smoothing hybrid stability of the oligonucleotide capture tags is to vary the length of the capture tags. This would allow adjustment of the hybrid stability of each capture tag so that all of the capture tags had similar hybrid stabilities (to the extent possible). Since the addition or deletion of a single nucleotide from a capture tag will change the hybrid stability of the capture tag by a fixed increment, it is understood that the hybrid stabilities of the capture tags in a capture array will not be equal. For this reason, similarity of hybrid stability as used herein refers to any increase in the similarity of the hybrid stabilities of the capture tags (or, put another way, any reduction in the differences in hybrid stabilities of the capture tags).

The efficiency of hybridization and ligation of oligonucleotide capture tags to sample fragments can also be improved by grouping capture tags of similar hybrid stability in sections or segments of a capture array that can be subjected to different hybridization conditions. In this way, the hybridization conditions can be optimized for particular classes of capture tags.

L. Capture Tags

A capture tag is any compound that can be used to capture or separate compounds or complexes having the capture tag. In some embodiments, a capture tag is a compound that interacts specifically with a particular molecule or moiety. In some embodiments, the molecule or moiety that interacts specifically with a capture tag is an analyte. It is to be understood that the term analyte refers to both separate molecules and to portions of such molecules, such as an epitope of a protein, that interacts specifically with a capture tag. Antibodies, either member of a receptor/ligand pair, synthetic polyamides (Dervan and Burli, Sequence-specific DNA recognition by polyamides. Curr Opin Chem Biol, 3(6):688-93 (1999); Wemmer and Dervan, Targeting the minor groove of DNA. Curr Opin Struct Biol, 7(3):355-61 (1997)), nucleic acid probes, and other molecules with specific binding affinities are examples of capture tags.

A capture tag that interacts specifically with a particular analyte is said to be specific for that analyte. For example, where the capture tag is an antibody that associates with a particular antigen, the capture tag is said to be specific for that antigen. The antigen is the analyte. Capture tags are, for example, antibodies, ligands, binding proteins, receptor proteins, haptens, aptamers, carbohydrates, synthetic polyamides, peptide nucleic acids, or oligonucleotides. Non-limiting binding proteins are DNA binding proteins. Non-limiting DNA binding proteins are zinc finger motifs, leucine zipper motifs, helix-turn-helix motifs. These motifs can be combined in the same capture tag.

Antibodies useful as the affinity portion of reporter binding agents, can be obtained commercially or produced using well established methods. For example, Johnstone and Thorpe, Immunochemistry In Practice (Blackwell Scientific Publications, Oxford, England, 1987) on pages 30-85, describe general methods useful for producing both polyclonal and monoclonal antibodies. The entire book describes many general techniques and principles for the use of antibodies in assay systems.

Properties of zinc fingers, zinc finger motifs, and their interactions, are described by Nardelli et al., Zinc finger-DNA recognition: analysis of base specificity by site-directed mutagenesis. Nucleic Acids Res, 20(16):4137-44 (1992), Jamieson et al., In vitro selection of zinc fingers with altered DNA-binding specificity. Biochemistry, 33(19):5689-95 (1994), Chandrasegaran and Smith, Chimeric restriction enzymes: what is next? Biol Chem, 380(7-8):841-8 (1999), and Smith et al., A detailed study of the substrate specificity of a chimeric restriction enzyme. Nucleic Acids Res, 27(2):674-81 (1999).

M. Decoding Tags

Decoding tags are any molecule or moiety that can be associated with coding tags, directly or indirectly. Decoding tags are associated with reporter signals (making up a reporter molecule) to allow indirect association of the reporter signals with an analyte. Decoding tags may be oligonucleotides, carbohydrates, synthetic polyamides, peptide nucleic acids, antibodies, ligands, proteins, haptens, zinc fingers, aptamers, or mass labels.

Non-limiting decoding tags are molecules capable of hybridizing specifically to an oligonucleotide decoding tag. One exemplary coding tag is a peptide nucleic acid decoding tags. Oligonucleotide or peptide nucleic acid decoding tags can have any arbitrary sequence. The only requirement is hybridization to coding tags. The decoding tags can each be any length that supports specific and stable hybridization between the coding tags and the decoding tags. For this purpose, a length of 10 to 35 nucleotides is useful, such as a decoding tag of 15 to 20 nucleotides in length.

Reporter molecules containing decoding tags preferably are capable of being released by matrix-assisted laser desorption-ionization (MALDI) in order to be separated and identified by time-of-flight (TOF) mass spectroscopy, or by another detection technique. A decoding tag may be any oligomeric molecule that can hybridize to a coding tag. For example, a decoding tag can be a DNA oligonucleotide, an RNA oligonucleotide, or a peptide nucleic acid (PNA) molecule. In some embodiments, decoding tags are PNA molecules.

N. Coding Tags

Coding tags are molecules or moieties with which decoding tags can associate. Coding tags can be any type of molecule or moiety that can serve as a target for decoding tag association. Non-limiting coding tags are oligomers, oligonucleotides, or nucleic acid sequences. Coding tags can also be a member of a binding pair, such as streptavidin or biotin, where its cognate decoding tag is the other member of the binding pair. Coding tags can also be designed to associate directly with some types of reporter signals. For example, oligonucleotide coding tags can be designed to interact directly with peptide nucleic acid reporter signals (which are reporter signals composed of peptide nucleic acid).

The oligomeric base sequences of oligomeric coding tags can include RNA, DNA, modified RNA or DNA, modified backbone nucleotide-like oligomers such as peptide nucleic acid, methylphosphonate DNA, and 2′-O-methyl RNA or DNA. Oligomeric or oligonucleotide coding tags can have any arbitrary sequence. The only requirement is association with decoding tags (e.g., by hybridization). In the disclosed method, multiple coding tags can become associated with a single analyte. The context of these multiple coding tags depends upon the technique used for signal amplification. Thus, where branched DNA is used, the branched DNA molecule includes the multiple coding tags on the branches. Where oligonucleotide dendrimers are used, the coding tags are on the dendrimer arms. Where rolling circle replication is used, multiple coding tags result from the tandem repeats of complement of the amplification target circle sequence (which includes at least one complement of the coding tag sequence). In this case, the coding tags are tandemly repeated in the tandem sequence DNA.

Oligonucleotide coding tags can each be any length that supports specific and stable hybridization between the coding tags and the decoding tags. For this purpose, a length of 10 to 35 nucleotides is useful, such as a coding tag of 15 to 20 nucleotides in length.

The branched DNA for use in the disclosed method is generally known (Urdea, Biotechnology 12:926-928 (1994), and Horn et al., Nucleic Acids Res 23:4835-4841 (1997)). As used herein, the tail of a branched DNA molecule refers to the portion of a branched DNA molecule that is designed to interact with the analyte. The tail is a specific binding molecule. In general, each branched DNA molecule should have only one tail. The branches of the branched DNA (also referred to herein as the arms of the branched DNA) contain coding tag sequences. Oligonucleotide dendrimers (or dendrimeric DNA) are also generally known (Shchepinov et al., Nucleic Acids Res. 25:4447-4454 (1997), and Orentas et al., J. Virol. Methods 77:153-163 (1999)). As used herein, the tail of an oligonucleotide dendrimer refers to the portion of a dendrimer that is designed to interact with the analyte. In general, each dendrimer should have only one tail. The dendrimeric strands of the dendrimer are referred to herein as the arms of the oligonucleotide dendrimer and contain coding tag sequences.

Coding tags can be coupled (directly or via a linker or spacer) to analytes or other molecules to be labeled. Coding tags can also be associated with analytes and other molecules to be labeled. For this purpose, coding molecules are useful. Coding molecules are molecules that can interact with an analyte and with a decoding tag. Coding molecules include a specific binding molecule and a coding tag. Specific binding molecules are described above.

O. Reporter Carriers and Coding Carriers

Reporter carriers are associations of one or more specific binding molecules, a carrier, and a plurality of reporter signals. Reporter carriers are used in the disclosed method to associate a large number of reporter signals with an analyte. Coding carriers are associations of one or more specific binding molecules, a carrier, and a plurality of coding tags. Coding carriers are used in the disclosed method to associate a large number of coding tags with an analyte. The carrier can be any molecule or structure that facilitates association of many reporter signals with a specific binding molecule. Examples include liposomes, microparticles, nanoparticles, virons, phagmids, and branched polymer structures. A general class of carriers are structures and materials designed for drug delivery. Many such carriers are known. Liposomes are a non-limiting form of carrier.

Liposomes are artificial structures primarily composed of phospholipid bilayers. Cholesterol and fatty acids may also be included in the bilayer construction. In some forms of the disclosed method, liposomes serve as carriers for arbitrary reporter signals or coding tags. By combining liposome reporter carriers, loaded with arbitrary signals or tags, with methods capable of separating a very large multiplicity of signals and tags, it becomes possible to perform highly multiplexed assays.

Liposomes, such as unilamellar vesicles, are made using established procedures that result in the loading of the interior compartment with a very large number (several thousand) of reporter signals or coding tag molecules, where the chemical nature of these molecules is well suited for detection by a preselected detection method. One specific type of reporter signal or coding tag is used, for example, for each specific type of liposome carrier.

Each specific type of liposome reporter or coding carrier is associated with a specific binding molecule. The association may be direct or indirect. An example of a direct association is a liposome containing covalently coupled antibodies on the surface of the phospholipid bilayer. An alternative, indirect association composition is a liposome containing covalently coupled DNA oligonucleotides of arbitrary sequence on its surface; these oligonucleotides are designed to recognize, by base complementarity, specific reporter molecules. The reporter molecule may comprise an antibody-DNA covalent complex, whereby the DNA portion of this complex can hybridize specifically with the complementary sequence on a liposome reporter carrier. In this fashion, the liposome reporter carrier becomes a generic reagent, which may be associated indirectly with any desired binding molecule.

The use of liposome reporter carriers can be illustrated with the following example:

1. Liposomes (e.g., unilamellar vesicles with an average diameter of 150 to 300 nanometers) are prepared using the extrusion method (Hope et al., Biochimica et Biophysica Acta, 812:55-65 (1985); MacDonald et al., Biochimica et Biophysica Acta, 1061:297-303 (1991)). Other methods for liposome preparation may be used as well.

2. A solution of an oligopeptide, at a concentration 400 micromolar, is used during the preparation of the liposomes, such that the inner volume of the liposomes is loaded with this specific oligopeptide, which will serve to identify a specific analyte of interest. A liposome with an internal diameter of 200 nanometers will contain, on the average, 960 molecules of the oligopeptide. Three separate preparations of liposomes are extruded, each loaded with a different oligopeptide. The oligopeptides are chosen such that they have the same mass-to-charge ratio but will break into fragments with different mass-to-charge ratios such that they will be readily separable by mass spectrometry.

3. The outer surface of the three liposome preparations is conjugated with specific antibodies, as follows: a) the first liposome preparation is reacted with an antibody specific for the p53 tumor suppressor; b) the second liposome preparation is reacted with an antibody specific for the Bcl-2 oncoprotein; c) the third liposome preparation is reacted with an antibody specific or the Her2/neu membrane receptor. Coupling reactions are performed using standard procedures for the covalent coupling of antibodies to molecules harboring reactive amino groups (Hendrickson et al., Nucleic Acids Research, 23:522-529 (1995); Hermanson, Bioconjugate techniques, Academic Press, pp. 528-569 (1996); Scheffold et al., Nature Medicine 1: 107-110 (2000)). In the case of the liposomes, the reactive amino groups are those present in the phosphatidyl ethanolamine moieties of the liposomes.

4. A glass slide bearing a standard formaldehyde-fixed histological section is contacted with a mixture of all three liposome preparations, suspended in a buffer containing 30 mM Tris-HCl, pH 7.6, 100 mM Sodium Chloride, 1 mM EDTA, 0.1% Bovine serum albumin, in order to allow association of the liposomes with the corresponding protein antigens present in the fixed tissue. After a one hour incubation, the slides are washed twice, for 5 minutes, with the same buffer (30 mM Tris-HCl, pH 7.6, 100 mM Sodium Chloride, 1 mM EDTA, 0.1% Bovine serum albumin). The slides are dried with a stream of air.

5. The slides are coated with a thin layer of matrix solution consisting of 10 mg/ml alpha-cyano-4-hydroxycinnamic acid, 0.1% trifluoroacetic acid in a 50:50 mixture of acetonitrile in water. The slides are dried with a stream of air.

6. The slide is placed on the surface of a MALDI plate, and introduced in a mass spectrometer such as that described in Loboda et al., Design and Performance of a MALDI-QqTOF Mass Spectrometer, in 47th ASMS Conference, Dallas, Tex. (1999), Loboda et al., Rapid Comm. Mass Spectrom. 14(12):1047-1057 (2000), Shevchenko et al., Anal. Chem., 72: 2132-2142 (2000), and Krutchinsky et al., J. Am. Soc. Mass Spectrom., 11(6):493-504 (2000).

7. Mass spectra are obtained from defined positions on the slide surface. The relative amount of each of the three peaks of reporter signal polypeptides is used to determine the relative ratios of the antigens detected by the liposome-detector complexes.

The liposome carrier method is not limited to the detection of analytes on histological sections. Cells obtained by sorting may also be used for analysis in the disclosed method (Scheffold, A., Assenmacher, M., Reiners-Schramm, L., Lauster, R., and Radbruch, A., 2000, Nature Medicine 1: 107-110).

P. Analytes

Labeled analytes (such as proteins) are analytes to which one or more reporter signals are attached. In some embodiments, the reporter signal and the analyte are covalently coupled or tethered to each other. As used herein, molecules are coupled when they are covalent joined, directly or indirectly. One form of indirect coupling is via a linker molecule. The reporter signal can be coupled to the analyte by any suitable coupling reactions. Many chemistries and techniques for coupling compounds are known and can be used to couple reporter signals to analytes. For example, coupling can be made using thiols, epoxides, nitriles for thiols, NHS esters, isothiocyantes, isothiocyanates for amines, amines, and alcohols for carboxylic acids. Where the analyte is a protein, reporter signals can be covalently coupled to proteins through a sulfur-sulfur bond between a cysteine on the protein and a cysteine on the reporter signal. Analytes can also be labeled in vivo.

As used herein, “labeled analyte” refers to analytes to which one or more reporter signals are attached. The term labeled analyte refers both to analytes attached to intact (for example, unfragmented) reporter signals and to analytes attached to modified (for example, fragmented) reporter signals. The latter form of labeled proteins are referred to as fragmented labeled analytes. Although the analyte portion of a labeled analyte can be fragmented (e.g., by digestion with an enzyme such as a protease, lipidase, or glycosidase, depending on the type of analyte), the term fragmented labeled analyte refers to a labeled analyte where the reporter signal has been fragmented. Isobaric labeled analytes are analytes of the same type that are labeled with isobaric reporter signals such that a set of the analytes has the same mass-to-charge ratio.

Q. Affinity Tags

An affinity tag is any compound included within a reporter peptide or attached to a reporter peptide that can be used to separate compounds or complexes having the affinity tag from those that do not. In some embodiments, an affinity tag is a compound, such as a ligand or hapten, that associates or interacts with another compound, such as ligand-binding molecule or an antibody, where the ligand-binding molecule or antibody is used as a capturing compound. In some embodiments, such interaction between the affinity tag and the capturing component is a specific interaction. One non-limiting affinity tag is the his tag, which includes at least 4 or more or at least 6 or more histidine amino acid residues in a row. These his tags specifically interact with metal ions, such as Co²⁺ and Ni²⁺. Co²⁺ and Ni²⁺ are contained within TALON™ Resin (commercially available from, for example, BD Biosciences, San Jose, Calif.) and an Ni-NTA resin (commercially available from, for example, Qiagen, Venlo, The Netherlands), respectively. Another non-limiting set of affinity tags include biotin and avidin (or streptavidin). In this case, either of biotin or avidin is included as an affinity tag within the reporter signal, and the other of biotin or avidin (i.e., biotin if avidin is the affinity tag) is used as the capturing component. Additional non-limiting affinity tags include ligands or haptens (antibodies used as capturing compounds), binding protein targets (respective binding protein used as capturing compound), receptor protein ligands (respective receptor proteins used as capturing compounds), aptamers, carbohydrates, synthetic polyamides, or oligonucleotides. Binding protein affinity tags include DNA binding proteins (e.g., zinc finger motifs, leucine zipper motifs, helix-turn-helix motifs). These motifs can be combined in the same specific binding molecule.

Affinity tags, described in the context of nucleic acid probes, are described by Syvnen et al., Nucleic Acids Res., 14:5037 (1986). For example, the biotin affinity tag can be incorporated into nucleic acids. In the disclosed method, affinity tags incorporated into reporter signals can allow the reporter signals to be captured by, adhered to, or coupled to a substrate (e.g., a substrate coated with avidin or streptavidin). Such capture allows separation of reporter signals from other molecules, simplified washing and handling of reporter signals, and allows automation of all or part of the method.

Zinc fingers can also be used as affinity tags. Properties of zinc fingers, zinc finger motifs, and their interactions, are described by Nardelli et al., Zinc finger-DNA recognition: analysis of base specificity by site-directed mutagenesis. Nucleic Acids Res, 20(16):4137-44 (1992), Jamieson et al., In vitro selection of zinc fingers with altered DNA-binding specificity. Biochemistry, 33(19):5689-95 (1994), Chandrasegaran, S. and J. Smith, Chimeric restriction enzymes: what is next? Biol Chem, 380(7-8):841-8 (1999), and Smith et al., A detailed study of the substrate specificity of a chimeric restriction enzyme. Nucleic Acids Res, 27(2):674-81 (1999).

Capturing reporter signals on a substrate, if desired, may be accomplished in several ways. In one embodiment, affinity docks are adhered or coupled to the substrate. Affinity docks are compounds or moieties that mediate adherence of a reporter signal by associating or interacting with an affinity tag on the reporter signal. Affinity docks immobilized on a substrate allow capture of the reporter signals on the substrate. Such capture provides a convenient means of washing away molecules that might interfere with subsequent steps. Captured reporter signals can also be released from the substrate. This can be accomplished by dissociating the affinity tag or by breaking a photocleavable linkage between the reporter signal and the substrate.

Substrates for use in the disclosed method can include any solid material to which reporter signals can be adhered or coupled. Examples of substrates include, but are not limited to, materials such as acrylamide, cellulose, nitrocellulose, glass, silicon, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, and polyamino acids. Substrates can have any useful form including thin films or membranes, beads, bottles, dishes, fibers, optical fibers, woven fibers, shaped polymers, particles, compact disks, and microparticles.

R. Vectors and Expression Sequences

Gene transfer can be obtained using direct transfer of genetic material, in but not limited to, plasmids, viral vectors, viral nucleic acids, phage nucleic acids, phages, cosmids, and artificial chromosomes, or via transfer of genetic material in cells or carriers such as cationic liposomes. Such methods are well known in the art and readily adaptable for use in the method described herein. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991).

As used herein, plasmid or viral vectors are agents that transport the gene into the cell without degradation and include a promoter yielding expression of the gene in the cells into which it is delivered. In a some embodiments, vectors are derived from either a virus or a retrovirus. Non-limiting viral vectors are Adenovirus, Adeno-associated virus, Herpes virus, Vaccinia virus, Polio virus, AIDS virus, neuronal trophic virus, Sindbis and other RNA viruses, including these viruses with the HIV backbone. Also useful are any viral families which share the properties of these viruses which make them suitable for use as vectors. Non-limiting retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. In some embodiments, a viral vector which has been engineered so as to suppress the immune response of the host organism, elicited by the viral antigens, is used. Non-limiting vectors of this type will carry coding regions for Interleukin 8 or 10.

Viral vectors have higher transaction (ability to introduce genes) abilities than do most chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/□romoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

a. Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the Trna primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. Either positive or negative selectable markers along with other genes may be included in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

b. Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A non-limiting viral vector is one based on an adenovirus which has had the E1 gene removed and these virons are generated in a cell line such as the human 293 cell line. In another embodiment both the E1 and E3 genes are removed from the adenovirus genome.

Another type of viral vector is based on an adeno-associated virus (AAV). This defective parvovirus is a non-limiting vector because it can infect many cell types and is nonpathogenic to humans. AAV type vectors can transport about 4 to 5 kb and wild type AAV is known to stably insert into chromosome 19. Vectors which contain this site specific integration property are useful. One embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

c. Viral Promoters and Enhancers

Non-limiting promoters controlling transcription from vectors in mammalian host cells may be obtained from various sources, for example, the genomes of viruses such as: polyoma, Simian Virus 40 (SV40), adenovirus, retroviruses, hepatitis-B virus and cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell. Bio. 3: 1108 (1983)) to the transcription unit. Furthermore, enhancers can be within an intron (Baneji, J. L. et al., Cell 33: 729 (1983)) as well as within the coding sequence itself (Osborne, T. F., et al., Mol. Cell. Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus. Non-limiting examples are the SV40 enhancer on the late side of the replication origin (bp 100-270), the cytomegalovirus early promoter enhancer, the polyoma enhancer on the late side of the replication origin, and adenovirus enhancers.

The □romoter and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In some embodiments, the promoter and/or enhancer region act as a constitutive promoter and/or enhancer to maximize expression of the region of the transcription unit to be transcribed. The promoter and/or enhancer region be active in all eukaryotic cell types. A promoter of this type is the CMV promoter (650 bases). Other promoters include SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect Mrna expression. These regions are transcribed as polyadenylated segments in the untranslated portion of the Mma encoding tissue factor protein. The 3′ untranslated regions also include transcription termination sites. In some embodiments, the transcription unit also contains a polyadenylation region. One benefit of this region is that it increases the likelihood that the transcribed unit will be processed and transported like Mrna. The identification and use of polyadenylation signals in expression constructs is well established. In some embodiments, homologous polyadenylation signals be used in the transgene constructs. In one embodiment of the transcription unit, the polyadenylation region is derived from the SV40 early polyadenylation signal and consists of about 400 bases. The transcribed units may contain other standard sequences alone or in combination with the above sequences improve expression from, or stability of, the construct.

d. Markers

The viral vectors can include nucleic acid sequence encoding a marker product. This marker product is used to determine if the gene has been delivered to the cell and once delivered is being expressed. Non-limiting marker genes are the E. Coli lacZ gene which encodes β-galactosidase and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR⁻ cells and mouse LTK⁻ cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P. J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

The methods of the invention are useful for sensitive detection of one or multiple analytes. In general, the methods involve the use of special label components, referred to as reporter signals, that can be associated with, incorporated into, or otherwise linked to the analytes, or that can be used merely in conjunction with analytes, with no significant association between the analytes and reporter signals. In some embodiments of the methods, the reporter signals (or derivatives of the reporter signals) are detected, thus indicating the presence of the associated analytes. In other embodiments, the analyte (or derivatives of the analytes) are detected along with the reporter signals (or derivatives of the reporter signals).

In some embodiments, the disclosed methods involve two basic steps. A filtering, selection, or separation step to separate reporter signals (and/or the analytes attached thereto) from other molecules that may be present, and a detection step that distinguishes different reporter signals, different labeled analytes, or both. The reporter signals (and/or analytes attached thereto) may be are distinguished and/or separated from other molecules based on some common property shared by the reporter signals but not present in most (or, all) of the other molecules present. The labeled analytes can also be distinguished and/or separated from other molecules based on a common property of the labeled analyte as a whole, such as the mass-to-charge ratio of the labeled analyte. The separated reporter signals are then treated and/or detected such that the different reporter signals are distinguishable. Useful forms of the disclosed method involve association of reporter signals with analytes of interest. Detection of the reporter signals results in detection of the corresponding analytes. Thus, the disclosed method is a general technique for labeling and detection of analytes.

Optionally, the selection step can be preceded by fractionation step where a subset of analytes, including the analytes that are, or will be, labeled, are separated from other components in a sample. Such a step, although not necessary, can improve the selection step by reducing the number of extraneous molecules present.

The disclosed reporter signals also can be captured, sorted, immobilized, separated and the like using specific binding molecules that are specific for reporter signals and/or sets of reporter signals. Thus, for example, the common property can be used for the separation step or operation while binding of reporter signals to a specific binding molecule can be used to separate reporter signals (and molecules to which they are attached or bound) to be separated or sorted prior to use of the common property to separate components having the common property form other components. By using, for example, a specific binding molecule that can bind the reporter signals in a set of reporter signals (or all of the reporter signals being used in a given assay), the reporter signals can be separated from other components and materials that may be present. This can allow, for example, much cleaner detection and/or analysis of reporter signals.

A non-limiting form of the disclosed method involves filtering of isobaric reporter signals from other molecules based on mass-to-charge ratio, fragmentation of the reporter signals to produce fragments having different masses, and detection of the different fragments based on their mass-to-charge ratios. The different fragments will include the fragment of the reporter signal and the fragmented labeled analyte (made up of the analyte and the remaining part of the reporter signal). Either or both may be detected and will be characteristic of the initial labeled analyte.

The method is carried out, for example, using a tandem mass spectrometer where the isobaric reporter signals are passed through a filtering quadrupole, the reporter signals are fragmented in a collision cell, and the fragments are distinguished and detected in a time-of-flight (TOF) stage. In such an instrument the sample is ionized in the source (for example, in a MALDI ion source) to produce charged ions. In some embodiments, the ionization conditions are such that primarily a singly charged parent ion is produced. Sets of reporter signals (or analytes attached thereto, or reporter signal fusions) can also be filtered, separated and/or sorted by using antibodies or other specific binding molecules that can bind the reporter signal peptides.

The same sample can be analyzed both with and without fragmentation (by operating with and without collision gas), and the results compared to detect shifts in mass-to-charge ratio. Both the unfragmented and fragmented results should give diagnostic peaks, with the combination of peaks both with and without fragmentation confirming the reporter signal (and analyte) involved. Such distinctions are accomplished by using appropriate sets of isobaric reporter signals and allows large scale multiplexing in the detection of analytes.

The disclosed method is particularly well suited to the use of a MALDI-QqTOF mass spectrometer. The method enables highly multiplexed analyte detection, and very high sensitivity. Non-limiting tandem mass spectrometers are described by Loboda et al., Design and Performance of a MALDI-QqTOF Mass Spectrometer, in 47^{th} ASMS Conference, Dallas, Tex. (1999), Loboda et al., Rapid Comm. Mass Spectrom. 14(12):1047-1057 (2000), Shevchenko et al., Anal. Chem., 72: 2132-2142 (2000), and Krutchinsky et al., J. Am. Soc. Mass Spectrom., 11(6):493-504 (2000). In such an instrument the sample is ionized in the source (MALDI, for example) to produce charged ions; the ionization conditions are such that primarily a singly charged parent ion is produced. First and third quadrupoles, Q0 and Q2, will be operated in RF only mode and will act as ion guides for all charged particles, second quadrupole Q1 will be operated in RF+DC mode to pass only a particular mass-to-charge (or, in practice, a narrow mass-to-charge range). This quadrupole selects the mass-to-charge ratio, (m/z), of interest. The collision cell surrounding Q2 can be filled to appropriate pressure with a gas to fracture the input ions by collisionally induced dissociation (normally the collision gas is chemically inert, but reactive gases are contemplated).

A MALDI source is useful for the disclosed method because it facilitates the multiplexed analysis of samples from heterogeneous environments such as arrays, beads, microfabricated devices, tissue samples, and the like. An example of such an instrument is described by Qin et al., A practical ion trap mass spectrometer for the analysis of peptides by matrix-assisted laser desorption/ionization., Anal. Chem., 68:1784-1791 (1996). For homogeneous assays electrospray ionization (ESI) sources will work very well. Electrospray ionization source instruments interfaced to LC systems are commercially available (for example, QSTAR from PE-SCIEX, Q-TOF from Micromass). It is of note that the ESI sources are operated such that they tend to produce multiply charged ions, doubly charged ions would be most common for ions in the disclosed method. Such doubly charged ions are well known in the art and present no limitation to the disclosed method. TOF analyzers and quadrupole analyzers are useful detectors over sector analyzers. Tandem in time ion trap systems such as Fourier Transform Ion Cyclotron Resonance (FT-ICR) mass spectrometers also may be used with the disclosed method.

A number of elements contribute to the sensitivity of the disclosed method. The filter quadrupole, Q1, selects a narrow mass-to-charge ratio and discriminates against other mass-to-charge ions, significantly decreasing background from non germane ions. For example, for a sample containing a distribution of mass-to-charges of width 3000 Da, a mass-to-charge transmission window of 2 Da applied to this distribution can improve the signal to noise by at least a factor of 3000/2=1500. Once the parent ion is selected by quadrupole Q1, fragmentation of the parent ion, e.g., into a single charged daughter ion, has the advantage over systems which fragment the parent into a number of daughter ions. For example, a parent fragmented into 20 daughter ions will yield signals that are on average 1/20^{th} the intensity of the parent ions. For a parent to single daughter system there will not be this signal dilution.

This system for use with the disclosed method has a high duty cycle, and as such good statistics can be collected quickly. For the case where a single set of isobaric parents is used, the multiplexed detection is accomplished without having to scan the filter quadrupole (although such a scan is useful for single pass analysis of a complex protein sample with multiple labeled proteins). Electrospray sources can operate continuously, MALDI sources can operate at several kHz, quadrupoles operate continuously, and time of flight analyzers can capture the entire mass-to-charge region of interest at several kHz repetition rate. Thus, the overall system can acquire thousands of measurements per second. For throughput advantage in a multiplexed assay the time of flight analyzer has an advantage over a quadruple analyzer for the final stage because the time of flight analyzer detects all fragment ions in the same acquisition rather than requiring scanning (or stepping) over the ions with a quadrupole analyzer.

Instrumental improvements including addition of laser ports along the flight path to allow intersection of the proteins with additional laser(s) open additional fragmentation avenues through photochemical and photophysical processes (for example, selective bond cleavage, selective ionization). Use of lasers to fragment the proteins after the filter stage will enable the use of the very high throughput TOF-TOF instruments (50 kHz to 100 kHz systems).

The disclosed method is compatible with techniques involving cleavage, treatment, or fragmentation of a bulk sample in order to simplify the sample prior to introduction into the first stage of a multistage detection system. The disclosed method is also compatible with any desired sample, including raw extracts and fractionated samples.

In some embodiments, the method involves detection of labeled analytes in two or more samples in the same assay. This allows simple and consistent detection of differences between the analytes in the samples. Differential detection is accomplished by labeling the analytes in each sample with a different reporter signal. In some embodiments, the different reporter signals used for the different samples will make up an isobaric set. In this way, the same labeled analyte in each sample will have the same mass-to-charge ratio as that labeled analyte in a different sample. Upon fragmentation of the reporter signals, however, each of the fragmented labeled analytes in the different samples will have a different mass-to-charge ratio and thus each can be separately detected. All can be detected in the same measurement. This is a tremendous advantage in both time and quality of the data. For example, since the samples are assayed in a single run, there is no need to correct or normalize the results of different samples assayed in different runs. This allows accurate comparisons of the relative amounts of the same analyte in different samples since that are measured in the same run. There would be no differences to cause inconsistency between the samples.

A non-limiting use for this multiple sample mode of the disclosed method is the analysis of a time series of samples. Such series are useful for detecting changes in a sample or reaction over time. For example, changes in analyte levels in a cell culture over time after addition of a test compound can be assessed. In this mode, different time point samples are labeled with different reporter signals, e.g., making up an isobaric set. In this way, the same labeled analyte for each time point will have the same mass-to-charge ratio as that labeled analyte from a different time point. Upon fragmentation of the reporter signals, however, each of the fragmented labeled analytes from the different time points will have a different mass-to-charge ratio and thus each can be separately detected.

The disclosed method can also be used to gather and catalog information about unknown analytes. This analyte discovery mode can easily link the presence or pattern of analytes with their analysis. For example, a sample of labeled analytes can be compared to analytes in one or more other samples. Analytes that appear in one or some samples but not others can be analyzed using conventional techniques. The object analytes will be distinguishable from others by virtue of the disclosed labeling, detection, and quantitation. This mode of the disclosed method is especially useful as an aid to functional genomics or proteomics since proteins discovered to differ between samples can be characterized. This mode of the method is carried out, for example, using mass spectrometry.

In some embodiments, the disclosed method allows a complex sample of analytes to be quickly and easily cataloged in a reproducible manner. Such a catalog can be compared with other, similarly prepared catalogs of other analyte samples to allow convenient detection of differences between the samples. The catalogs, which incorporate a significant amount of information about the analyte samples, can serve as fingerprints of the samples which can be used both for detection of related analyte samples and comparison of analyte samples. For example, the presence or identity of specific organisms can be detected by producing a catalog of analytes of the test organism and comparing the resulting catalog with reference catalogs prepared from known organisms. Changes and differences in analyte patterns can also be detected by preparing catalogs of analytes from different cell samples and comparing the catalogs. Comparison of analyte catalogs produced with the disclosed method is facilitated by the fine resolution that can be provided with, for example, mass spectrometry.

Each labeled analyte processed in the disclosed method will result in a signal based on the characteristics of the labeled analyte (for example, the mass-to-charge ratio). A complex analyte sample can produce a unique pattern of signals. It is this pattern that can allow unique cataloging of analyte samples and sensitive and powerful comparisons of the patterns of signals produced from different analyte samples.

The presence, amount, presence and amount, or absence of different labeled analytes forms a pattern of signals that provides a signature or fingerprint of the analytes, and thus of the analyte sample based on the presence or absence of specific analytes or analyte fragments in the sample. For this reason, cataloging of this pattern of signals (that is, the pattern of the presence, amount, presence and amount, or absence of labeled analytes) is an embodiment of the disclosed method that is of particular interest.

Catalogs can be made up of, or be referred to, as, for example, a pattern of labeled analytes, a pattern of the presence of labeled analytes, a catalog of labeled analytes, or a catalog of analytes in a sample. The information in the catalog may be in the form of mass-to-charge information or compositional information. Catalogs can also contain or be made up of other information derived from the information generated in the disclosed method (for example, the identity of the analytes detected), and can be combined with information obtained or generated from any other source. The informational nature of catalogs produced using the disclosed method lends itself to combination and/or analysis using known bioinformatics systems and methods.

Such catalogs of analyte samples can be compared to a similar catalog derived from any other sample to detect similarities and differences in the samples (which is indicative of similarities and differences in the analytes in the samples). For example, a catalog of a first analyte sample can be compared to a catalog of a sample from the same type of organism as the first analyte sample, a sample from the same type of tissue as the first analyte sample, a sample from the same organism as the first analyte sample, a sample obtained from the same source but at time different from that of the first analyte sample, a sample from an organism different from that of the first analyte sample, a sample from a type of tissue different from that of the first analyte sample, a sample from a strain of organism different from that of the first analyte sample, a sample from a species of organism different from that of the first analyte sample, or a sample from a type of organism different from that of the first analyte sample.

The same type of tissue is tissue of the same type such as liver tissue, muscle tissue, or skin (which may be from the same or a different organism or type of organism). The same organism refers to the same individual, animal, or cell. For example, two samples taken from a patient are from the same organism. The same source is similar but broader, referring to samples from, for example, the same organism, the same tissue from the same organism, the same analyte, or the same analyte sample. Samples from the same source that are to be compared can be collected at different times (thus allowing for potential changes over time to be detected). This is especially useful when the effect of a treatment or change in condition is to be assessed. Samples from the same source that have undergone different treatments can also be collected and compared using the disclosed method. A different organism refers to a different individual organism, such as a different patient, a different individual animal. Different organism includes a different organism of the same type or organisms of different types. A different type of organism refers to organisms of different types such as a dog and cat, a human and a mouse, or E. coli and Salmonella. A different type of tissue refers to tissues of different types such as liver and kidney, or skin and brain. A different strain or species of organism refers to organisms differing in their species or strain designation as those terms are understood in the art.

When comparing catalogs of analytes obtained from related samples, it is possible to identify the presence of a subset of correlated pairs of labeled analytes and their altered forms. The disclosed method can be used to detect the original labeled analytes (and determine characteristics of them) and the altered form of the labeled analytes. This pair of detected analytes will be characteristic of the analyte that is labeled and the specific reporter signal used (although not necessarily unique).

S. Illustrations

The disclosed methods can be further understood by way of the following illustrations which involve examples of the disclosed methods. The illustrations are not intended to limit the scope of the method in any way.

a. Illustration 1: Heavy Isotopes

This illustration makes use of peptide reporter signals having the same mass, that fragment at certain peptide bonds, and that use heavy isotopes to distribute mass differently in different reporter signals. For example, it has been demonstrated, in ion traps, that peptides containing arginine will preferentially fragment at the C-termini of aspartic acid or glutamic acid residues, and, proline containing peptides will fragment at the N-termini of the proline residues (Qin and Chait, Int. J. Mass Spectrom. (Netherlands), 190-191:313-20 (1999)). DP (aspartic acid (D) and proline (P)) amino acid sequences can be used in the disclosed reporter signals resulting in collisionally induced fragmentation at the scissile bond between the aspartic acid and proline.

The singly charged ion of an exemplary peptide, AGSLDPAGSLR (SEQ ID NO:2), will fragment between the ‘D’ and ‘P’ in the collision cell of the mass spectrometer. Utilizing natural abundance isotopes the singly charged parent ion will have an average nominal (m/z)=1043 amu, and the possible resultant daughter ions AGSLD⁺ (amino acids 1-5 of SEQ ID NO:2) and PAGSLR⁺ (amino acids 6-11 of SEQ ID NO:2) have average nominal (m/z) of 461 and 600 amu, respectively. As a practical matter, fragmentation will typically yield one dominant daughter ion, say PAGSLR⁺ (amino acids 6-11 of SEQ ID NO:2) in this case. For this illustration consider only one charged daughter from the population of singly charged parent. Note that, without loss of generality or applicability, the branching ratio into these daughter ion channels may be other than 100% into the PAGSLR⁺ (amino acids 6-11 of SEQ ID NO:2) daughter fragment.

Standard synthetic methods can be utilized to construct such peptides. In this illustration of reporter molecules consider isotopically labeled amino acids (for example, A vs. A*, where A has a CH₃ and A* has a CD₃ side chain). There are four possibilities for the synthetic peptide, with their nominal (m/z) indicated in parentheses: AGSLDPAGSLR (1043), A*GSLDPAGSLR (1046), AGSLDPA*GSLR (1046), A*GSLDPA*GSLR (1049) (SEQ ID NO:2). For this example consider the two mono-labeled peptides A*GSLDPAGSLR, AGSLDPA*GSLR (SEQ ID NO:2), which have a common nominal mass-to-charge of 1046.

As a simple demonstration of a non-limiting mode of the disclosed method consider a solution containing the two synthetic peptides. This solution could have been collected following any number of biological experiments and, in general, because of processing, would contain many additional components.

The solution containing A*GSLDPAGSLR and AGSLDPA*GSLR (SEQ ID NO:2) is mixed with a suitable matrix solution for performing analysis by mass spectrometry. Suitable matrices, including sinapic acid, 4-hydroxy-α-cyanocinamic acid or 2,5-dihydroxybenzoic acid, are known in the art.

The resulting solution is spotted onto the MALDI target and allowed to crystallize.

The target is inserted into the source of the mass spectrometer. Utilizing the laser impinging on the sample spot on the MALDI target, many ions are introduced into the first quadrupole, Q0. Among the species introduced into Q0 are predominantly singly charged species (A*GSLDPAGSLR⁺, AGSLDPA*GSLR⁺; SEQ ID NO:2), various fragmentation ions, neutral matrix, matrix ions and multimers as known in the art. Neutral particles will pass out of Q0 without being guided into the second quadrupole, Q1.

Ions introduced into Q0 are guided into the higher vacuum region containing Q1.

Quadrupole Q1 is set to pass ions with the mass-to-charge ratio of 1046 into the third quadrupole, Q2 (recall A*GSLDPAGSLR and AGSLDPA*GSLR (SEQ ID NO:2) have the same mass-to-charge; “isobaric” in the parlance of mass spectrometry). Ions with mass-to-charge ratios different from 1046 will follow trajectories that do not exit Q1 on the Q1-Q2 axis, and are effectively discarded. This yields a huge increase in the signal to noise for the system, on the order of 100-1000 fold improvement over systems which do not have this mass filtering.

The collision cell surrounding Q2 is filled with a chemically inert gas at an appropriate pressure to cause preferential cleavage of the DP scissile bond of the peptide ions, typically a few milliTorr of nitrogen. As discussed above, the fragmentation of the singly charged parent ion is expected to yield predominantly one daughter ion. In this case each of the isobaric parents (SEQ ID NO:2) will yield correlated, unique daughters (amino acids 1-5 and 6-11 of SEQ ID NO:2):

A*GSLDPAGSLR⁺ → A*GSLD + PAGSLR⁺ (m/z 600)
AGSLDPA*GSLR⁺ → AGSLD + PA*GSLR⁺ (m/z 603)

The resolution of the mass spectrometers as discussed here is on the order of 5000 to 10000, and thus the 3 amu difference is readily attained at these (m/z).

The ions exiting Q2 enter the time-of-flight (TOF) section of the instrument. A transient electric field gradient is applied and the positively charged ions are accelerated toward the reflectron and ultimately to the detector. The ions are all accelerated through the same electric field gradient (the reflectron will compensate for a small perturbation in this assertion, as is known in the art) and thus will all have the same kinetic energy imparted to them. Because the kinetic energy is the same for all ions, and the masses of the ions are different, the time it takes for the ions to reach the detector will be different: heavier ions will arrive later than lighter ions.

The resulting mass spectrum reflects the relative amount of the two analytes (for example, peptides) in the original sample.

This scheme can be extended to more analytes (for example, peptides). The most basic extension for a panel of isobaric detectors based upon the above peptide, utilizing X/X* differences, would be as shown in Table 2. The asterisk indicates heavy isotope labeled amino acids. This set assumes that the non-labeled to labeled mass change {(m/z)_{X*}-(m/z)_{X}} for each residue is the same. For the general case where {(m/z)_{X*}-(m/z)_{X}} is not the same for all the residues there are more combinations for a given peptide which can be resolved by the mass spectrometer. The parent molecule is SEQ ID NO:2 and the primary daughter is amino acids 6-11 of SEQ ID NO:2.

TABLE 2

| | |
|---|---|
| Parent | Primary Daughter |

| | |
|---|---|
| A*G*S*L*DPAGSLR | PAGSLR |
| AG*S*L*DPA*GSLR | PA*GSLR |
| AGS*L*DPA*G*SLR | PA*G*SLR |
| AGSL*DPA*G*S*LR | PA*G*S*LR |
| AGSLDPA*G*S*L*R | PA*G*S*L*R |

The synthesis of specific isotope labeled amino acids would facilitate rapidly increased panel size. For example, synthesis of unique alanines with CH₃, CH₂D, CHD₂, CD₃ side chains could be used to yield a significant panel size with a small peptide.

This mode of the disclosed method has the desirable property that all the detected ions originate from a very similar chemical environment (only differing by the location of a few neutrons) and will thus behave identically (for all practical purposes) when processed in the MALDI source and in the collision cell. Of particular note is the case where one of the isobaric reporter signal molecules is added as a quantitation standard to the isobaric detector molecules used for the assay. Quantitation of the entire set of detector molecules used in the assay is straightforward and quantitative. For the case where the molecules are essentially identical except for the isotopic enrichment all the isobars in a set will behave identically through the processing.

b. Illustration 2: Labile Bond, One Daughter Ion

This illustration makes use of peptide reporter signals having the same mass that fragment at a labile bond, where the labile bond is placed in different locations in the different reporter signals. In this illustration, the parent ion produces a single daughter. An example of synthesis of peptides with labile bonds at defined positions between amino acids is disclosed by WO 97/11958. Analogous chemistry may be utilized to produce peptides with labile bonds between amino acids for use in the disclosed method and compositions. For example, consider a pair of peptide molecules of the form GSWFSGMCAR (SEQ ID NO:12):

| | |
|---|---|
| Peptide A: | GSWFSG#MCAR |
| Peptide B: | GSWF#SGMCAR |

where the symbol # indicates the location of the labile bond. Note that the peptide sequence does not have to be conserved for this method, the only requirement is that the molecular mass of the peptides be the same.

For simplicity consider a solution containing the two aforementioned synthetic peptides with labile bonds, A and B. This solution could have been collected following any number of biological experiments and, in general, because of processing, would contain many additional components.

The solution containing A and B is mixed with a suitable matrix solution for performing analysis by mass spectrometry. Suitable matrices, including sinapic acid, 4-hydroxy-α-cyanocinamic acid or 2,5-dihydroxybenzoic acid, are known in the art.

The resulting solution is spotted onto the MALDI target and allowed to crystallize.

The target is inserted into the source of the mass spectrometer.

Utilizing the laser impinging on the sample spot on the MALDI target, many ions are introduced into the first quadrupole, Q0. Among the species introduced into Q0 are predominantly singly charged species (A⁺, B⁺), various fragmentation ions, neutral matrix, matrix ions and multimers as known in the art. Neutral particles will pass out of Q0 without being guided into Q1.

Ions introduced into Q0 are guided into the higher vacuum region containing Q1.

Quadrupole Q1 is set to pass ions with the mass-to-charge ratio of (m/z)_{A} and (m/z)_{B} (recall (m/z)_{A}=(m/z)_{B}; “isobaric” in the parlance of mass spectrometry). Ions with mass-to-charge ratios different from (m/z)_{A} and (m/z)_{B} will follow trajectories that do not exit Q1 on the Q1-Q2 axis, and are effectively discarded. This yields a huge increase in the signal to noise for the system, on the order of 100-1000 fold improvement over systems which do not have this mass filtering.

The collision cell surrounding Q2 is filled with a chemically inert gas at an appropriate pressure to cause preferential cleavage of the labile bond of the peptide ions A⁺ and B⁺, typically a few milliTorr of nitrogen. Considering only fragmentation at the labile bond, and the operation of Q2 in RF only mode, there will be four possible ions which can emerge from Q2 into the TOF section. As discussed above, depending upon the thermodynamics and kinetics, it is common that one of the daughters for each parent will be more likely to take the charge than the other daughter. For the majority of cases there will be one predominant daughter ion. The primary fragmentation will be (SEQ ID NO:12 into amino acids 1-6 and 7-10 of SEQ ID NO:12 and SEQ ID NO:12 into amino acids 1-4 and 5-10 of SEQ ID NO:12):

GSWFSG#MCAR⁺ → GSWFSG + MCAR⁺
GSWF#SGMCAR⁺ → GSWF + SGMCAR⁺

The ions exiting Q2 enter the time-of-flight, TOF, section of the instrument. A transient electric field gradient is applied and the positively charged ions are accelerated toward the reflectron and ultimately to the detector. The ions are all accelerated through the same electric field gradient (the reflectron will compensate for a small perturbation in this assertion, as is known in the art) and thus will all have the same kinetic energy imparted to them. Because the kinetic energy is the same for all ions, and the masses of the ions are different, the time it takes for the ions to reach the detector will be different: heavier ions will arrive later than lighter ions.

The resulting mass spectrum shows the relative amount of the two reporter signals in the original sample.

A standard, with the same mass as the peptides (say GSW#FSGMCAR; SEQ ID NO:12), could have been added to facilitate quantitative results. In order to extract quantitative results the relative efficiencies of the isobaric detector molecule under consideration should be calibrated; a straightforward process.

c. Illustration 3: Labile Bond, Two Daughter Ions

This illustration makes use of peptide reporter signals having the same mass that fragment at a labile bond, where the labile bond is placed in different locations in the different reporter signals. In this illustration, the parent ion branches into two daughters. Consider the peptides as described in Illustration 2 (SEQ ID NO:12):

| | |
|---|---|
| Peptide A: | GSWFSG#MCAR |
| Peptide B: | GSWF#SGMCAR |

where the symbol # indicates the location of the labile bond. Note that the peptide sequence does not have to be conserved for this method, the only requirement is that the molecular mass of the reporter molecule peptides be nominally the same.

For simplicity consider a solution containing the two aforementioned synthetic peptides with labile bonds, A and B. This solution could have been collected following any number of biological experiments and, in general, because of processing, would contain many additional components.

The solution containing A and B is mixed with a suitable matrix solution for performing analysis by mass spectrometry. Suitable matrices, including sinapic acid, 4-hydroxy-α-cyanocinamic acid or 2,5-dihydroxybenzoic acid, are known in the art.

The resulting solution is spotted onto the MALDI target and allowed to crystallize.

The target is inserted into the source of the mass spectrometer.

Utilizing the laser impinging on the sample spot on the MALDI target, many ions are introduced into the first quadrupole, Q0. Among the species introduced into Q0 are predominantly singly charged species (A⁺, B⁺), various fragmentation ions, neutral matrix, matrix ions and multimers as known in the art. Neutral particles will pass out of Q0 without being guided into Q1.

Ions introduced into Q0 are guided into the higher vacuum region containing Q1.

Quadrupole Q1 is set to pass ions with the mass-to-charge ratio of (m/z)_{A} and (m/z)_{B} (recall (m/z)_{A}=(m/z)_{B}; “isobaric” in the parlance of mass spectrometry). Ions with mass-to-charge ratios different from (m/z)_{A} and (m/z)_{B} will follow trajectories that do not exit Q1 on the Q1-Q2 axis, and are effectively discarded. This yields a huge increase in the signal to noise for the system, on the order of 100-1000 fold improvement over systems which do not have this mass filtering.

The collision cell surrounding Q2 is filled with a chemically inert gas at an appropriate pressure to cause preferential cleavage of the labile bond of the peptide ions A⁺ and B⁺, typically a few milliTorr of nitrogen. Considering only fragmentation at the labile bond, and the operation of Q2 in RF only mode, there will be four possible ions which can emerge from Q2 into the TOF section. As discussed above for the majority of cases there will be a predominant daughter ion. The fragmentation of the population of singly charged parent ions into the daughter may be as follows (these branching ratios would be empirically determined) (SEQ ID NO:12 into amino acids 1-6 and 7-10 of SEQ ID NO:12 and SEQ ID NO:12 into amino acids 1-4 and 5-10 of SEQ ID NO:12):

```
GSWFSG#MCAR⁺ → GSWFSG + MCAR⁺ (A1: 50%)
                          → GSWFSG⁺ + MCAR (A2: 50%)
GSWF#SGMCAR⁺ → GSWF + SGMCAR⁺ (B1: 50%)
                          → GSWF⁺ + SGMCAR (B2: 50%)
```

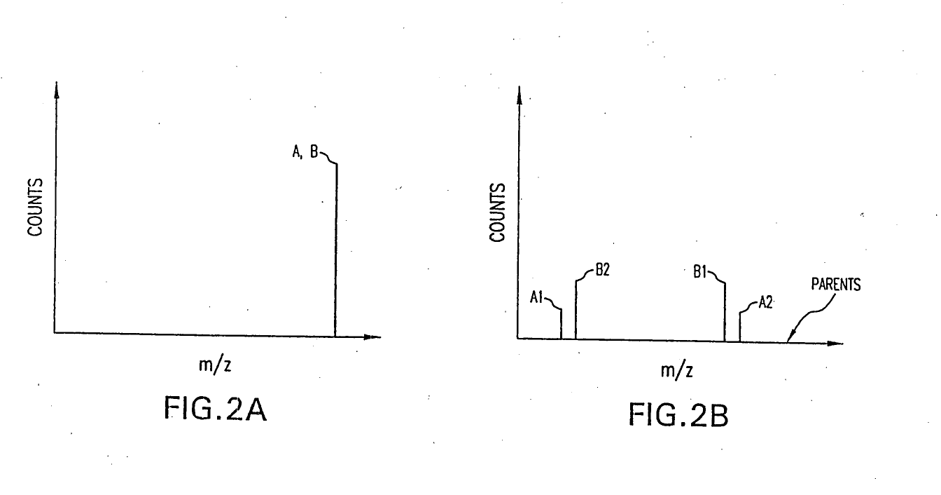

The branching ratios as noted here would yield a mass spectrum as shown schematically in FIG. 2. The spectrum indicates there is twice as much B as A in the original sample. In the case of very low pressure in the collision cell the parent ions will pass through Q2 without fragmenting (FIG. 2A), with gas in the collision cell the peptides will fragment at the labile bonds (FIG. 2B). Note the correlation (intensities are the same, and the sum of the masses is equal to the parent ion mass-to-charge) of the A⁺ daughters and the B⁺ daughters.

The ions exiting Q2 enter the time-of-flight, TOF, section of the instrument. A transient electric field gradient is applied and the positively charged ions are accelerated toward the reflectron and ultimately to the detector. The ions are all accelerated through the same electric field gradient (the reflectron will compensate for a small perturbation in this assertion, as is known in the art) and thus will all have the same kinetic energy imparted to them. Because the kinetic energy is the same for all ions, and the masses of the ions are different, the time it takes for the ions to reach the detector will be different: heavier ions will arrive later than lighter ions.

The resulting mass spectrum shows the relative amount of the two analytes (for example, peptides) in the original sample. The daughter ion signals will be correlated with each other at known branching ratio and known parent ion (m/z), and thus there is increased confidence in the measurement of the analytes.

A standard, with the same mass as the analytes (say GSW#FSGMCAR; SEQ ID NO:12), could have been added to facilitate quantitative results. In order to extract quantitative results the relative efficiencies of the isobars under consideration should be calibrated.

d. Illustration 4: Scissile Bond

This illustration makes use of peptide reporter signals having the same mass that fragment at certain peptide bonds, where the bond is placed in different locations in the different reporter signals. As discussed above, DP containing amino acid sequence will fragment between the aspartic acid and proline in a collision cell. A set of peptides that may be useful for the disclosed method may be:

| | | |
|---|---|---|
| Peptide C: | YFMTSGCDPGGR | (SEQ ID NO:13) |
| Peptide D: | YFMTSGDPCGGR | (SEQ ID NO:14) |
| Peptide E: | YFMTSDPGCGGR | (SEQ ID NO:15) |
| Peptide F: | YFMTDPSGCGGR | (SEQ ID NO:16) |
| Peptide G: | YFMDPTSGCGGR | (SEQ ID NO:17) |

For simplicity consider a solution containing these synthetic peptides. This solution could have been collected following any number of biological experiments and, in general, because of processing would contain many additional components.

The solution containing C, D, E, F, G is mixed with a suitable matrix solution for performing analysis by mass spectrometry. Suitable matrices, including sinapic acid, 4-hydroxy-α-cyanocinamic acid or 2,5-dihydroxybenzoic acid, are known in the art.

The resulting solution is spotted onto the MALDI target and allowed to crystallize.

The target is inserted into the source of the mass spectrometer.

Utilizing the laser impinging on the spot on the MALDI target, many ions are introduced into the first quadrupole, Q0. Among the species introduced into Q0 are C⁺, D⁺, E⁺, F⁺, G⁺, various fragmentation ions, matrix ions and multimers as known in the art. Neutral particles will pass out of Q0 without being guided into Q1.

Ions introduced into Q0 are guided into the higher vacuum region containing Q1.

Quadrupole Q1 is set to pass ions with the mass-to-charge ratio of (m/z)_{C}, (m/z)_{D}, (m/z)_{E}, (m/z)_{F}, (m/z)_{G} (they have the same molecular weight “isobaric”). Ions with mass-to-charge ratios different from (m/z)_{C}, (m/z)_{D}, (m/z)_{E}, (m/z)_{F}, (m/z)_{G} will follow trajectories which will not exit Q1 on the Q1-Q2 axis, and are effectively discarded. This yields a huge increase in the signal to noise for the system, on the order of 100-1000 fold improvement over systems which do not have this mass filtering.

The collision cell surrounding Q2 is filled with a chemically inert gas at an appropriate pressure to cause scission of the D-P bond, typically a few milliTorr of nitrogen. Considering only fragmentation at the DP bond, and total retention of the charge by the C termini fragments, and the operation of Q2 in RF only mode, there will be five possible ions which can emerge from Q2 into the TOF section.

| | | |
|---|---|---|
| G1⁺: | PGGR⁺ | (amino acids 9-12 of SEQ ID NO:13) |
| D1⁺: | PCGGR⁺ | (amino acids 8-12 of SEQ ID NO:14) |
| E1⁺: | PGCGGR⁺ | (amino acids 7-12 of SEQ ID NO:15) |
| F1⁺: | PSGCGGR⁺ | (amino acids 6-12 of SEQ ID NO:16) |
| G1⁺: | PTSGCGGR⁺ | (amino acids 5-12 of SEQ ID NO:17) |

The ions exiting Q2 enter the time-of-flight, TOF, section of the instrument. A transient electric field gradient is applied and the positively charged ions are accelerated toward the reflectron and ultimately to the detector. The ions are all accelerated through the same electric field gradient (the reflectron will compensate for a small perturbation in this assertion, as is known in the art) and thus will all have the same kinetic energy imparted to them. Because the kinetic energy is the same for all ions, and the masses of the ions are different, the time it takes for the ions to reach the detector will be different: heavier ions will arrive later than light ions.

The resulting mass spectrum will indicate the relative amount of the analytes (for example, peptides) in the original sample.

A standard with the same mass as the analytes could have been added to facilitate quantitative results. In order to extract quantitative results the relative efficiencies of molecules under consideration should be determined to be used in calibration; a straightforward process.

e. Illustration 5: Pre Treatment Direct Readout.

This illustration involves release of the reporter signal from a specific binding molecule prior to the first quadrupole of the instrument. The specific binding molecule may be a DNA, a PNA, an antibody, or any other moiety with high specificity and affinity. The reporter signal is attached to the specific binding molecule through an interaction which can be selectively broken through the use of, for example, restriction enzymes, photocleavable nucleotides (WO 00/04036), photocleavable linkages (Olejnik et al., Nucleic Acids Res., 27(23):4626-31 (1999)), and biotin-advidin like interactions (Niemeyer et al., Nucleic Acids Res., 22(25):5530-9 (1994), Sano et al., Science, 258(5079):120-2 (1992)).

An exemplary set of constructs might have the general form Nⱼ-Xₖ, where the nucleotides are indicated by N and are PNA, the amino acids are indicated by X, the dash indicates the transition from PNA to peptide through a photocleavable linkage, and ‘j’ and ‘k’ are independent integers. Two members of such an exemplary set are (SEQ ID NO:18; peptide portion):

| | |
|---|---|
| H: | ACGGCGACGTGGCTAATC-A*G*S*L*A*G*S*L*DPAGSLAGSLR |
| I: | CGAGAGCTAGCTATATGC-AG*S*L*A*G*S*L*DPA*GSLAGSLR |

where the asterisk indicates a heavy amino acid as described in Illustration 1. The PNA will direct specific molecular recognition such that ‘H’ will recognize GATTAGCCACGTCGCCGT (SEQ ID NO:19) and ‘I’ will recognize GCATATAGCTAGCTCTCG (SEQ ID NO:20). Processing in an analogous fashion to the above illustrations, the photocleavable linkage will be broken by the MALDI laser pulse and the peptide isobar signal molecules will be selected by the Q1 mass filter, and one will detect PAGSLAGSLR⁺ and PA*GSLAGSLR⁺ (amino acids 10 to 19 of SEQ ID NO:18) for ‘H’ and ‘I’ reporter molecules respectively.

Design of DNA-peptide constructs where an internal restriction site is engineered into the DNA strand would enable a DNA specific binding molecule and a peptide reporter signal. Endonucleases Hha I, HinP1 I and Mnl I are known to have significant single strand activity (NEB catalog). A prototypical reporter molecule, utilizing Hha I (GCGˆC), could have the form (SEQ ID NO:21, DNA portion; SEQ ID NO:18, peptide portion)

GACGACGGCGACGTGGCTGCGC-A*G*S*L*A*G*S*L*DPAGSLAGSLR

where GACGACGGCGACGTGGCT (nucleotides 1 to 18 of SEQ ID NO:21) represents the specific binding molecule, GCGC is the recognition site for Hha I, and the dash represents the transition from DNA to peptide. For this mode of the disclosed method, the set of molecules would all share the underlined sequence adjacent to the transition to the peptide. Pretreatment with Hha I will cleave the all molecules containing GCGC leaving the 3′ cytosine nucleotide attached to the peptide.

In an analogous manner, because one has freedom over the peptide sequence, one can make use of the huge body of literature in the art for specific cleavage of peptides to specifically cleave the reporter molecule within the peptide moiety. Examples of such specific cleavage systems include thrombin (cleaves between Arg and Gly), trypsin (cleaves C-terminus of Arg or Lys), endoprotease Glu-C (cleavages C-terminus of Asp or Glu), and the general classes known as oligopeptidases or endoproteases.

f. Illustration 6: Indirect Readout

In a preferred embodiment of the disclosed method, a reporter molecule containing a decoding tag is used to specifically recognize a coding molecule. As an example consider a coding molecule which has the recognition sequence as shown for ‘H’ in Illustration 5 (SEQ ID NO:22 and SEQ ID NO:23)

ACGGCGACGTGGCTAATC-spacer-CGTCATCGTAG

where the specific binding molecule will recognize and associate with GATTAGCCACGTCGCCGT (SEQ ID NO:19), -spacer- is a convenient spacer moiety such as PEG, and, CGTCATCGTAG (SEQ ID NO:23) represents a coding tag. The reporter molecule is of the form Nⱼ-Xₖ, where the nucleotides are indicated by N and are PNA, the amino acids are indicated by X, the dash indicates the transition from PNA to peptide (optionally through a cleavable linkage), and ‘j’ and ‘k’ are independent integers. An example is (SEQ ID NO:18, peptide portion)

CTACGATGACG-A*G*S*L*A*G*S*L*DPAGSLAGSLR

The PNA, which is the decoding tag, will recognize and specifically associate with the CGTCATCGTAG (SEQ ID NO:23) coding tag of the coding molecule

(SEQ ID NO:22 and SEQ ID NO:23)

```
ACGGCGACGTGGCTAATC-spacer-CGTCATCGTAG
                                                    GCAGTAGCATC
                                                    |
```

(SEQ ID NO:18)

```
                                                    A*G*S*L*A*G*S*L*
                                                    DPAGSLAGSLR
```

During processing as described above, the reporter molecule ion may be selected by the filter quadrupole, Q1, and read out through the daughter fragments. In the optional case where the link between the PNA and the peptide may be selectively broken the filter quadrupole, Q1, would be tuned to the mass-to-charge of the peptide ion.

A set of molecules for multiplex assay only requires the reporter molecule to have a common mass among the set (or a common mass among the set of peptides, in the case of the selective bond breakage between the PNA and the peptide). A common mass for the reporter molecule is easily attained simply by utilizing alternate sequence of the PNA preserving the composition of the PNA (that is, same number of A, C, G, T residues in all instances) in combination with the peptide isobar detector molecules as described in Illustration 1.

A clear advantage of this mode of the disclosed method is the ability to separately optimize the specific binding molecule and the reporter signal of the reporter molecules. A minor constraint on the coding tag of the coding molecule is that among a set the A, C, G, T content must remain fixed.

g. Illustration 7: Detection of SH2 and SH3 Domains in Proteins of a Single Sample

This illustration involves detection of individual SH2 and SH3 domains in particular proteins. The SH2 and SH3 domains of proteins are of considerable interest in the field of proteomics, and of particular relevance in the field of oncology. Consider a sample containing two known proteins that each contain both the SH2 and SH3 domains: human c-src and v-src. A capture moiety that recognizes these domains (such as an antibody) can be used to select proteins containing these domains from a sample. A pair of such protein sequences is shown below.

c-src (NCBI reference GI:11433119; SEQ ID NO:9)
MSAIQAAWPS CTECIAKYNF HGTAEQDLPF CKGDVLTIVA VTKDPNWYKA KNKVGREGII
PANYVQKREG VKAGTKLSLM PWFHGKITRE QAERLLYPPE TGLFLVREST NYPGDYTLCV
SCDGKVEHYR IMYHASKLSI DEEVYFENLM QLVEHYTSDA DGLCTRLIKP KVMEGTVAAQ
DEFYRSGWAL NNKELKLLQT IGKGEFGDVM LGDYRGNKVA VKCIKNDATA QAFLAEASVM
TQLRHSNLVQ LLGVIVEEKG GLYIVTEYMA KGSLVDYLRS RGRSVLGGDC LLKFSLDVCE
AMEYLEGNNF VHRDLAARNV LVSEDNVAKV SDFGLTKEAS TPRTRASCQS SGQPLRP
v-src (NCBI reference GI:11421794; SEQ ID NO:10)
MGSNKSKPKD ASQRRRSLEP AENVHGAGGG AFPASQTPSK PASADGHRGP SAAFAPAAAE
PKLFGGFNSS DTVTSPQRAG PLAGGVTTFV ALYDYESRTE TDLSFKKGER LQIVNNTEGD
WWLAHSLSTG QTGYIPSNYV APSDSIQAEE WYFGKITRRE SERLLLNAEN PRGTFLVRES
ETTKGAYCLS VSDFDNAKGL NVKHYKIRKL DSGGFYITSR TQFNSLQQLV AYYSKHADGL
CHRLTTVCPT SKPQTQGLAK DAWEIPRESL RLEVKLGQGC FGEVWMGTWN CTTRVAIKTL
KPGTMSPEAF LQEAQVMKKL RHEKLVQLYA VVSEEPIYIV TEYMSKGSLL DFLKGETGKY
LRLPQLVDMA AQIASGMAYV ERMNYVHRDL RAANILVGEN LVCKVADFGL ARLIEDNEYT
ARQGAKFPIK WTAPEAALYG RFTIKSDVWS FGILLTELTT KGRVPYPGMV NREVLDQVER
GYRMPCPPEC PESLHDLMCQ CWRKEPEERP TFEYLQAFLE DYFTSTEPQY QPGENL

The SH3 and SH2 domains are indicated in double underline and single underline respectively. Cysteine residues are indicated in bold. These can be labeled by covalent sulfur-sulfur bridges. Tryptic digest of the c-src and v-src proteins results in the fragments shown in Table 3.

Consider a reporter signal of composition CGAGSDPLAGSLR (m/z=1203; SEQ ID NO:11) and labeling of the cysteine residues of the c-src and v-src proteins with this reporter signal through formation of covalent bond between the sulfur groups of the cysteine of the protein and the sulfur groups of cysteine of the reporter signal. Table 3 shows the masses of the unlabeled, labeled and altered fragments.

In this illustration, the reporter signals are peptides that have been designed to have a preferred fragmentation site. Peptides containing arginine will preferentially fragment at the C-termini of aspartic acid or glutamic acid residues, and, proline containing peptides will fragment at the N-termini of the proline residues (Qin and Chait, Int. J. Mass Spectrom. (Netherlands), 190-191:313-20 (1999)). Thus, DP (aspartic acid (D) and proline (P)) amino acid sequences are used in the reporter signals resulting in collisionally induced fragmentation at the scissile bond between the aspartic acid and proline.

| | | | | |
|---|---|---|---|---|
| | | | | Labeled |
| | | | Labeled | PLAGSLR |
| | | Unlabeled | mass | fragment |
| Source | Fragment | mass (amu) | (amu) | loss (amu) |

| | | | | |
|---|---|---|---|---|
| c-src, SH3 domain | MSAIQAAWPSGTECIAK | 1763 | 2964 | 2252 |
| c-src, SH3 domain | YNFHGTAEQDLPFCK | 1769 | 2972 | 2260 |
| c-src, SH2 domain | ESTNYPGDYTLCVSCDGK | 1951 | 4353 | 2929 |
| c-src, SH2 domain | LSIDEEVYFENLMQLVEHYTSDADGLCTR | 389 | 1590 | 878 |
| c-src | CIK | 362 | 1563 | 851 |
| c-src | SVLGGDCLLK | 1003 | 2504 | 1792 |
| c-src | FSLDVCEAMEYLEGNNFVHR | 2372 | 2573 | 1861 |
| c-src | ASCQSSGQPLR | 1230 | 2731 | 2019 |
| v-src, SH2 domain | GAYCLSVSDFDNAK | 1489 | 2690 | 1978 |
| v-src, SH2 domain | HADGLCHR | 907 | 2108 | 1396 |
| v-src | LTTVCPTSKPQTQGLAK | 1772 | 2973 | 2261 |
| v-src | LGQGCFGEVWMGTWNGTTR 2099 | 3300 | 2588 | |
| v-src | AANILVGENLVCK | 1343 | 2544 | 1832 |
| v-src | MPCPPECPESLHDLMCQCWR | 2374 | 7178 | 4330 |

Table 3. Fragments resulting from tryptic digest of CGAGSDPLAGSLR (SEQ ID NO:11) labeled src proteins. The fragments are, from top to bottom, amino acids 1-17 of SEQ ID NO:9, amino acids 18-32 of SEQ ID NO:9, amino acids 108-125 of SEQ ID NO:9, amino acids 138-166 of SEQ ID NO:9, amino acids 223-225 of SEQ ID NO:9, amino acids 284-293 of SEQ ID NO:9, amino acids 294-313 of SEQ ID NO:9, amino acids 346-357 of SEQ ID NO:9, amino acids 185-198 of SEQ ID NO:10, amino acids 236-243 of SEQ ID NO: 10, amino acids 244-260 of SEQ ID NO: 10, amino acids 276-294 of SEQ ID NO: 10, amino acids 392-404 of SEQ ID NO:10, amino acids 484-503 of SEQ ID NO:10. Also shown is the resulting mass of the labeled fragment after loss of the PLAGSLR fragment (amino acids 7-13 of SEQ ID NO:11).

Tryptic digests of the proteins are introduced into a mass spectrometer. Ions corresponding to the masses in the labeled mass column of Table 3 are selected and fragmented in the collision cell, subsequently analyzed in the TOF. The collision energy and collision gas density are tuned such that the primary fragmentation is the scissile bond between aspartic acid (D) and proline (P).

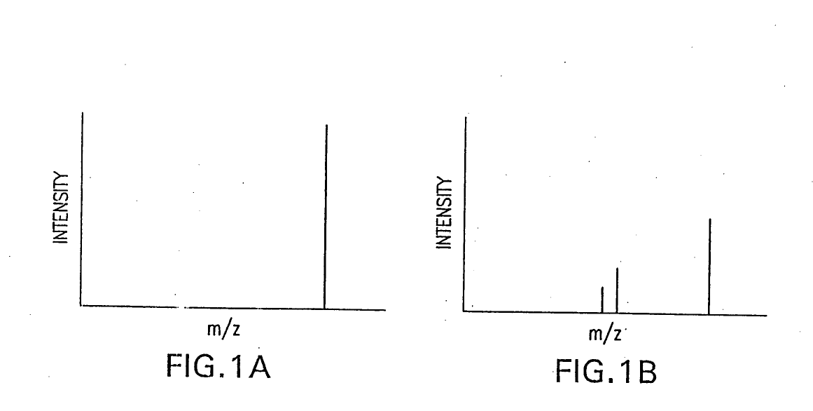

The existence of reporter signals are clearly seen by the parent ion mass-to-charge shift of a multiple of the PLAGSLR (amino acids 7-13 of SEQ ID NO:11) units upon fragmentation at the scissile bonds, or in some cases (as determined by the molecular dissociation kinetics, dynamics and thermodynamics) a PLAGSLR⁺ (amino acids 7-13 of SEQ ID NO:11) will be directly observed. For example, the labeled protein C(CGAGSDPLAGSLR)IK (amino acids 223-225 of SEQ ID NO:9 and SEQ ID NO:11), shown in row 5 in Table 3, would be selected in the first quadrupole at 1563 amu, and would fragment to yield 851 amu (and possibly 712 amu for PLAGSLR⁺; amino acids 7-13 of SEQ ID NO:11). In contrast, unlabeled fragments of the same nominal mass (there are none in this illustration) would be selected by the first quadrupole but would not exhibit the 712 amu shift nor the 712 amu peak. This yields an exceptional discrimination against unlabelled fragments. A representation of the mass spectrum is shown in FIG. 1.

For an unknown fragment, or to confirm a known fragment, determined to contain the label, the sequence can be obtained using standard MS/MS peptide sequencing techniques without further processing.

This illustration demonstrates a simple case of detection of a pair of known proteins using the method (via detection of particular fragments of the proteins). This method is extensible to a large number of proteins, known and/or unknown, in a complex mixture. The combination event of the parent signal and the fragmentation ion(s) provides an enormous discrimination against the “background”.

If further fractionation is desired automated industrial systems, such as HPLC or capillary electrophoresis, may be inserted in front of the mass spectrometer to increase the discrimination further. Fractionation systems may be used in tandem arrangement (for example, LC/LC). In the fields of protein discovery and functional genomics, biological fractionation may be employed using interactions of interest, for example a functionally related system such as an affinity partner for the SH2 and SH3 domains to capture the families.

h. Illustration 8: Protein Profiling of SH2 and SH3 Domains in Proteins of a Multiple Samples

Consider the protein c-src as described in Illustration 7 and its tryptic fragments as described in Table 3.

The temporal protein expression of c-src produced by a stimulus applied to stable Jurkat T cells (see, for example, Brdicka et al., Phosphoprotein associated with glycosphingolipid-enriched microdomains (PAG), a novel ubiquitously expressed transmembrane adaptor protein, associates with the protein tyrosine kinase csk and is involved in regulation of T cell activation. J Exp Med., 191:1591-604 (2000)) can be followed by collecting sample cells at defined time following the stimulus and lysing the cells. The SH2 and SH3 domain containing proteins (including c-src) may be captured at this point in the procedure. Each lysate is then labeled with a different reporter signal from Table 1 and the proteins are digested with trypsin.

Consider the specific example of the c-src tryptic fragment CIK shown in row 5 in Table 3. For fixed times of 0, 1, 2, 3 and 4 hours the lysates are labeled with CG*G*G*G*DPGGGGR, CG*G*G*GDPGGGG*R, CG*G*GGDPGGG*G*R, CG*GGGDPGG*G*G*R, and GGGGDPG*G*G*G*R (SEQ ID NO:1), respectively that will yield PGGGGR⁺, PGGGG*R⁺, PGGG*G*R⁺, PGG*G*G*R⁺, and PG*G*G*G*R⁺ (amino acids 7 to 12 of SEQ ID NO:1) respectively upon collisional fragmentation. Five time point measurements are obtained in a single measurement by introducing the labeled protein mixture into the mass spectrometer, setting the first filter to pass the isobaric set of labeled proteins (at mass-to-charge corresponding to C(CG*G*G*G*DPGGGGR)IK; SEQ ID NO:1), fragmenting the reporter signal and measuring the reporter signals at m/z=499, 500, 501, 502, 503 in order to detect fragments having the characteristic mass-to-charge ratio for each of the time points (0, 1, 2, 3 and 4 hours, respectively).

Other labeled proteins of interest are selected with the filter and quantitated in similar fashion.

i. Illustration 9: Protein Fragment Detection with Reporter Signal Calibrators

This illustration involves detection of protein fragments using reporter signal calibrators.

1. A suspension containing 1000 cells is centrifuged to get a cell pellet.

2. The cells are lysed using detergent.

3. The lysate is digested with trypsin.

4. Optionally, the protein digest is oxidized with hydrogen peroxide or derivatized with acetylacetone.

5. The material placed in a tandem mass spectrometer, ionized, selected by a mass-to-charge filter, fragmented, mass analyzed, and detected, in order to measure the signals from unique fragile tryptic peptides and the corresponding reporter signal calibrator standard designed for each unique tryptic peptide.

6. Mass spectrometry detection is repeated with different, specific filtering settings for 50 different peptide mass/charge ratios suitable for each signature tryptic peptide and its corresponding reporter signal calibrator peptide.

7. Data is collected as a catalog of 50×2 independent measurements, constituting the peptide signature.

j. Illustration 10: Reporter Signal Fusions, Expressed From Plasmid Vectors, With Epitope Tags

This illustration provides an example of, and an example of the use of, a set of simple expression vectors encoding an amino acid segment that includes an epitope tag that is the same in all the vectors, and that includes a reporter signal peptide that is different in all the vectors of the set of vectors. The reporter signal peptides can be cleaved from the amino acid segment with trypsin. All of the reporter signal peptides have the same mass-to-charge ratio, but, when fragmented, produce fragments that have different mass-to-charge ratios.

1. A set of different DNA plasmid vectors is constructed containing the following elements:

(a) a common origin of replication and a common antibiotic selectable marker,

(b) an inducible promoter (for this illustration, the promoter could be the same for all plasmids or different for each plasmid), and

(c) a nucleic acid segment encoding an amino acid segment (the reporter signal fusion) where the amino acid segment includes:
(1) a protein of interest to be expressed under the control of (that is, operably linked to) the promoter (for this illustration, the protein could be different for each plasmid or could be the same for all plasmids),
(2) a common epitope tag, such as a flag peptide, and
(3) a reporter signal peptide that can be released from the amino acid segment upon trypsin digestion (for this illustration, each plasmid encodes a different reporter signal peptide where each reporter signal peptide belongs to the same isobaric set of reporter signal peptides).

2. Each plasmid vector is introduced individually into transformation-competent cells.

3. Transformed cells are grown under the antibiotic selection.

4. The inducible promoter is induced by its appropriate activator compound.

5. The expressed amino acid segments (that is, reporter signal fusions) are measured as follows:

(a) the cells, harboring different expression vectors, are mixed in a single vessel,

(b) the mixture of cells is lysed to release all proteins,

(c) an antibody specific for the epitope tag is used to purify (separate) the reporter signal fusion(s) from the lysate,

(d) the epitope tag-purified reporter signal fusion is digested with trypsin,

(e) the tryptic peptides are combined with matrix and analyzed by MALDI-tandem mass spectrometry where the amount of each different reporter signal is measured.

k. Illustration 11: Reporter Signal Fusions, Expressed From Plasmid Vectors, With Cis-Cleavable Linkage

This illustration provides an example of, and an example of the use of, a set of expression vectors encoding reporter signal fusions with a peptide-release mechanism based on activatable self-cleavage proteolytic activity of an intein, or any suitable cis-acting protease. The proteolytic activity serves to control the release of the reporter signal peptide present in the each of the reporter signal fusions.

1. A set of different DNA plasmid vectors is constructed harboring the following sequence elements:

(a) an origin of replication and an antibiotic selectable marker,

(b) an inducible promoter, wherein the promoter may be the same for all plasmids, or different for each plasmid,

(c) a nucleic acid segment encoding an amino acid segment (the reporter signal fusion), to be expressed under the direction of the promoter, where the amino acid segment includes:
(1) a protein of interest, wherein the protein could be different for each plasmid, or could be the same for all plasmids,
(2) an intein protein domain located such as to be able to catalyze release of the reporter signal peptide by a cis-cleavage reaction.
(2) a reporter signal peptide belonging to a specific isobaric set of reporter signal peptides, wherein each plasmid encodes a different member of the isobaric set of reporter signal peptides.

2. The plasmid vector is introduced into transformation-competent cells.

3. Transformed cells are grown under the antibiotic selection.

4. The inducible promoter is induced by its appropriate activator compound.

5. The expressed reporter signal fusion is measured as follows:

(a) the cells are lysed to release internal proteins,

(b) DTT is added to activate the intein self-cleavage activity (Chong et al. (1998) Utilizing the C-terminal cleavage activity of a protein splicing element to purify recombinant proteins in a single chromatographic step. Nucleic Acids Res 26:5109-5115),

(c) the released peptides are combined with matrix and analyzed by MALDI-tandem mass spectrometry where the amount of each different reporter signal is measured.

l. Illustration 12: Reporter Signal Fusions, Expressed From BAC Vectors, With Epitope Tags

This illustration provides an example of, and an example of the use of, a set of mammalian BAC expression vectors with recombinase sites capable of driving integration in specific gene loci.

1. A set of different BAC vectors derived from pEYMT (Hong et al. (2001) Development of two bacterial artificial chromosome shuttle vectors for a recombination-based cloning and regulated expression of large genes in mammalian cells. Analytical Biochemistry 291:142-148) is constructed. These vectors are capable of shuttling between bacteria, yeast and mammalian cells. The vectors have the following features:

(a) a common promoter,

(b) a nucleic acid segment encoding an amino acid segment (the reporter signal fusion), to be expressed under the direction of the promoter, where the amino acid segment includes:
(1) one of a set of proteins of interest, wherein the protein coding sequence is different for each BAC, or, alternatively, the protein coding sequence is the same for all BACs, but the BACs then are made different by the use of a different promoter in each BAC,
(2) an epitope tag, such as the flag peptide,
(3) a reporter signal peptide belonging to a specific isobaric set of reporter signal peptides, whereby the reporter signal fusion is tagged with the epitope tag and a unique reporter signal peptide, whereby the reporter signal peptide may be released by trypsin digestion.

2. Each of the BAC vectors is introduced individually into mouse embryonic stem cells, to achieve integration in genomic DNA. The transformed ES cells are introduced into an embryo, to generate a chimeric animal, containing ES cells in the germline. The progeny of these mice are screened to identify transgenic mice that harbor the integrated reporter signal fusion construct.

3. Tissue is obtained from each transgenic animal, and equal amounts of tissue from several animals is mixed.

4. The mixture of tissues is lysed to release proteins.

5. An antibody specific for the epitope tag (for example, anti-flag antibody) is used to purify the reporter signal fusions.

6. The flag-purified proteins are digested with trypsin.

7. The tryptic peptides are combined with matrix and analyzed by MALDI-tandem mass spectrometry where the amount of each different reporter signal is measured.

m. Illustration 13: Reporter Signal Fusions, Expressed From Plant Vectors, With Epitope Tags and Recombinase Sites

This illustration provides an example of, and an example of the use of, a set of plant expression vectors with two directly oriented lox site sites capable of driving integration in a specific recipient gene locus (slightly modified from Vergunst et al. (1998) Site-specific integration of Agrobacterium T-DNA in Arabidopsis thaliana mediated by Cre recombinase. Nucleic Acids Res 26:2729-2734).

1. A set of different Agrobacterium T-DNA vectors is constructed harboring the following sequence elements:

(a) A Floxed T-DNA recombination cassette, without a promoter (Vergunst et al. (1998) Site-specific integration of Agrobacterium T-DNA in Arabidopsis thaliana mediated by Cre recombinase. Nucleic Acids Res 26:2729-2734), designed to be integrated in the genome of a recipient plant by Cre recombinase-driven integration, with the cassette comprising a nucleic acid segment encoding an amino acid segment (the reporter signal fusion), to be expressed under the direction of the promoter, where the amino acid segment includes:

(1) a coding sequence for a protein of interest, wherein the protein could be different for each T-DNA, or could be the same for all T-DNAs,

(2) an epitope tag, such as the flag peptide,

(3) a reporter signal peptide belonging to a specific isobaric set of reporter signal peptides.

2. The T-DNA plasmid vector is introduced into recipient plants, such plants harboring a chimeric promoter-lox-Cre gene, under the control of a chemically inducible promoter (Kunkel et al. (1999) Inducible isopentenyl transferase as a high-efficiency marker for plant transformation. Nature Biotechnology 17:916-919), designed to receive the recombinant protein cassette of the integrative vector by Cre-driven recombination. As in the original design of Vergunst and co-workers (1998), site-specific integration simultaneously leads to loss of Cre-expression, making the insertion event irreversible.

3. The expressed, integrated reporter signal fusion is generated under the direction of the chemically inducible promoter present in front of the integrated gene. Expression is measured as follows:

(a) tissue is obtained from each plant, and equal amounts of tissue from several plants is mixed,

(b) the mixture of tissues is lysed to release proteins,

(c) an antibody specific for the epitope tag (i.e., anti-flag) is used to purify the reporter signal fusions,

(d) the flag-purified proteins are digested with trypsin,

(e) the tryptic peptides are combined with matrix and analyzed by MALDI-tandem mass spectrometry where the amount of each different reporter signal is measured.

n. Illustration 14: Kit Including Vectors Encoding Reporter Signal Fusions

This illustration provides an example of a kit comprising a set of 2 or more vectors encoding reporter signal fusions. The kit comprises two or more expression vectors, wherein each vector expresses a different reporter signal fusion, wherein all reporter signal peptides in the reporter signal fusions belong to single isobaric set. The kit also can contain reagents needed for use of the vectors, such as

(a) Reagents for transformation,

(b) Reagents for inducing release of reporter signal peptides from reporter signal fusions, and

(c) Reagents for performing mass spectral analysis, such as a matrix optimized for analysis of reporter signal peptides.

o. Illustration 15: Reporter Signal Fusions Encoded by PCR Products

This illustration provides an example of, and an example of the use of, a set of reporter signal fusions designed for expression in a rabbit reticulocyte cell-free system, where the DNA encoding the reporter signal fusion is generated by PCR.

1. PCR primers are designed to amplify a protein sequence of interest, whereby the one of the PCR primers contains a T7 RNA polymerase promoter, and a Kozak translational initiation sequence, positioned correctly in relation to the AUG start codon. The PCR primers also contain sequences coding for a reporter signal peptide, which may be placed at the amino terminus or at the carboxyl terminus of the protein of interest (thus forming a reporter signal fusion). Each reporter signal peptide is designed such as to cleavable from the protein by trypsin digestion.

2. The artificial gene is amplified by PCR, to generate sufficient DNA.

3. The DNA generated by PCR is transcribed in vitro using T7 RNA polymerase.

4. The solution containing the transcribed DNA is added to a rabbit reticulocyte in vitro translation system, to generate the reporter signal fusion product.

5. The in vitro synthesized reporter signal fusion is used with or without purification.

6. The process is repeated for other variants of the protein of interest. In a typical application, as many as 128 different protein variants may be generated by in vitro transcription/translation.

p. Illustration 16: Reporter Signal Fusions Encoded by PCR Products

This illustration provides an example of, and an example of the use of, a set of reporter signal fusions encoding by nucleic acid molecules designed for expression in an E. coli coupled transcription/translation system.

1. PCR primers are designed to amplify a protein sequence of interest, whereby the one of the PCR primers contains a T7 RNA polymerase promoter, and a Shine-Dalgarno translational initiation sequence, positioned correctly in relation to the AUG start codon. The PCR primers also contain sequences coding for a reporter signal peptide, which may be placed at the amino terminus or at the carboxyl terminus of the protein of interest (thus forming a reporter signal fusion). Each reporter signal peptide is designed such as to cleavable from the protein by trypsin digestion.

2. The artificial gene is amplified by PCR, to generate sufficient DNA for use in a coupled in vitro transcription/translation system.

3. The DNA generated by PCR is incubated in the in vitro coupled transcription/translation system, to generate the reporter signal fusion product.

4. The in vitro synthesized reporter signal fusion is used with or without purification.

5. The process is repeated for other variants of the protein of interest. In a typical application, as many as 128 different protein variants may be generated by in vitro transcription/translation.

q. Illustration 17: Reporter Signal Fusions Expressed in Yeast Cells

This illustration provides an example of, and an example of the use of, a set of 32 yeast strains, each strain harboring a single reporter signal fusion.

A yeast strain (Saccharomyces cerevisiae) is constructed, using homologous recombination targeted to a non-essential gene, whereby a fusion of a candidate therapeutic protein and a reporter signal peptide (belonging to a set of 32 isobaric reporter signal peptides) is placed under the control of a galactose-responsive promoter. Another 31 similar yeast strains are constructed, using the same yeast promoter, whereby the only other difference in the DNA sequence coding for the reporter signal fusion is the use of codons designed to generate one of 31 different reporter signal peptides, completing a set of 32 different promoters and an isobaric set of 32 distinct reporter signal peptides. The yeast strains may be used for any assay where the reporter signal peptides (and/or reporter signal fusions) serve as reporters for the expression of the protein fused to the reporter signal peptide, which in this case is a candidate therapeutic protein.

r. Illustration 18: Reporter Signal Fusions Expressed in Mouse Cells

This illustration provides an example of, and an example of the use of, a set of 32 mouse cell lines, each cell line harboring a single reporter signal fusion.

A mouse cell line is constructed, using an SV40 vector system, whereby a fusion of a candidate therapeutic protein and a reporter signal peptide (belonging to a set of 32 isobaric reporter signal peptides) is placed under the control of a cytokine promoter. Another 31 similar cell lines are constructed, using 31 different cytokine promoters, whereby the only other difference in the DNA sequence coding for the reporter signal fusion is the use of codons designed to generate one of 31 different reporter signal peptides, completing a set of 32 different promoters and an isobaric set of 32 distinct reporter signal peptides. The cell lines may be used for any assay where the reporter signal peptides (and/or reporter signal fusions) serve as reporters for the expression of the protein fused to the reporter signal peptide, which in this case is a candidate therapeutic protein.

s. Illustration 19: Reporter Signal Fusions Expressed Using Promoter of Interest

This illustration provides an example of the use of cells that harbor single fusions, as part of a cytokine-STAT5a-responsive promoter. This system can provide a comparison of reporter signal peptides versus a GFP internal standard.

1. An experiment is performed using a set of 96 different reporter signal peptides belonging to a unique mass set (that is, an isobaric set), where each cell line harbors a nucleic acid construct encoding a single reporter signal fusion. The fusion construct contains a cytokine-responsive promoter for the CIS1 protein, which is activated through a STATS response (Masumoto et al. (1999) Suppression of STAT5 functions in liver, mammary glands, and T cells in cytokine-inducible, SH2-containing protein 1 transgenic mice. Mol Cell Biol 19:6396-6407), a GFP-encoding sequence and a sequence encoding a reporter signal peptide fused to the GFP.

2. After treatment of 38,400 cell cultures for 6 hours with a set of 38,400 different cytokine-mimic drug candidates from a combinatorial drug library, a subset of 3,840 cell cultures are sampled to obtain values for GFP fluorescence.

3. The cells are pooled in groups of 96.

4. The mixture of cells is lysed to release the GFP fusion proteins.

5. The lysate is digested with trypsin.

6. The tryptic peptides are combined with matrix and analyzed by MALDI-tandem mass spectrometry where the amount of each different reporter signal is measured.

The readout speed of an expensive mass spectrometer is often the rate-limiting factor in proteomic analysis. A key feature of this method is the ability to pool sets of 96 treated cell samples prior to immunoprofiling based on mass spectrometry of reporter signal peptides. A total of 38,400 cell cultures are treated, each with a different drug. The use of pooling different cells harboring one of 96 different reporter signal peptides permits the 38,400 cultures to be analyzed as 400 pooled samples.

The GFP fluorescence values obtained in step 2 for a subset of the samples (3,840 out of 38,400) are compared to the data obtained by mass spectrometry analysis of the reporter signal peptides from the same samples. A good correlation between the fluorescence values and the values obtained by mass spectrometry constitutes a control for the function and utility of an reporter signal fusion-tagged cell line.

t. Illustration 20: Multiple Reporter Signal Fusions Expressed in a Cell

This illustration provides an example of a method for expression profiling of 32 different reporter signal fusions, utilizing cells that harbor multiple reporter signal fusions.

An experiment is performed using a set of 32 different reporter signal peptides belonging to a unique mass set (that is, they are isobaric), where each cell line harbors 32 different reporter signal fusions that are expressed independently, under the control of different promoters. Monitoring the expression of these 32 different proteins serves as a measure of drug toxicity.

Cell cultures are analyzed one at a time:

(a) the cells are lysed to release proteins,

(b) the lysate is digested with trypsin.

(c) the tryptic peptides are combined with matrix and analyzed by MALDI-tandem mass spectrometry where the amount of each different reporter signal is measured.

u. Illustration 21: Multiple Reporter Signal Fusions Expressed in a Mammalian Cell Line

This illustration provides an example of, and an example of the use of, a mammalian cell line, designed for use as a microencapsulated producer for heterologous protein delivery, the cell line harboring 12 reporter signal fusions.

A mammalian cell line is constructed, using a BAC homologous recombination vector system (Hong et al. (2001) Development of two bacterial artificial chromosome shuttle vectors for a recombination-based cloning and regulated expression of large genes in mammalian cells. Analytical Biochemistry 291:142-148), whereby a fusion of two candidate therapeutic proteins and two isobaric reporter signal peptides (belonging to a set of 12 isobaric reporter signal peptides) is placed under the control of a tetracycline promoter. Another 10 fusions in 10 genes, coding for different secretory proteins, are constructed in the same cell line, under the control of their own native promoters, whereby the only other difference in the DNA sequence coding for the fusion peptides is the use of codons designed to generate one of 10 different reporter signal peptides. This completes a set of 12 different genes and 12 distinct reporter signal peptides.

The cell line is microencapsulated, and used for heterologous protein delivery in an animal host, or a human patient, where secretion of the therapeutic proteins is induced by tetracycline.

An immunoassay is performed, where specific antibodies are used to capture the 12 reporter signal fusions of interest, whereby the reporter signal peptides (and/or reporter signal fusions) serve as reporters for the expression of each protein fused to one reporter signal peptide, including the two candidate therapeutic proteins. One may thus measure the response of the microencapsulated cells to tetracycline induction, and, simultaneously, the production of other secretory proteins by the microencapsulated cells.

This precise monitoring of protein expression by the microencapsulated cells, using reporter signal fusions, serves to accelerate the dosage optimization, and results in increased therapeutic safety control. Among the additional secretory proteins monitored by the method one may include cytokines or other proteins with mitogenic potential.

v. Illustration 22: Multiple Reporter Signal Fusions Expressed in Multiple Cell Lines

This illustration provides an example of, and an example of the use of, a set of six different human cell lines, each cell line harboring ten different reporter signal fusions, whereby all reporter signal peptides belong to the same isobaric set.

Six cell lines are derived from adult stem cells, where each cell line is representative of a different major human haplotype, defined by unique SNP combinations, whereby each of the six haplotypes is representative of an important pharmacogenomic drug response subset of the human population for beta(2)-adrenergic receptor (Drysdale et al. (2000) Complex promoter and coding region beta 2-adrenergic receptor haplotypes alter receptor expression and predict in vivo responsiveness. Proc Natl Acad Sci USA. 97:10483-10488).

A total of six mammalian cell lines are constructed, using a BAC homologous recombination vector system (Hong et al. (2001) Development of two bacterial artificial chromosome shuttle vectors for a recombination-based cloning and regulated expression of large genes in mammalian cells. Analytical Biochemistry 291:142-148), whereby a set of ten fusions in ten different genes, coding for different signal transduction proteins, are constructed in same cell line, under the control of their own native promoters, whereby the only other difference in the DNA sequence coding for the fusion peptides is the use of codons designed to generate only one of ten different reporter signal peptides. This completes a set of ten different genes and ten corresponding, distinct reporter signal fusions for each cell line, whereby each cell line represents a major haplotype of the human beta(2)-adrenergic receptor, and thus sixty different reporter signal fusions are present in the combined set of all six cell lines.

The cell lines may be used for any assay where the set of sixty reporter signal peptides serve as reporters for the expression of the specific proteins fused to each reporter signal peptide in each cell line.

w. Illustration 23: Multiple Reporter Signal Fusions Expressed in Multiple Human Cell Lines

This illustration provides an example of, and an example of the use of, a set of six cell lines, where each cell line is representative of a human haplotype, and each cell line harbors multiple reporter signal fusions.

1. An experiment is performed using a set of sixty different reporter signal peptides belonging to a unique mass set (that is, they are isobaric), where each cell line harbors ten reporter signal fusions. The objective of the experiment is to measure the response of the cells to a drug.

2. The cells are pooled in groups of six haplotypes.

3. The mixture of cells is lysed to release proteins.

4. The lysate is digested with trypsin.

5. The tryptic peptides are combined with matrix and analyzed by MALDI-tandem mass spectrometry where the amount of each different reporter signal is measured.

The readout speed of an expensive mass spectrometer is often the rate-limiting factor in proteomic analysis. A key feature of this method is the ability to pool sets of six treated cell samples prior to immunoprofiling based on mass spectrometry of reporter signal peptides. A total of 2,400 cell cultures are treated, each with a different drug. The use of pooling of six different cell lines permits the 2,400 cultures to be analyzed as 400 pooled samples. All 400 samples are deposited on the plate of a mass spectrometer, and analyzed by tandem mass spectrometry. The information for each laser shot consists of the expression levels of sixty different reporter signal fusions.

x. Illustration 24: Kit of Reporter Signal Fusion-Labeled Human Cell Lines

This illustration provides an example of, and an example of the use of, a kit comprising six cell lines, where each cell line is representative of a major human haplotype, and each cell line harbors multiple reporter signal fusions. The kit includes the cell lines, and a set of reporter signal peptide controls designed to be used in experiments that are performed using a set of sixty different reporter signal peptides belonging to a unique mass set, where each cell line harbors ten reporter signal fusions. The objective of the experiment is to the response of the cells to different drugs.

y. Illustration 25: Reporter Signal Fusion-Labeled Flies

This illustration provides an example of, and an example of the use of, a transgenic fruit fly harboring reporter signal fusions.

A recombinant fly of the species Drosophila melanogaster is constructed, using homologous recombination (Rong & Golic (2001) A targeted gene knockout in Drosophila. Genetics 157:1307-1312), so that a total of 16 genes are modified by addition of sequence encoding reporter signal fusions belonging to a unique mass set (that is, they are isobaric). The 16 recombinant proteins are chosen on the basis of their known function at various levels of different signal transduction pathways, such as ras, myc, etc. The fusion is located at either the carboxyl-terminus or the amino-terminus of each of the proteins, and may optionally be preceded by an epitope tag, such as the flag epitope.

The flies are used for an experiment in which a new genotype is generated by transformation with P-elements harboring members of a recombinant protein library. The objective of performing the transformation is to observe the phenotypes generated by different protein sequences present in the recombinant library.

After transformation, individual flies are processed to extract proteins, the proteins are digested with trypsin, and the reporter signal peptides derived from reporter signal fusions are analyzed by desorption-ionization using a nanostructured silicon film (Hayes et al. (2001) Desorption-ionization mass spectrometry using deposited nanostructured silicon films. Anal. Chem. 73:1292-1295), coupled with collision-induced fragmentation tandem mass spectrometric analysis. The reporter signal peptide profile generates a representation of the relative abundance of the reporter signal fusions in the fly.

z. Illustration 26: Reporter Signal Fusion-Labeled Mice

This illustration provides an example of, and an example of the use of, transgenic mice harboring reporter signal fusions for signal transduction pathway analysis.

A recombinant mouse of the species Mus musculus is constructed, using homologous recombination in embryonic stem (ES) cells, (Templeton et al. (1997) Efficient gene targeting in mouse embryonic stem cells. Gene Therapy 4:700-709), so that a total of 12 genes are modified by addition of reporter signal fusions belonging to a unique mass set (that is, they are isobaric). The gene fusions are designed by adding the reporter signal peptide at either the amino terminus or the carboxyl-terminus of each of the recombinant proteins of interest. The 12 recombinant proteins are chosen on the basis of their known key functions at various levels of different signal transduction pathways, such as ras, myc, wnt, etc. Some of the fusions may optionally contain an epitope tag, such as the flag epitope, or a GFP fusion. Some of the fusions may involve mouse proteins of unknown function.

Most of the recombinant reporter signal fusions are placed under their normal mouse promoter, while one or a few of the recombinant reporter signal fusions may be under the control of a heterologous promoter, to test a certain experimental hypothesis. For example, an experimental recombinant reporter signal fusion may consist of an interleukin-6 coding sequence, fused to an reporter signal peptide, under the (inappropriate) control of the interleukin-2 promoter.

Mice with normal promoters, as well as the mice with an experimental heterologous promoters linked to reporter signal fusions, are used in a series of experiment in which tumors are induced by a chemical mutagen (2-azoxymethane). After tumors appear, the mice are treated with different candidate anti-tumor drugs.

At different times after drug treatment, tumors are dissected from individual mice, and the tumor tissue is processed to extract proteins. The proteins are digested with trypsin, and the reporter signal peptides derived from reporter signal fusions are analyzed by desorption-ionization using a nanostructured silicon film (Hayes et al. (2001) Desorption-ionization mass spectrometry using deposited nanostructured silicon films. Anal. Chem. 73:1292-1295), coupled with collision-induced fragmentation tandem mass spectrometric analysis. The reporter signal peptide profile generates a representation of the relative abundance of the 12 reporter signal fusions, and this profile serves as an informative measure of multiple pathway responses to the antitumor drug in a normal mouse, or in a mouse with experimental heterologous promoter constructs. The profiles may also provide information regarding the expression of proteins of unknown function.

aa. Illustration 27: Reporter Signal Fusion-Labeled Mice

This illustration provides an example of, and an example of the use of, transgenic mice harboring reporter signal fusions for studying inflammatory responses and Cyclooxygenase 2 (Cox-2) promoter mutants.

A recombinant mouse of the species Mus musculus is constructed, using homologous recombination in embryonic stem (ES) cells, (Templeton et al. (1997) Efficient gene targeting in mouse embryonic stem cells. Gene Therapy 4:700-709), so that a total of ten genes are modified by addition of reporter signal fusions belonging to a unique mass set. The gene fusions are designed by adding the reporter signal peptide at either the amino terminus or the carboxyl-terminus of each of the recombinant proteins of interest. The ten recombinant proteins are chosen on the basis of their known function at various levels of tissue inflammatory responses (such as Cox-2, etc). Some of the fusions may optionally contain an epitope tag, such as the flag epitope, or a GFP fusion. Some of the fusions may involve mouse proteins of unknown function, but which are suspected to have a role in inflammation.

Most of the recombinant reporter signal fusions are placed under their normal mouse promoter, while one or a few of the recombinant reporter signal fusions may be under the control of a mutant promoter, to test a certain experimental hypothesis. For example, an experimental recombinant reporter signal fusion may consist of a Cox-2 coding sequence, fused to an reporter signal peptide, under the control of a reduced transcriptional response Cox-2 mutant promoter.

Mice with normal promoters, as well as the mice with an experimental mutant promoters linked to reporter signal fusions, are used in a series of experiments in which colonic inflammation and colitis is induced by Dextran sulfate, an then the mice are treated with anti-inflammatory drugs.

At different times after drug treatment, colons are dissected from individual mice, and the tissue is processed to extract proteins. The proteins are digested with trypsin, and the reporter signal peptides derived from reporter signal fusions are analyzed by desorption-ionization using a nanostructured silicon film (Hayes et al. (2001) Desorption-ionization mass spectrometry using deposited nanostructured silicon films. Anal. Chem. 73:1292-1295), coupled with collision-induced fragmentation tandem mass spectrometric analysis. The reporter signal peptide profile generates a representation of the relative abundance of the ten reporter signal fusions, and this profile serves as an informative measure of multiple pathway responses to the anti-inflammatory drug in the colon of a normal mouse with colitis, or in a mouse with experimental mutant Cox-2 promoter constructs. The profiles may also provide information regarding the expression of proteins of unknown function.

bb. Illustration 28: Multiple Samples Labeled With Different Reporter Signals

This illustration is an example of multiple sample labeling using reporter signals where each sample is labeled with a different reporter signal.

This illustration involves the use of 384 antibody mini-columns, in order to generate a profile of 384 different protein antigens. Antibodies are covalently coupled to agarose beads using standard water-soluble carbodiimide chemistry. (March et al. (1974) A simplified method for cyanogen bromide activation of agarose for affinity chromatography. Anal Biochem. 60:149-152). The capacity of a small (75 microliter) affinity column with one antibody is equal to approximately 2×10¹¹ molecules of each protein. If multiplexing of reporter signals is 256×, the maximum protein binding capacity will be approximately 10⁸ molecules. The dynamic range of the assay will thus be 10,000 to 100,000,000 molecules of protein.

1. Prepare 4 sets of 64 reporter signals (for a total of 256), each of the four sets having the same mass.

2. Label each of 256 cell preps by covalent coupling of a unique reporter signal, using standard heterobifunctional reagents such as SATA and SSCP (Pierce Chemicals). Preferred chemistry for this purpose is the use of Sulfo-LS-SPDP (cat #21650 from Pierce; Uto et al., Determination of urinary Tamm-Horsfall protein by ELISA using a maleimide method for enzyme-antibody conjugation, J. Immunol. Methods, 138:87-94 (1991).

3. Associate with affinity column on microtip containing specific antibody.

4. Repeat for 384 antibodies. That is, associate all of the labeled cell preps to each of the 384 columns.

5. Elute from column using photocleavable reporter signal-release-chemistry (Innovachem, Tucson, Ariz.; see, for example, Harth-fritschy and Cantacuzene, Pept. Res. 50:415 (1997)).

6. The reporter signals are combined with matrix and analyzed by MALDI-tandem mass spectrometry where the amount of each different reporter signal is measured, using 4 successive mass-to-charge settings (4×64), one for the mass of each of the four sets.

The number of data points will be 256×384=98,304. Ten runs per day would provide 980,000 data points. This method is easily scalable to 384×10 antibodies=3840 antibodies. For 3840 antibodies, 10 runs per day would give 9,830,400 data points per day.

cc. Illustration 29: Multiple Samples Labeled With Different Reporter Signals

This illustration is an example of multiple sample labeling using reporter signals where each sample is labeled with a different reporter signal. The samples are labeled via a DNA coding tag intermediate.

This illustration involves the use of 384 antibody mini-columns, in order to generate a profile of 384 different protein antigens. Antibodies are covalently coupled to agarose beads using standard water-soluble carbodiimide chemistry. (March et al. (1974) A simplified method for cyanogen bromide activation of agarose for affinity chromatography. Anal Biochem. 60:149-152). The capacity of a small (75 microliter) affinity column with one antibody is equal to approximately 2×10¹¹ molecules of each protein. If multiplexing of reporter signals is 256×, the maximum protein binding capacity will be approximately 10⁸ molecules. The dynamic range of the assay will thus be 10,000 to 100,000,000 molecules of protein.

Each of the protein preparations is tagged with a unique DNA oligonucleotides (coding tags), wherein a set of 64 different coding tags has the property of not being able to hybridize with each other. The protein preparation is reacted with 2-iminothiolane (Alagon and King, (1980) Activation of polysaccharides with 2-iminothiolane and its uses. Biochemistry. 19:4341-4345) to introduce reactive sulfhydryl groups, if none is present. A DNA oligonucleotide (the coding tag), containing a reactive amino group at one of its termini is reacted with a heterobifunctional cross-linking reagent, such as SULFO-SMCC (Pierce, Inc.). The thiol-containing proteins are incubated together with the activated oligonucleotide, to form a covalent protein-DNA adduct (thus labeling the protein with a coding tag). For most protein molecules, the formation of this covalent adduct will not interfere with the capacity of the protein to associate with its cognate antibody. A total of 64 protein preparations, each harboring covalently coupled unique coding tag sequences, are pooled together before being used for the multiplexed assay.

This example also involves the use of reporter molecules composed of peptide nucleic acid decoding tags and reporter signal peptides. The decoding tags comprise 64 different PNA sequences designed to be incapable of hybridizing to each other and additionally designed to be complementary to each of the 64 aforementioned coding tags used for protein labeling. The length of the PNA portion of the reporter molecule is preferably 9 to 15 bases, and more preferably 10 to 11 bases. The reporter molecules of this example also comprises 64 different sequences of amino acids (the reporter signal peptides) which have the common property of having the same mass, but being cleavable in such a way that they can be separated from each other after collision-induced fragmentation.

1. Label each of 64 cell preps with a unique, non-self hybridizing, DNA oligonucleotide (coding tag), using SULFO-SMCC chemistry as indicated above.

2. Associate with affinity column on microtip containing specific antibody.

3. Repeat for 384 antibodies. That is, associate all of the labeled cell preps to each of the 384 columns.

4. Pass all 64 reporter molecules through columns, to achieve hybridization of PNA to DNA coding tags on proteins.

5. Elute proteins from column using acid matrix.

6. Separate and quantify reporter signals by MALDI-tandem mass spectrometry where the amount of each different reporter signal is measured.

This example can be performed using peptide nucleic acid reporter signals (that is, reporter signals composed of peptide nucleic acid) to associate directly with the coding tags. The reporter signals would comprise 64 different PNA sequences of the same mass, designed to be incapable of hybridizing to each other, and additionally designed to be complementary to each of the 64 aforementioned coding tags used for protein labeling. The reporter signals could be easily dissociated from the coding tags (for detection) since they are only non-covalently associated with the coding tags.

dd. Illustration 30: Multiple Samples Labeled With Different Reporter Signals

This illustration is an example of multiple sample labeling using reporter signals where each sample is labeled with a different reporter signal. The samples are labeled via a DNA coding tag intermediate and the samples are analyzed using an antibody array.

This illustration involves the use of an antibody microarray of 3200 elements, constructed on a solid surface, the surface being compatible with analysis by mass spectrometry.

Each of the protein preparations is tagged with a unique DNA oligonucleotides (coding tags), wherein a set of 16 different oligonucleotides has the property of not being able to hybridize with each other. The protein preparation is reacted with 2-iminothiolane (Alagon and King, (1980) Activation of polysaccharides with 2-iminothiolane and its uses. Biochemistry. 19:4341-4345) to introduce reactive sulfhydryl groups, if none is present. A DNA oligonucleotide (coding tag), containing a reactive amino group at one of its termini is reacted with a heterobifunctional cross-linking reagent, such as SULFO-SMCC (Pierce, Inc.). The thiol-containing proteins are incubated together with the activated oligonucleotide, to form a covalent protein-DNA adduct, thus labeling the proteins with the coding tags. For most protein molecules, the formation of this covalent adduct will not interfere with the capacity of the protein to associate with its cognate antibody. A total of 16 protein preparations, each harboring covalently coupled unique DNA coding tag sequences, are pooled together before being used for the multiplexed assay.

As in Illustration 29, this example also involves the use of PNA-peptide reporter signals reporter molecules. The PNA portions are decoding tags and comprises 16 different PNA sequences, designed to be incapable of hybridizing to each other, and additionally designed to be complementary to sequences in each of the 16 aforementioned DNA tags used for protein labeling. The reporter signal portion of the reporter molecules comprises 16 different sequences of amino acids which have the common property of having the same mass, but being cleavable in such a way that they can be separated from each other after collision-induced fragmentation.

An additional property of the 16 coding tags used for tagging each of the 16 protein samples is that each coding tag is able to associate with eight molecules of the reporter molecule. Each tagged protein in the sample will contain, on the average, one to three DNA coding tags. Thus, each protein will be able to associate with many (8 to 24) reporter molecules. This design results in increased signal intensity of reporter signals in the mass spectrometer.

1. Label each of 16 cell preps with a unique, non-self hybridizing, DNA oligonucleotide (coding tag), using SULFO-SMCC chemistry as indicated above.

2. Place the sample on a microarray containing 3200 immobilized antibodies, the microarray being constructed on the surface of a plate suitable for reading on a mass spectrometer. Incubate for 2 hours at 37° C. Wash the surface to remove un-associated sample.

3. Contact the antibody microarray with a mixture of 16 PNA-peptide reporter signal reporter molecules. Wash to remove excess reporter molecules.

4. Coat the surface with matrix, and load the microarray into a MALDI tandem mass spectrometer.

5. Separate and quantify reporter signals by MALDI-tandem mass spectrometry where the amount of each different reporter signal is measured.

This example can be performed using peptide nucleic acid reporter signals (that is, reporter signals composed of peptide nucleic acid) to associate directly with the coding tags. The reporter signals would comprise 16 different PNA sequences of the same mass, designed to be incapable of hybridizing to each other, and additionally designed to be complementary to each of the 16 aforementioned coding tags used for protein labeling. The reporter signals could be easily dissociated from the coding tags (for detection) since they are only non-covalently associated with the coding tags.

### I. EXAMPLES

### a. Example 1

### Use of Isobaric Reporter Signals

An important property of some of the disclosed reporter signals is their use in sets where the reporter signals all have a common property (allowing the reporter signals to be separated from the “junk” based upon this common property) and where the reporter signals can be subsequently altered to allow the detection of the individual members of the set of reporter signals. A number of peptides were synthesized with particular sequences and compositions in order to demonstrate the manipulation and analysis of reporter signals utilizing a tandem mass spectrometer. For this example, a set of reporter signals of common mass but differing sequence was used. The reporter signals were fragmented to reveal a part of the sequence, and the reporter signal fragments were detected. Use of reporter signals having a scissile, -DP-, bond was demonstrated.

The quantification of multiple proteins from a complex mixture has not been adequately performed in the field of proteomics. Singly charged peptides containing a C-terminal arginine in an ion trap will preferentially fragment at the C-termini of aspartic acid or glutamic acid residues, and proline containing peptides will fragment at the N-termini of the proline residues (Qin and Chait, Collision-induced dissociation of singly charged peptide ions in a matrix-assisted laser desorption ionization ion trap mass spectrometer. Int. J. Mass Spectrom. (Netherlands), 190-191:313-20 (1999)). These principles were used in designing an exemplary set of peptide reporter signals making use of a DP amino acid sequence to test the collisional fragmentation at the scissile bond between the aspartic acid and proline.

A Micromass Q-TOF instrument (Micromass Inc., Beverly, Mass.) was used in this example. Peptides for this example were synthesized by Fmoc amino acid synthesis on a Rainin Symphony. The reaction scale was 25 μmol. Crude synthesis products were used in this example. The disclosed method should tolerate dirty samples and complex mixtures.

i. Initial Peptides

Eight peptides that varied in the amino acid sequence and/or incorporated isotopes of specific amino acids were synthesized. These were the reporter signals. Modified amino acids were used to demonstrate differential distribution of mass by differential distribution of heavy isotope in reporter signals (heavy isotope mode), and reporter signals of differing sequence were used to demonstrate differential distribution of mass by differential distribution of individual amino acids in reporter signals (variable sequence mode). Modified protected amino acids containing heavy stable isotopes were obtained from Cambridge Isotopes. The two amino acids used here were 3-¹³C-Ala and 2-¹³C-Gly which are each one Dalton heavier than their natural amino acids. Fmoc protected phosphorylated serine was used to further demonstrate the heavy isotope mode and also demonstrate the use of side chain modified amino acids. These peptides were synthesized with free NH₂ and free COOH on N and C termini, respectively, as shown in Table 4.

TABLE 4

| | | | Mode |
|---|---|---|---|
| | | | (H: Heavy Isotope, |
| | | Expected primary | S: Scissile Bond, C: |
| Peptide ID | Peptide | charged fragment | Control) |
| LAT3838 | AGSLDPAGSLR | PAGSLR⁺ | C |
| LAT3839 | A*G*S*LDPAGSLR | PAGSLR⁺ | H |
| LAT3840 | A*G*SLDPAGS*LR | PAGS*LR⁺ | H |
| LAT3841 | A*GSLDPAG*S*LR | PAG*S*LR⁺ | H |
| LAT3842 | AGSLDPA*G*S*LR | PA*G*S*LR⁺ | H |
| LAT3843 | AGSLADPGSLR | PGSLR⁺ | S |
| LAT3844 | AGSDPLAGSLR | PLAGSLR⁺ | S |
| LAT3845 | ADPGSLAGSLR | PGSLAGSLR⁺ | S |
| LAT3846 | AGSLAGSLDPR | PR⁺ | S |

The peptides and primary charge fragments are SEQ ID NO:2 and amino acids 6-11 of SEQ ID NO:2 for LAT3838, LAT3839, LAT3840, LAT3841, and LAT3842; SEQ ID NO:4 and amino acids 7-11 of SEQ ID NO:4 for LAT3843; SEQ ID NO:7 and amino acids 5-11 of SEQ ID NO:7 for LAT3844; SEQ ID NO:8 and amino acids 3-11 of SEQ ID NO:8 for LAT3845; and SEQ ID NO:27 and amino acids 10-11 of SEQ ID NO:27 for LAT3846. In Table 4, an asterisk indicates cold heavy isotope amino acid in the case of gly and ala, phosphoserine in the case of serine. For LAT3839, the fragment is distinguishable from control, but the parent ion is distinguishable. LAT3845 and LAT3846 exhibit an end of peptide effect.

ii. Second Peptides

Based upon the results with the first peptides, six additional peptides (to serve as reporter signals) were synthesized to demonstrate further points, including reversing the sequence, adding a terminal cysteine (to facilitate sulfur bridge covalent coupling) and addition of tyrosine (to allow for UV quantitation). These reporter signals are shown in Table 5. The set KER4086-KER4090 contain tryptophan to allow for quantitation using UV absorbance. The peptides and primary charged fragments are SEQ ID NO:28 and amino acids 8-14 of SEQ ID NO:28 for KER4086; SEQ ID NO:29 and amino acids 9-14 of SEQ ID NO:29 for KER4076; SEQ ID NO:30 and amino acids 10-14 of SEQ ID NO:30 for KER4088; SEQ ID NO:31 and amino acids 11-14 of SEQ ID NO:31 for KER4089; SEQ ID NO:32 and amino acids 12-14 of SEQ ID NO:32 for KER4090; and SEQ ID NO:33 and amino acids 1-6 of SEQ ID NO:33 for KER4120.

TABLE 5

| | | Expected primary | |
|---|---|---|---|
| Peptide ID | Peptide | charged fragment | Addresses |
| KER4086 | CGWAGSDPLAGSLR | PLAGSLR⁺ | UV quantitation |
| KER4087 | CGWAGSLDPAGSLR | PAGSLR⁺ | UV quantitation |
| KER4088 | CGWAGSLADPGSLR | PGSLR⁺ | UV quantitation |
| KER4089 | CGWAGSLAGDPSLR | PSLR⁺ | UV quantitation |
| KER4O9O | CGWAGSLAGSDPLR | PLR⁺ | UV quantitation |
| KER4120 | RLSGADPLSGAWGC | RLSGAD⁺ | Sequence direction |

iii. Instrumentation

The preferred mode for the disclosed method makes use of mass spectrometry. Mass spectrometers consist of three major categories of modules: source, filter/ion guide/analyzer, and detector. Commonly used sources for biological applications include Matrix Assisted Laser Desorption Ionization, MALDI, and Electrospray Ionization, ESI. Detectors on current instrumentation are generally Microchannel Plate, MCP, with a number of other detectors available. Between the source and the detector are any number of filters, ion guides, collision cells, laser excitation regions, mass analyzers, etc.

The class of instrument used in this example is called tandem mass spectrometer. The specific instrument used for these experiments is shown schematically in FIG. 1. A preferred spectrometer would have a MALDI source rather than the ESI source (MALDI tends to product singly charged ions, ESI tens to produce multiply charged ions). The ESI source of the spectrometer used here served to provide a more stringent demonstration of the disclosed method.

iv. Results

1. DP Sequence, One Component

The first analysis was conducted using a single peptide (LAT3838) to demonstrate scissile bond cleavage in this instrument. To demonstrate scissile bond cleavage, an approximate 1 mg/ml solution of a single peptide (LAT3838—dissolved in 50% acetonitrile/50% water/0.2% formic acid) was loaded into the mass spectrometer.

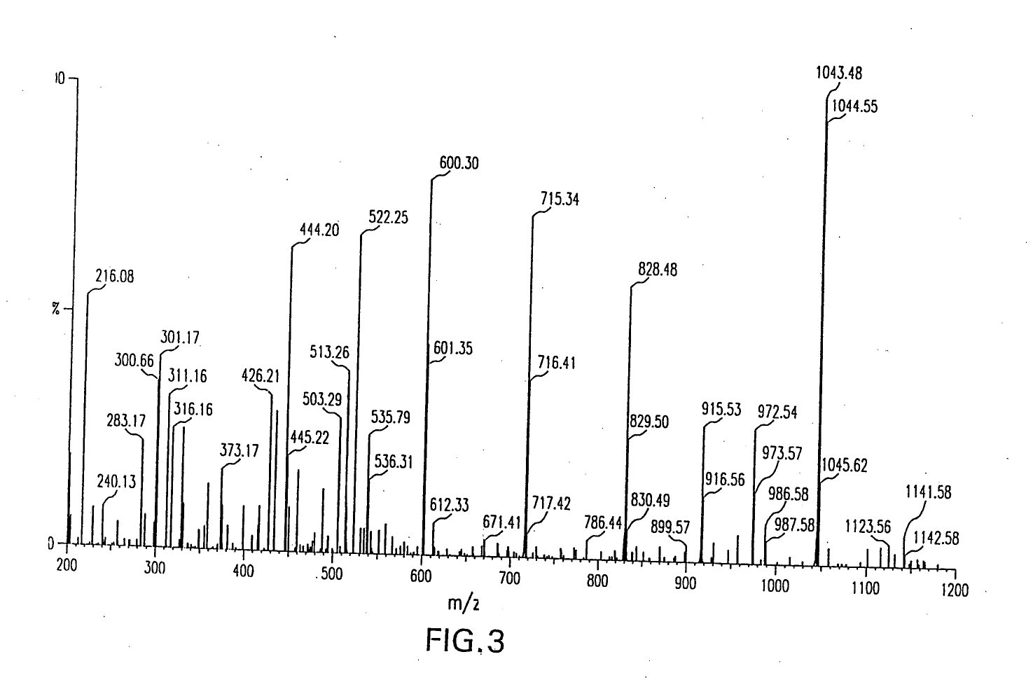

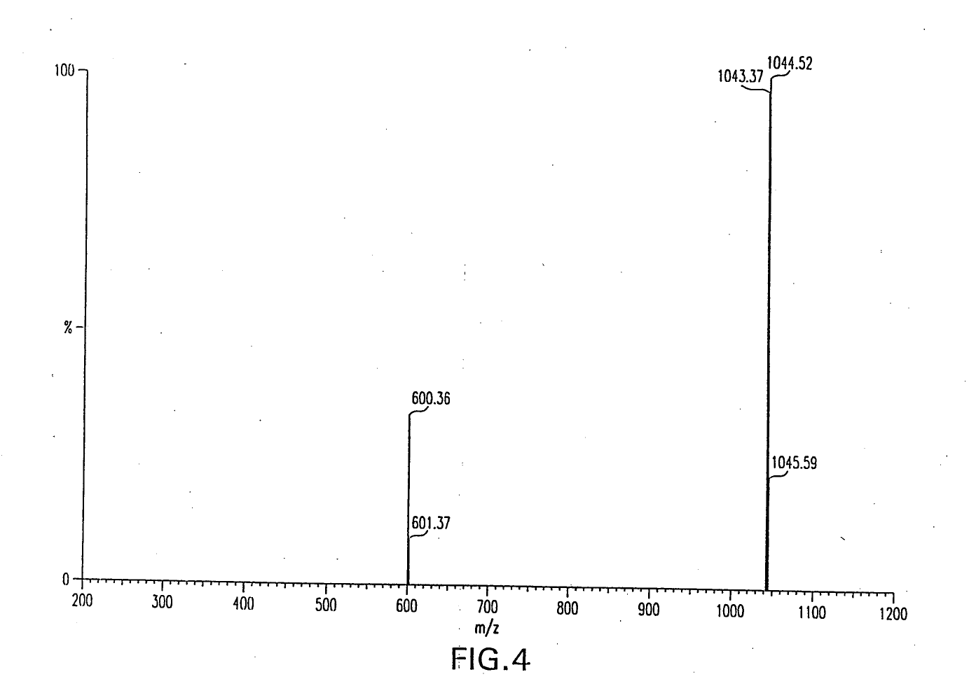

The sequence is AGSLDPAGSLR (SEQ ID NO:2) and was expected to fragment to a single daughter PAGSLR⁺ (amino acids 6-11 of SEQ ID NO:2). The complex ESI-TOF spectrum of AGSLDPAGSLR (SEQ ID NO:2) is shown in FIG. 3. This spectrum was generated when all peptides (the complete peptide and any fragments) from the ESI source are passed through the first resolving quadrupole, the collision cell, and into the TOF. The daughter ESI-MS/MS spectrum is shown in FIG. 4. To produce this spectrum, the resolving quadrupole operated as a mass filter to select for the parent peptide, allowing it to enter the collision cell while other peptides were not able to pass into the collision cell. Once in the collision cell, fragmentation occurred at the scissile DP bond as expected.

2. DP Sequence, Multiplexed

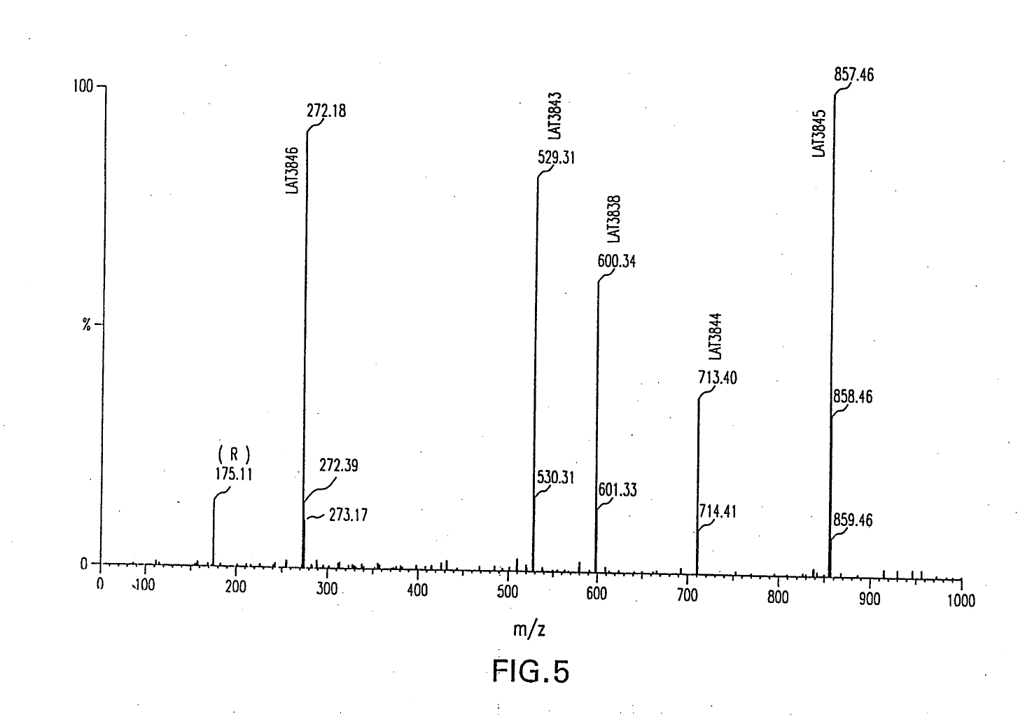

The set of peptides LAT3838 and LAT3843-3846 comprise an isobaric set of reporter signals. That is, all of the reporter signals in this set have the same mass-to-charge ratio. This set was mixed together in approximately equal concentration. 7 mg of each peptide was dissolved in 0.7 ml of 50% acetonitrile/50% water/0.2% formic acid. Dilutions of the stock samples were prepared and a final solution containing 0.1 μg/ml of each peptide was loaded into the mass spectrometer and subjected to ESI-MS/MS analysis. The parent spectrum was comparable to that shown in FIG. 3, the daughter ion spectrum, shown in FIG. 5, exhibits all five reporter signals at approximately equal amounts.

3. Heavy Isotopes

a. Phosphate Loss from Phosphorylated Serine

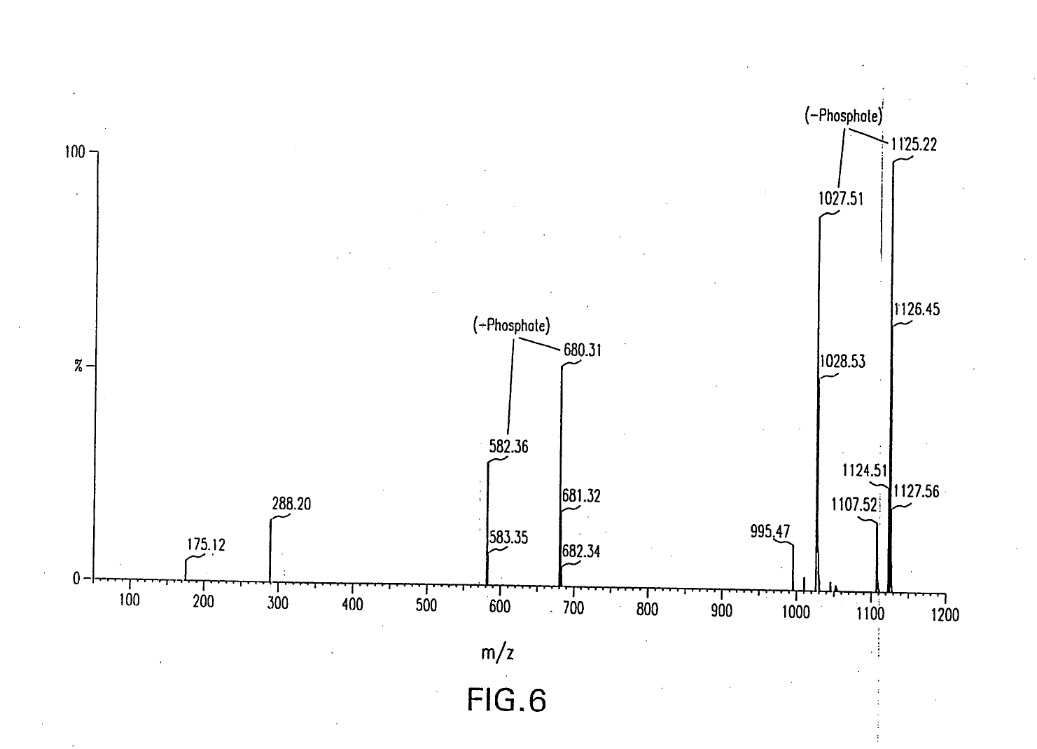

Spectra clearly show that phosphate loss is common in this system. This provides increased sampling, with two data points generated per reporter signal. A typical example of loss of phosphate from the phosphorylated serine is shown in FIG. 6.

b. Stable Isotope Amino Acids

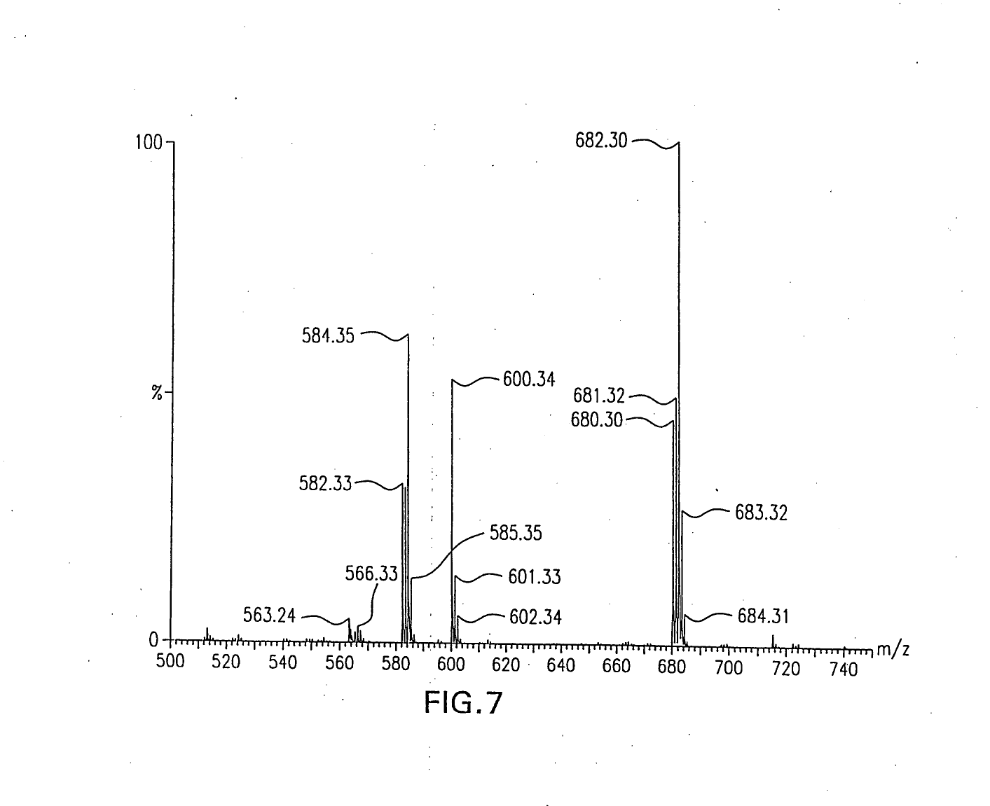

The stable isotopes incorporated into the reporter signals differed from the nature forms of the amino acids by 1 Dalton. As a consequence, naturally occurring heavy isotope peaks and the peak from the engineered reporter signals are at the same (degenerate) mass-to-charge. A range of mass-to-charge ratios is preferred for the disclosed method. The resulting traces were analyzed in a straightforward manner (see Table 6). It is clear from Table 6 and FIG. 7 that the “complicated peaks” correspond to three species and there are five measurements—the simultaneous equations can be solved for each fragment. Additionally, the peaks near m/z=582 and the peaks near m/z=680 correspond to the fragment less phosphate and fragment respectively. This information is redundant (assuming the loss of phosphate is a random chance event) and may be used to increase the quantitation confidence.

TABLE 6

| Observed m/z | Species responsible for signal |
|---|---|
| 600.34 | LAT3839_{M} |
| 601.33 | LAT3839_{M+1} |
| 602.34 | LAT3839_{M+2} |
| 680.30 | LAT3840_{M} |
| 681.32 | LAT3840_{M+1} + LAT3841_{M} |
| 682.30 | LAT3840_{M+2} + LAT3841_{M+1} + LAT3842_{M} |
| 683.31 | LAT3841_{M+2} + LAT3842_{M+1} |
| 684.31 | LAT3842_{M+2} |
| 582.33 | LAT3840_{M} − H₃PO₄ |
| 583.33 | LAT3840_{M+1} + LAT3841_{M} − H₃PO₄ |
| 584.35 | LAT3840_{M+2} + LAT3841_{M+1} + LAT3842_{M} − H₃PO₄ |
| 585.35 | LAT3841_{M+2} + LAT3842_{M+1} − H₃PO₄ |
| 568.35 | LAT3842_{M+2} − H₃PO₄ |

In Table 6, the observed m/z corresponds to that in FIG. 7. Nomenclature (not industry standard): PeptideID_{M} is fragment which contains no naturally occurring heavy isotope, PeptideID_{M+2} is the peak due to naturally occurring single mass occurrences of one unit heavy isotope of the fragment, PeptideID_{M+2} is the peak due to naturally occurring double mass unit heavy isotopes plus naturally occurring instances of two single mass heavy isotopes. Effect only up to M+2 are considered here but the method is extensible.

4. Second Peptides

The second set of peptides (reporter signals) is shown in Table 5 and specific aspects are addressed in these following sections. The data indicate the tryptophan, the cysteine, and the additional glycine in the second set of peptides behave well in the disclosed method.

a. Effect of Sequence

Sulfur bridge covalent bonding of reporter signals to specific binding molecules can be used. To facilitate this chemistry it is advantageous to have a cysteine as a terminal residue. Additionally, because peptide synthesis is from N to C terminus, if the cysteine is last on it can act as a purification element when the peptide is covalently attached to a specific binding molecule (compare to amine linker last on during oligonucleotide synthesis acts to purify the immobilized oligonucleotide).

To demonstrate the effect a C-terminal cysteine, peptide KER4120 (RLSGADPLSGAWGC; SEQ ID NO:33)) was synthesized which is precisely peptide KER4087 (CGWAGSLDPAGSLR; SEQ ID NO:29) in reverse sequence. The fragmentation pattern of KER4120 was similar to that of KER4087. This is consistent with a conclusion that synthesis of the peptide with the arginine on the N-terminus results in a peptide with a similar fragmentation pattern.

5. Complex Mixture

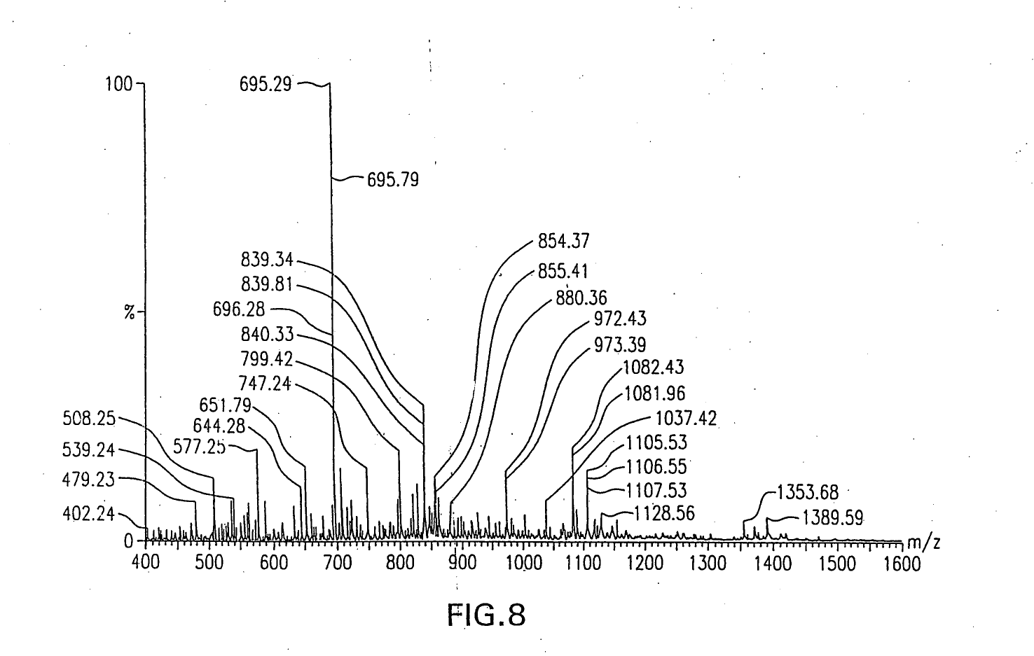

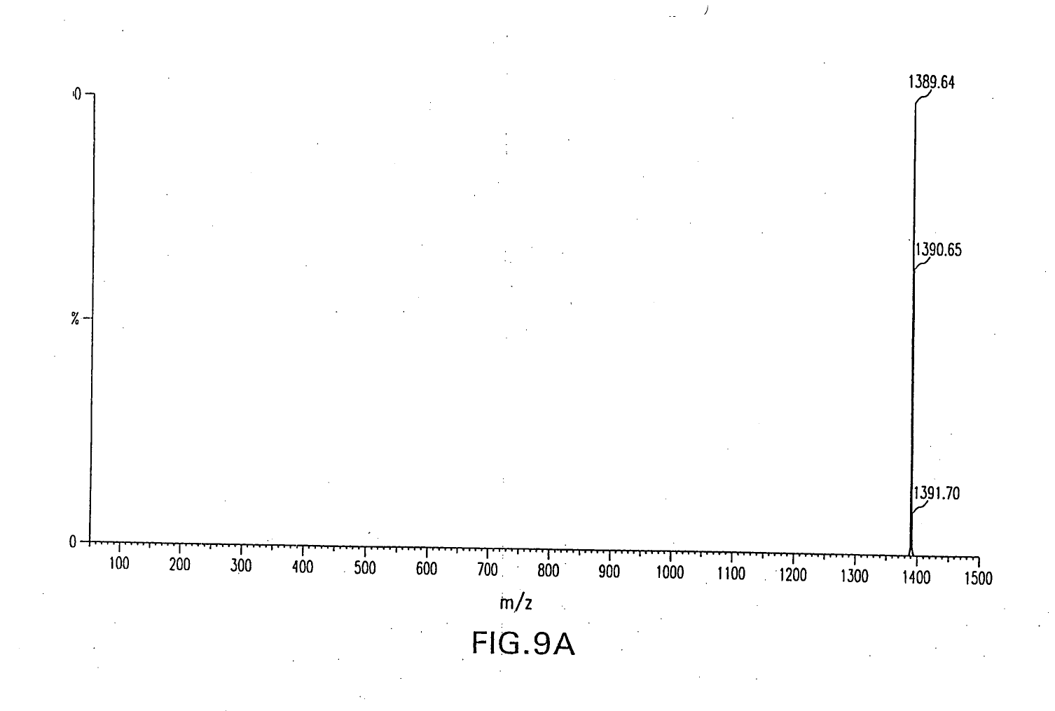

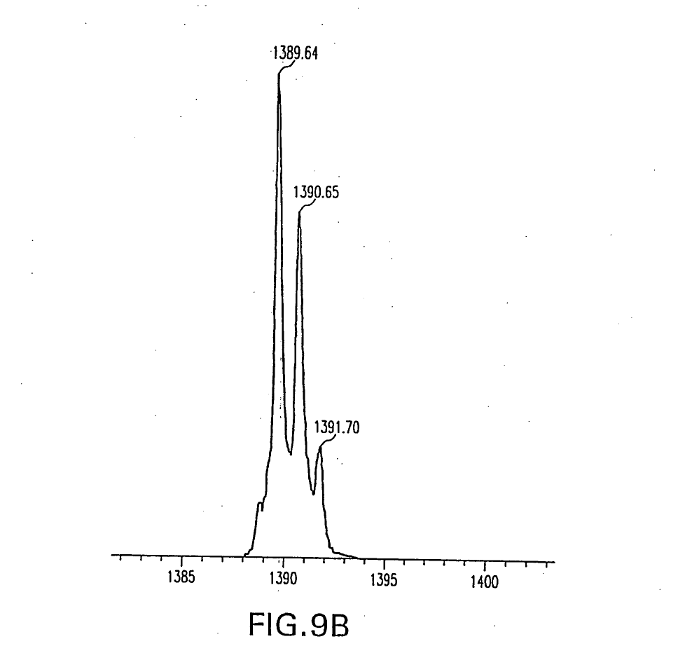

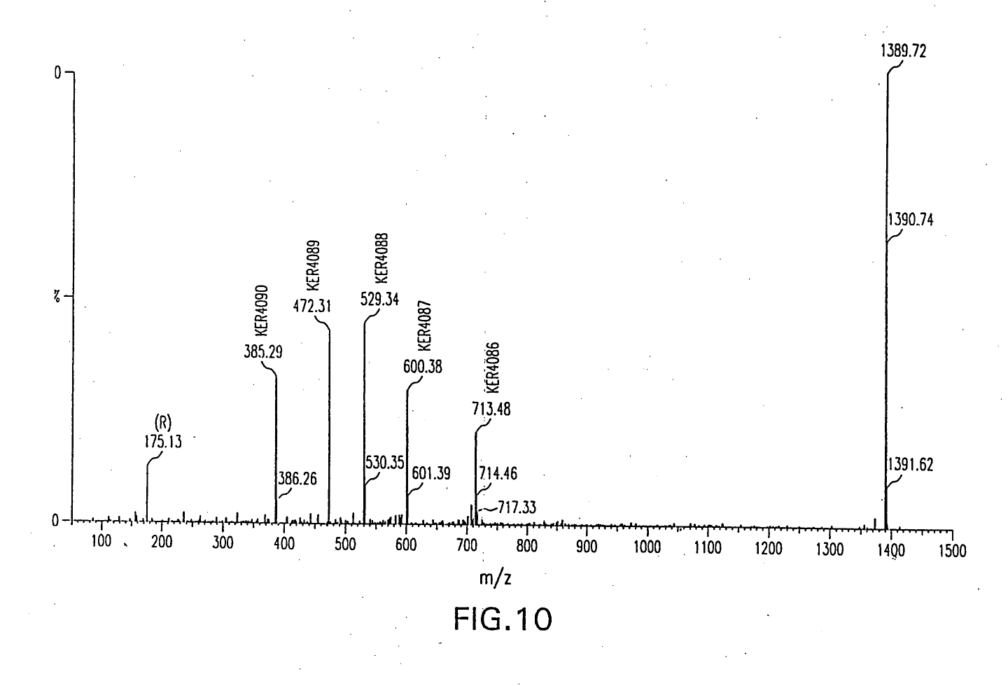

Clear demonstration of the power of the disclosed method is seen in an example measurement in a complex mixture. To generate a reasonably complex mixture Bovine Serum Albumin (BSA) (66 kDa) and creatine phosphokinase (84 kDa) were digested using trypsin. Five peptides (KER4086 to KER4090) were added to the digestion mixture (to a final concentration of 0.1 μg/ml of each peptide). The resulting mass spectrum for this complex mixture is shown in FIG. 8. As can be seen, the mixture is quite complex, showing peaks at a wide variety of mass-to-chare ratios. The spectrum following selection of the common mass-to-charge ratio is shown in FIG. 9. As can be seen, the filtering (selection) step produces a dramatic (essentially complete) reduction in complexity. The spectrum following fragmentation of the selected mass-to-charge fraction is shown in FIG. 10. As can be seen, clear peaks, all at nearly the same level (as expected based on equal amounts of starting material for each reporter signal), appear for each of the five expected reporter signal fragments (each having a distinctive mass-to-charge ratio. FIGS. 8 through 10 give a powerful representation of the progression from a complex mixture to the identification and determination of relative concentrations of specific labels.

### a. Example 2

### Use of Antibodies to Reporter Signals for Immuno-Precipitation of Reporter Signals and for Separation of Reporter Signals from Other Materials

This example describes an example of the use of specific binding molecules that bind to reporter signals. This example describes production of affinity capture beads having antibodies to reporter signals. Such beads can be used, for example, for immuno-precipitation of peptides labeled with signal peptides (in small) and for purification of such peptides (in large samples). This example also describes use of antibodies to reporter signals used in a Western blot to visualize proteins labeled with reporter signals.

i. Reporter Signals

The reporter signals used in this example were two forms of polyglycine reporter signals. In one set (referred to as the “light” labels; shown in Table 8), there are five reporter signals having 12 glycine residues with one aspartic acid-proline (DP) residue pair at a different position within the reporter signal. Fragmentation at the DP pair results in fragments of different sizes for each reporter signal. In another set (referred to as the “heavy” labels; shown in Table 7), there are seven reporter signals having the sequence GGGGGGDPGGGGGG (SEQ ID NO:39) where six of the glycine residues are composed of standard isotopes and the other six glycine residues are composed of heavy isotopes of (¹³C) carbon and nitrogen (¹⁵N).

ii. Antibody Production

Polyclonal antibodies to reporter signals containing 12 glycine residues and one aspartic acid-proline residue pair were prepared. Rabbits and goats were immunized with the immunogen KLH-[S-GGGGGGDPGGGGGG]n using standard protocols for antibody production (KLH=keyhole limpet hemocyanin). This involved one injection 300 μg immunogen+Complete Freund's Adjuvant (CFA) (week 0); three injections 200 μg immunogne+Incomplete Freund's Adjuvant (IFA) (weeks 3, 6, 9); and booster injections 200 μg immunogen+IFA (monthly). For antisera production, serum was obtained one week after booster shot. Antibodies were identified by ELISA using BSA-[S-GGGGGGDPGGGGGG]ₙ as the ELISA antigen. Labeled BAS was used as a control.

iii. Preparation of affinity capture beads

The total IgG fraction was obtained from 20 ml of Polyclonal anti-reporter signal rabbit serum (animal ID: 1225) by standard Protein A affinity purification. It is estimated that about 1% of these IgG antibodies are specific against the reporter signals. Total IgG was coupled to sepharose beads by cyanogen bromide (CNBr) mediated conjugation as follows. 46 mg total IgG were dialyzed into buffer containing 0.2 M NaHCO₃, 0.5 M NaCl (pH 8.5) and conjugated to 5 ml preactivated resin (CNBr-activated Sepharose 4 Fast Flow media from Pharmacia) for 4 hours at room temperature. Coupling efficiency was 71%. The coupled resin was washed in the same buffer, then rinsed with phosphate buffered saline (PBS). The final resin prep was stored at 4° C. in PBS containing 0.01% sodium azide.

iv. Capture of Reporter Signals Using Affinity Capture Beads and Analysis of Reporter Signals

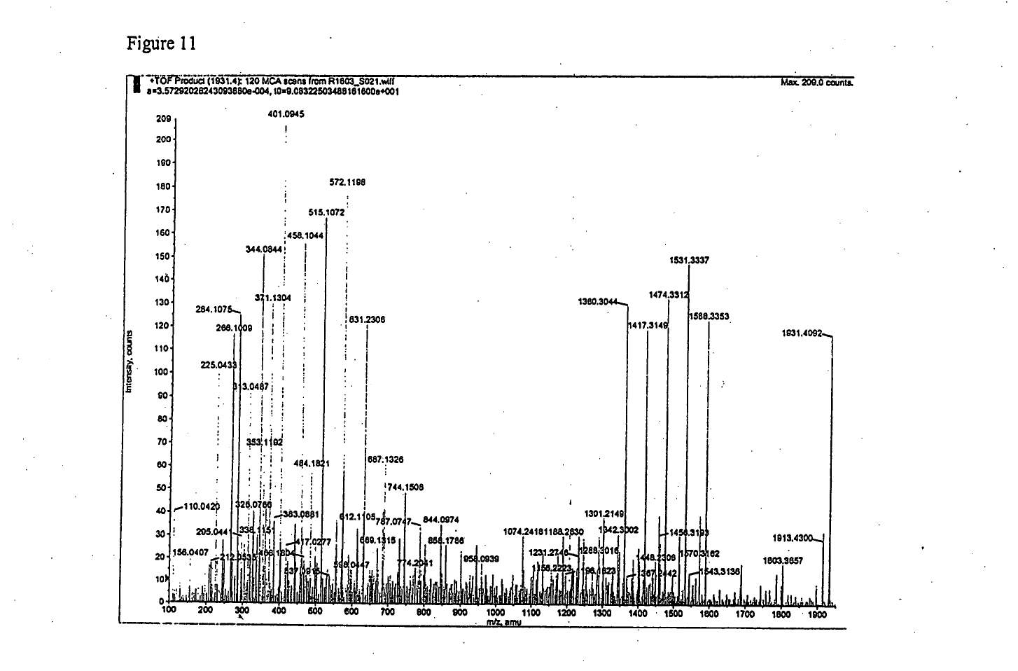

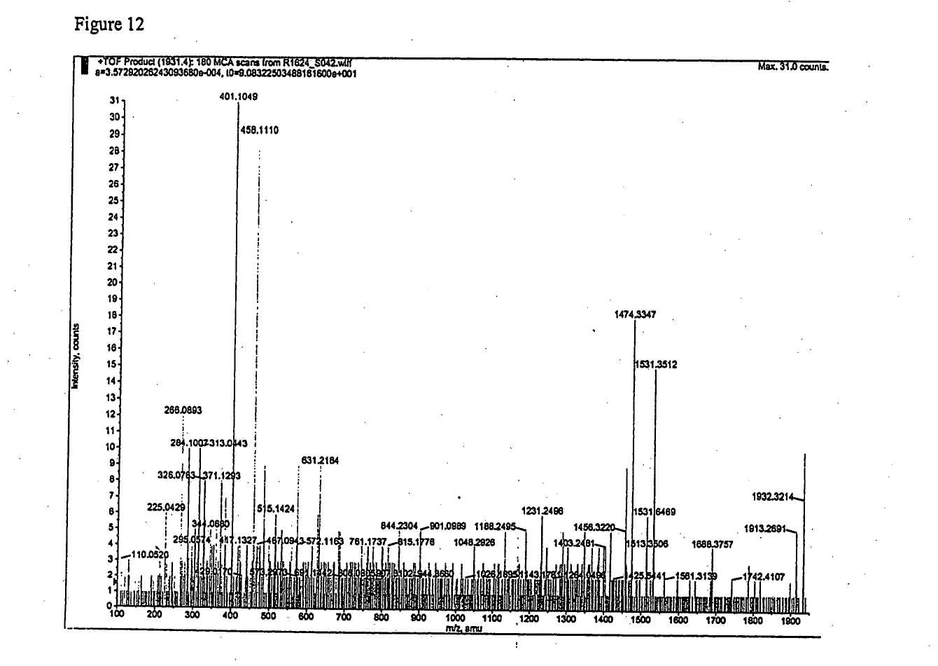

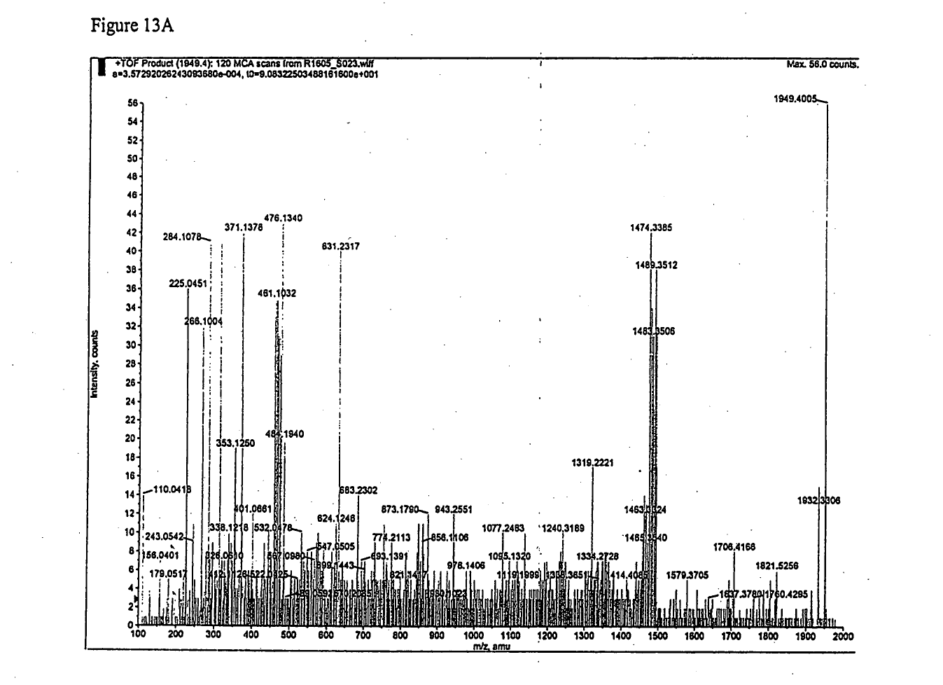

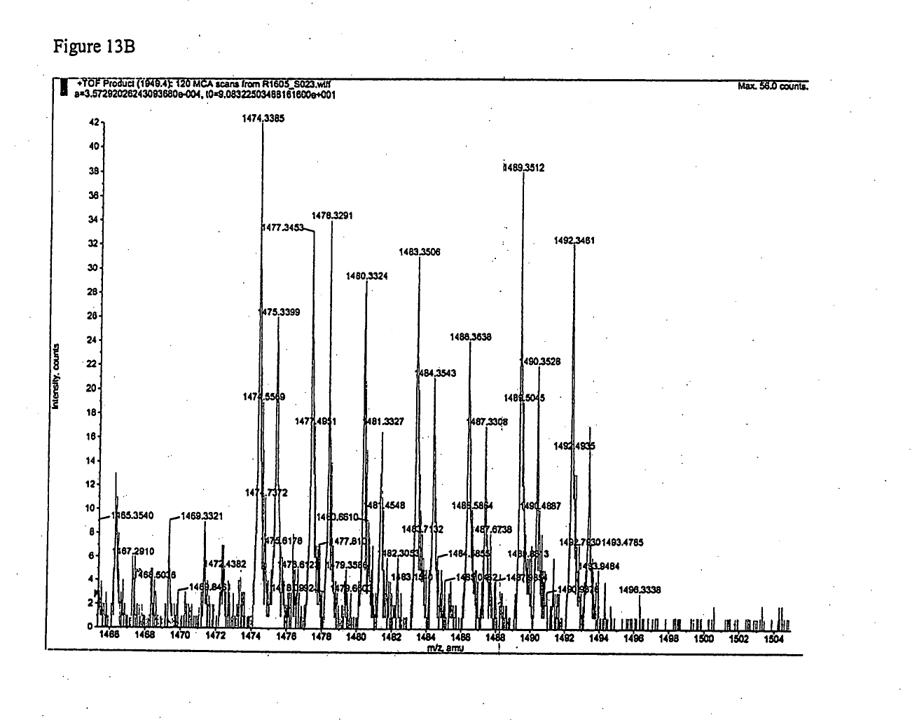

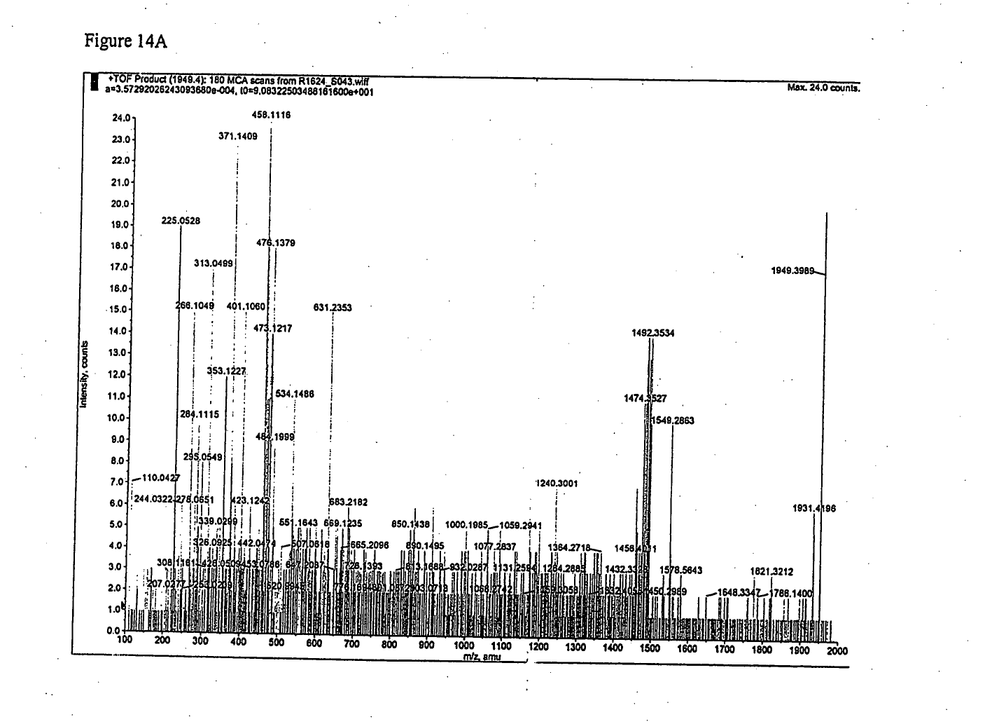

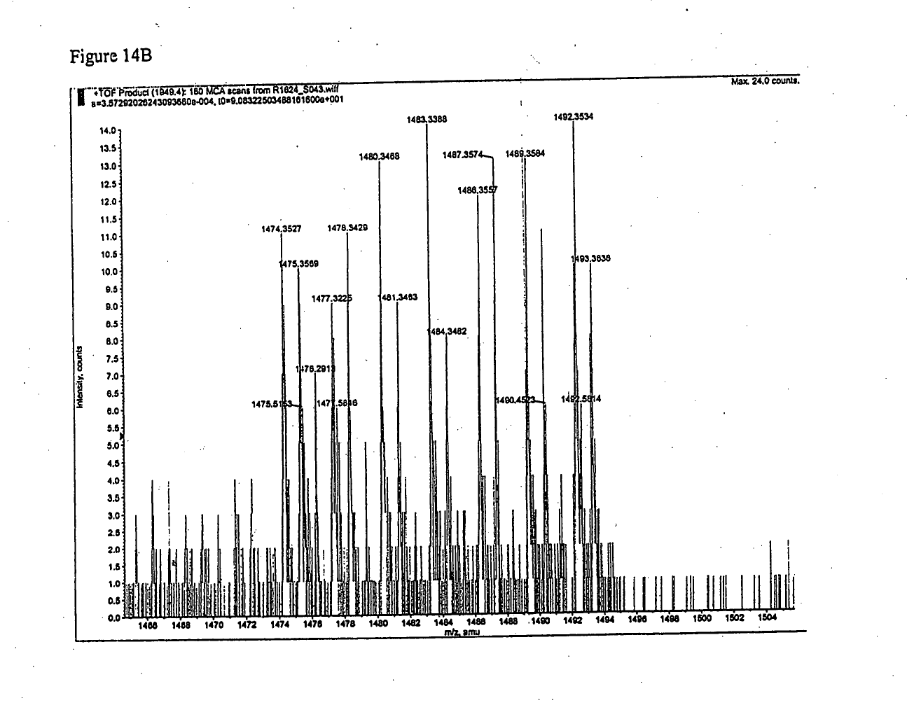

BSA was labeled with the reporter signals from the two sets and fragmented with trypsin. This is a model of the use of reporter signals to label proteins. Affinity capture beads with polyclonal antibodies to the immunogen reporter signal were incubated with the labeled BSA fragments in binding buffer (100 mM Tris pH 7, 0.14 M NaCl) for two hours with agitation at 37° C. The beads were washed three times in wash buffer (10 mM Tris pH 7, 0.14 M NaCl). The bound protein fragments were eluted for 15 minutes at 24.5° C. using elution buffer (0.1 M Glycine pH 2.3). Reserved samples of labeled BSA fragments prior to binding to affinity capture beads and eluted labeled BSA fragments were analyzed by liquid chromatography time-of-flight two stage mass spectrometry (LC/TOF/MS/MS). Fractions were collected from C18 column and spotted on MALDI source. TOF analysis was used to detect fractions with labeled peptides. MS/MS analysis of selected fractions was used to detect reporter signals. The results are shown in FIGS. 11-14. All of the reporter signals were detected in the reserved samples prior to binding to the affinity capture beads. In the eluted samples, all of the “heavy” reporter signals (Table 7) were detected. However, only two of the “light” reporter signals (Table 8; SEQ ID NOs:36 and 37) were represented. This likely represents loss of specificity of the antibody due to differences between the DP location in the other reporter signals and the peptide used to raise the antibodies.

v. Western Blotting Using Antibodies to Reporter Signals

The antibodies described above were use for the detection of proteins labeled with reporter signals after separation by SDS-polyacrylamide gel electrophoresis (PAGE) and blotting onto a nitrocellulose or other suitable membrane substrate.

The protein samples are fractionated by one dimensional (1D) or two dimensional (2D) SDS-PAGE and the gel is blotted onto a suitable membrane by standard procedures. Labeled proteins are detected by chemiluminescence as bands (1D gels) or spots (2D gels). Detection procedure: Block the membrane using a blocking buffer as recommended by the manufacturer. Dilute 1 ul of Polyclonal anti-reporter signal rabbit serum (animal ID:1225) into 10 ml of blocking buffer (1:10,000 dilution) to prepare the 1^{st} antibody solution. Soak the membrane in this solution for 1 hour at room temperature. Pour antibody off and rinse 4 times with water. Rinse the membrane three times for five minutes in blocking buffer. Dilute 1 μl of Goat anti-rabbit HRP-conjugated antibody (0.4 mg/ml) into 100 ml of blocking buffer (1:100,000 dilution) to prepare the 2^{nd} antibody solution. Soak the membrane in this solution and wash as described for the 1^{st} antibody solution. Do a final rinse in water and develop the blot for chemiluminescence using the Rabbit IgG Detection kit from PIERCE. Following such detection, reporter signals can be collected (by elution or extraction, for example) and processed by, for example, separation based on a common property, alteration of the reporter signals, and detection of the altered forms of the reporter signals.

It is understood that the disclosed invention is not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a host cell” includes a plurality of such host cells,

Disclosed are the components to be used to prepare the disclosed compositions as well as the compositions themselves and to be used within the methods disclosed herein. These and other materials are disclosed herein, and it is understood that when combinations, subsets, interactions, groups, etc. of these materials are disclosed that while specific reference of each various individual and collective permutation of these compounds may not be explicitly disclosed, each is specifically contemplated and described herein. For example, if a particular type of reporter signal is disclosed and discussed in the context of some modes and embodiments of the disclosed method, specifically contemplated is each and every combination and permutation of the reporter signal and the various forms and embodiments of the disclosed method that are possible unless specifically indicated to the contrary. Thus, if a class of molecules A, B, and C are disclosed as well as a class of molecules D, E, and F and an example of a combination molecule, A-D is disclosed, then even if it each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Unless defined otherwise, all technical and scientific terms used herein have the same meanings as commonly understood by one of skill in the art to which the disclosed invention belongs. Although any methods and materials similar or equivalent to those described herein can be used in the practice or testing of the present invention, the preferred methods, devices, and materials are as described. Publications cited herein and the material for which they are cited are specifically incorporated by reference. Nothing herein is to be construed as an admission that the invention is not entitled to antedate such disclosure by virtue of prior invention.

Those skilled in the art will recognize, or be able to ascertain using no more than routine experimentation, many equivalents to the specific embodiments of the invention described herein. Such equivalents are intended to be encompassed by the following claims.

We claim:

1. A binding molecule specific for a plurality of reporter signals in a set of reporter signals, wherein the set of reporter signals comprises a plurality of reporter signals,
wherein the reporter signals have a common property, wherein the common property allows the reporter signals to be distinguished or separated from molecules lacking the common property,
wherein the reporter signals can be altered, wherein the altered forms of each reporter signal can be distinguished from every other altered form of reporter signal.

2. The binding molecule of claim 1, wherein the binding molecule is an antibody.

3. The binding molecule of claim 1 wherein the common property is mass-to-charge ratio, wherein the reporter signals are altered by altering their mass, wherein the altered forms of the reporter signals can be distinguished via differences in the mass-to-charge ratio of the altered forms of reporter signals.

4. The binding molecule of claim 3 wherein the mass of the reporter signals is altered by fragmentation.

5. The binding molecule of claim 1 wherein the common property is mass-to-charge ratio, wherein the reporter signals are altered by altering their charge, wherein the altered forms of the labeled proteins can be distinguished via differences in the mass-to-charge ratio of the altered forms of reporter signals.

6. The antibody of claim 1 wherein the set comprises two or more, three or more, four or more, five or more, six or more, seven or more, eight or more, nine or more, ten or more, twenty or more, thirty or more, forty or more, fifty or more, sixty or more, seventy or more, eighty or more, ninety or more, or one hundred or more different reporter signals.

7. The binding molecule of claim 6 wherein the wherein the reporter signals are associated with, or coupled to analytes.

8. The binding molecule of claim 7, wherein the analytes are proteins or peptides.

9. The binding molecule of claim 1 wherein the reporter signals are comprised of molecules selected from the group consisting of peptides, oligonucleotides, carbohydrates, polymers, oligopeptides, and peptide nucleic acids.

10. The binding molecule of claim 1, wherein the reporter signals are peptides.

11. The binding molecule of claim 10, wherein the reporter signal peptides in the set each has a characteristic selected from the group consisting of each reporter single peptide having the same amino acid sequence, each reporter signal peptide containing a different distribution of heavy isotopes, each reporter signal peptide containing a different distribution of substituent groups, each reporter signal peptide having a different amino acid sequence, each reporter signal peptide having a labile or scissile bond in a different location, and each reporter signal peptide having a labile or scissile bond in a same location

12. A set of binding molecules specific for a plurality of reporter signals in a set of reporter signals, wherein the set of reporter signals comprises a plurality of reporter signals,
wherein the reporter signals have a common property, wherein the common property allows the reporter signals to be distinguished or separated from molecules lacking the common property,
wherein the reporter signals can be altered, wherein the altered forms of each reporter signal can be distinguished from every other altered form of reporter signal, and wherein each reporter signal is associated with, or coupled to, a each binding molecule in the set is specific for a different reporter signal in the set.

13. The set of binding molecules of claim 12, wherein the binding molecules are antibodies.

14. The set of binding molecules of claim 12, wherein the wherein the reporter signals are associated with, or coupled to analytes.

15. A method of detecting a protein or peptide, the method comprising
(a) separating, detecting, sorting, or immobilizing a set of reporter signals by binding the a plurality of the reporter signals in the set to a specific binding molecule, and wherein each reporter signal in the set shares a common property;
(b) altering the reporter signals; and
(b) detecting and distinguishing the altered forms of the reporter signals from each other.

16. The method of claim 1, wherein the reporter signals are associated with, or coupled to analytes.

17. The method of claim 15, wherein the binding molecule is an antibody.

18. A kit comprising
(a) a set of reporter signals, wherein the reporter signals have a common property, wherein the common property allows the reporter signals to be distinguished or separated from molecules lacking the common property, wherein the reporter signals can be altered, wherein the altered forms of each reporter signal can be distinguished from every other altered form of reporter signal, wherein each different reporter molecule comprises a different decoding tag and a different reporter signal, and
(b) a binding molecule specific for a plurality of the reporter signals.
